(19) United States
(12) Patent Application Publication
(10) Pub. No.: US 2016/0084855 A1
(43) Pub. Date: Mar. 24, 2016

(54) Compositions and Methods for Modulation and Detection of Immune and Inflammatory Responses

(71) Applicant: UNIVERSITY OF CONNECTICUT, Farmington,, CT (US)

(72) Inventors: Robert B. Clark, Farmington, CT (US); Frank C. Nichols, West Hartford, CT (US)

(73) Assignee: University of Connecticut, Farmington, CT (US)

(21) Appl. No.: 14/784,717

(22) PCT Filed: Apr. 18, 2014
(86) PCT No.: PCT/US14/34645

§ 371 Date: Oct. 15, 2015

Related U.S. Application Data
(60) Provisional application No. 61/813,473, filed on Apr. 18, 2013.

(51) Int. Cl. G01N 33/92; C07K 16/12; G01N 30/72; A61K 38/05

(52) U.S. Cl. CPC G01N 33/92; A61K 38/05; C07K 16/1203; G01N 30/72; G01N 2333/195; G01N 2405/08; G01N 2560/00; G01N 2800/285

(57) ABSTRACT

Methods for detecting inflammatory or autoimmune conditions, comprising analyzing bacterial L-serine containing lipids in a sample; and, comparing results of the analysis of the bacterial L-serine containing lipids in the sample with information on occurrence of the bacterial L-serine containing lipids in a control sample, wherein a decreased occurrence of the bacterial L-serine containing lipids in the test sample over the occurrence of bacterial L-serine containing lipids in the control sample indicates the presence of an inflammatory or an autoimmune condition, are described herein. An example of the autoimmune condition is multiple sclerosis (MS). The use of bacterial L-serine containing lipids as biomarkers for detection of MS is described. Antibodies specific to L-serine containing lipids and their uses are also provided. Also provided are compositions comprising bacterial L-serine containing lipids for modulating immune responses or TLR pathways in humans, animals, and human or animal cells or tissues.

### CROSS-REFERENCE TO PRIORITY APPLICATION

This application claims priority to U.S. Provisional Application No. 61/813,473, filed Apr. 18, 2013, which is incorporated herein by reference in its entirety.

### STATEMENT REGARDING FEDERALLY FUNDED RESEARCH

This invention was made with government support under Grant No. 1 R01 DE021055-01A1 awarded by the National Institutes of Health/National Institute of Dental and Craniofacial Research. The government has certain rights in the invention.

### FIELD

This application relates to the general field of compositions and methods for modulation and detection of immune and inflammatory responses.

### BACKGROUND

A number of recent reports focused on the role of bacteria commonly inhabiting human bodies, or commensal bacteria, in the functioning of immune system and human disease. In particular, commensal bacterial were implicated in development and regulation of inflammatory and autoimmune diseases or conditions. See, for example, Wen et al., “Innate immunity and intestinal microbiota in the development of Type 1 diabetes” Nature 455:1109-1113 (2008); Yokote et al., “NKT cell-dependent amelioration of a mouse model of multiple sclerosis by altering gut flora” Am. J. Pathol. 173:1714-1723; Mazmanian et al., “A microbial symbiosis factor prevents intestinal inflammatory disease” Nature 453:620-625 (2008). However, this information was not translated into useful medical or diagnostic applications.

Inflammatory responses characterize a large group of normal and pathologic diseases and conditions in humans or animals. Inflammatory responses are a group of complex biological responses, which typically involve vascular changes and cellular infiltration, of animal cells and tissues to harmful stimuli, such as pathogens, damaged cells, or irritants Immune system involvement in some inflammatory responses, such as those seen in allergies and autoimmune disorders, is well known. Involvement of the immune system in some other inflammatory events, such as those observed in cancer, atherosclerosis, and ischemic heart disease, is less well established, although such a possibility is recognized. Inflammatory events involve a large variety of tissue, cellular and molecular events and mechanisms. A number of useful inflammation biomarkers are known, but there is a continuing need for both clinical and research biomarkers, and methods for assessing inflammatory states that would possess improved reproducibility, biological variability, analytic variability, sensitivity and specificity, as well as large-scale feasibility.

Autoimmune diseases and conditions are a large group of diseases and conditions, which includes dozens of important and debilitating human diseases and disorders in which the immune system attacks the host's own tissues and cells. It is thought that each autoimmune disease is most likely caused by a combination of different factors, and even classification of a disease or a condition as autoimmune is complicated. For example, according to one convention accepted in the medical field, for a disease to be regarded as an autoimmune disease, it needs to answer to the so-called Witebsky's postulates, first formulated by Ernst Witebsky and colleagues in 1957, which include direct evidence from transfer of pathogenic antibody or pathogenic T cells, indirect evidence based on reproduction of the autoimmune disease in experimental animals, and circumstantial evidence from clinical clues. Examples of diseases typically regarded as autoimmune are rheumatoid arthritis, systemic lupus erythematosus (SLE), diabetes (type 1), and multiple sclerosis (MS). Autoimmune diseases often have variable symptoms and courses and do not always restrict themselves to one part of the body. For example, SLE can affect the skin, joints, kidneys, heart, nerves, blood vessels, and more. In some patients, rheumatoid arthritis can affect the heart, blood vessels and lungs, in addition to the joint problems it typically causes. Autoimmunity may also play a role in the development of atherosclerosis. While it is currently understood that the immune system in most individuals has the potential to attack self-tissues, the factors that lead to autoimmune diseases in only a subset of individuals remain unknown. The difficulties in classifying and diagnosing autoimmune diseases and conditions contribute to a continuing need for biomarkers and methods for diagnosing and assessing autoimmune diseases and conditions, both in the clinical and research contexts.

For some relatively common autoimmune diseases, no biomarkers are currently known and no straightforward diagnostic methods exist. One such disease is multiple sclerosis (MS), which is generally considered to be an autoimmune disease. MS is currently characterized as a human disease in which the immune system targets and attacks the myelin sheath that surrounds and protects the nerve fibers of the central nervous system (CNS). The resulting damage to the myelin and the nerve fiber greatly disrupts the normal flow of electrical impulses to and from the brain, resulting in the various symptoms of MS.

The diagnosis of MS is very difficult and there is no single test that confirms MS in a patient. Typically, physicians require a detailed medical history including the symptoms experienced by the patient; a careful physical exam, including tests of coordination, strength and reflexes; and a number of laboratory tests on samples of blood or cerebrospinal fluid (CSF) to try to rule out other possible causes for the symptoms experienced by the patient. A preferred test is magnetic resonance imaging (MRI) of the brain, which can detect plaques, lesions or scarring which might be caused by MS. However, MRIs have problems with both sensitivity and specificity. A test of Visual Evoked Potentials (VEP), which studies the speed of electrical signals in parts of the brain, may also be used. However, a course and progression of MS is highly variable between patients and is very hard to predict for a given individual. Many patients experience episodes of serious disease symptoms separated by months or more of at least partial remission. At present, there are no known biological markers or methods employing such biomarkers that predict disease activity for MS.

### SUMMARY

The terms “invention,” “the invention,” “this invention” and “the present invention” used in this patent are intended to refer broadly to all of the subject matter of this patent and the patent claims below. Statements containing these terms should be understood not to limit the subject matter described herein or to limit the meaning or scope of the patent claims below. Embodiments of the invention covered by this patent are defined by the claims below, not this summary. This summary is a high-level overview of various aspects of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification, any or all drawings and each claim.

Disclosed herein are methods for detecting a disease or a condition, or detection methods, which involve, in any combination, detecting, testing or analyzing bacterial lipids present in a cell or a tissue sample obtained from a human or an animal. In some embodiments of the present invention, bacterial lipids under analysis are bacterial lipids that are not synthesized by the human or the animal, which are referred to as “bacteria-originated lipids.” In one variation, bacteria-originated lipids are synthesized by commensal bacteria living in various parts of human or animal organisms.

Detection of a disease or a condition according to various embodiments of the detection methods disclosed herein can employ appropriate analytical methods, techniques or procedures. In some embodiments of the detection methods, mass-spectrometry is employed in the analysis of bacterial lipids. In some other embodiments, immunochemical techniques are employed.

According to some of the embodiments of the present invention, bacteria-originated lipids are used as biological markers, or biomarkers, for detection of diseases and conditions. For example, patterns of bacteria-originated lipids detected by an analytical method in a sample obtained from a human or animal correlate with a presence, absence, state or degree of a disease or condition. Such patterns therefore can be used in the methods for detecting diseases and conditions.

Also disclosed herein are antibodies against bacteria-originated lipids and uses of such antibodies. For example, antibodies against bacteria-originated lipids are used in methods of detecting a disease or a condition, methods of modulating immune or inflammatory responses in the humans or the animals or in the human or animal cells, or in therapeutic and diagnostic methods related to diseases and conditions. Antibodies against the bacteria-originated lipids are also used in medicaments, pharmaceutical compositions, research, analytical and diagnostic compositions, tools, kits and reagents related to treatment and detection of various diseases and conditions or modulation of immune or inflammatory responses in human or animal cells and organisms, as well and in the research activities related to such treatment and detection.

Some embodiments of the methods described herein are methods for detection of inflammatory or autoimmune diseases, conditions or states. Examples of such inflammatory diseases, conditions or states are provided elsewhere in this document. Some other embodiments of the methods disclosed herein are useful for detection of multiple sclerosis, or MS. One such embodiment is a method for detecting MS biomarkers. In one example, the method for detecting MS biomarkers employs an analysis of a blood sample. The method is useful for diagnosing, assessing, monitoring, and following the progression of MS. It is also useful in MS prognosis and prediction. For example, it is useful for predicting exacerbation of symptoms in patients with MS. The method is also useful for monitoring and evaluating the efficacy of clinical treatments for MS. Generally, the methods, biomarkers, molecules, such as antibodies, and other elements disclosed herein provide the first blood test for detection of MS.

As disclosed herein, patients with MS have a pattern of bacteria-originated lipids in samples of some of their tissues, such as blood and brain tissues, or in bacterial samples obtained from the patients' bodies, the pattern being detectably different from a pattern of bacteria-originated lipids in the corresponding samples obtained from MS-free control subjects. By way of example, some of the bacteria-originated lipids originate from commensal bacteria, such as Porphyromonas gingivalis that is often present in the oral cavity. Among the novel lipids of such bacteria are phosphorylated dihydroceramides (PDHCs). Two major classes of PDHCs are phosphoethanolamine dihydroceramides (PE DHCs) and phosphoglycerol dihydroceramides (PG DHCs). These two lipid classes have different biological activities related to specific structural components present in each class. Further lipids of such bacteria include L-serine containing lipids. Examples of L-serine containing lipids include Lipid 654 and Lipid 430.

In one exemplary embodiment of the present invention, the bacterial lipids present in human serum or other fluids are characterized and quantitated using MRM (multiple reaction monitoring) mass spectrometry. MRM-mass spectrometry is the approach used in this embodiment because it provides the advantages of most specific identification and quantification of the lipid families. The methods disclosed herein include analysis of samples of obtainable bodily fluids, specifically serum and cerebrospinal fluid, but also including synovial fluid, tears, and lymphatic fluid. Tissue samples may also be assessed by the disclosed methods. In an exemplary embodiment, monoclonal antibodies are generated to specific PDHC lipids and L-serine containing lipids, and such monoclonal antibodies are used in an ELISA to detect the presence, quantity and pattern of serum bacterial lipids in an individual.

Also disclosed herein are compositions comprising bacteria-originated lipids useful for modulation of immune or inflammatory responses, activation of toll-like receptors (TLRs) or modulation of their activity, as well as modulation of toll-like receptor signaling pathways (“TLR pathways”) and binding to TLRs in humans, animals, and human or animal cells tissues, along with corresponding methods and uses of such compositions. According to some embodiments of the present invention, bacteria-originated lipids are used in medicaments, pharmaceutical compositions, research, analytical and diagnostic compositions, tools, kits and reagents related to treatment and detection of various diseases and conditions, modulation of immune or inflammatory responses, modulation of TLR pathways, binding to TLRs, and in the therapeutic, diagnostic and research activities related to immune and inflammatory pathways, TLRs and TLR pathways, and any related diseases, conditions or states.

### BRIEF DESCRIPTION OF THE DRAWINGS

Illustrative embodiments of the present invention are described in detail below with reference to the following drawing figures:

FIG. 1 is a schematic representation of the chemical structures of bacterial PDHCs.

FIG. 2 is a bar graph schematically representing the results of the analysis of bacteria-originated PDHCs recovered from intestinal and oral bacterial samples. The ion abundances of high and low mass PDHC lipid classes were summed and the recovery of each lipid class is depicted as the percent of the total ion abundance of the quantified PDHCs. Standard deviation bars are shown for lipid extracts from Bacteroides vulgatus (n=13), Prevotella capri (n=2), and Porphyromonas gingivalis (n=6).

FIG. 3 is a bar graph schematic representation of the analysis of bacteria-originated PDHCs recovered from subgingival plaque samples (n=2), samples of healthy/mildly inflamed gingival tissue (GT H+G, n=7), periodontitis gingival tissue samples (GT Perio, n=6), blood plasma samples from periodontally healthy subjects (Blood Cont, n=8), blood plasma from patients with generalized severe periodontitis (Blood Perio, n=7), carotid atheroma (Atheroma, n=11) and postmortem brain samples from non-MS subjects (Brain Control, n=14). The ion abundances of high and low mass PDHC lipid classes were summed and the recovery of each lipid class is depicted as the percent of the total ion abundance of the quantified PDHC lipids. Standard deviation bars are shown.

FIG. 4 is a bar graph schematically representing the results of the analysis of bacteria-originated PCHCs in paired patent artery and atheroma samples. For each carotid atheroma, the patent artery segment of the proximal common carotid artery was excised from the gross atheroma located within the carotid sinus. A defined amount (approximately 3 μg of total lipids in 5 μl of HPLC solvent) of each lipid extract was analyzed by MRM MS/MS and the recovery of each lipid class is depicted as the percent of the total ion abundance of the quantified PDHC lipids. The mean PDHC abundances and the standard error are depicted for five paired control and atheroma lipid extracts.

FIG. 5 is a dot plot schematically representing the results of PDHC lipid analysis of brain samples obtained from active MS patients and control patients. Frozen brain samples from control patients (n=13) and MS patients with active disease (n=12) were analyzed for the presence of bacteria-originated PE DHC and PG DHC using MRM-MS. The PG DHC/PE DHC total ion abundance ratios were calculated using the summed ion recoveries from pooled HPLC fractions.

FIG. 6 is a dot plot schematically representing the results of PDHC lipid analysis of serum samples obtained from active MS patients and control patients. Serum samples from control (n=16) and MS patients (n=19) were analyzed for the presence of bacteria-originated PE DHC and PG DHC using MRM-MS. PG DHC/PE DHC total ion abundance ratios were calculated using the ion abundances recovered from samples of the total serum lipid extracts.

FIG. 7 is a line plot illustrating enhancement of experimental allergic encephalomyelitis (EAE) by P. gingivalis total lipid (TL) and the PE DHC lipid fraction in female C57BL/6 wild-type (WT) mice aged 4-8 weeks, which were immunized subcutaneously with MOG35-55 peptide (100-200 μg/mouse) in CFA containing 500 μg of H37Ra mycobacteria on day 0. Mice also received Ptx intravenously (150-250 ng) on days 0 and 2. On day 0, mice also received a single 20-μl intraperitoneal (i.p.) injection of EtOH, P. gingivalis TL (2.5 μg), or P. gingivalis PE DHC (250 ng). EAE was graded as follows: grade 1, tail paralysis; grade 2, abnormal gait; grade 3, hind limb paralysis; grade 4, hind and front limb paralysis; grade 5, death. The results illustrated are from one representative experiment each and are depicted as the average EAE score of a given cohort of mice on each day after immunization.

FIG. 8 is a line plot illustrating enhancement of EAE by P. gingivalis total lipid (TL) and the PE DHC lipid fraction in female WT and IL-15−/− mice aged 4-8 weeks, which were immunized subcutaneously with MOG35-55 peptide (100-200 ng/mouse) in CFA containing 500 μg of H37Ra mycobacteria on day 0. Mice also received Ptx intravenously (150-250 ng) on days 0 and 2. On day 0, mice also received a single 20-μl i.p. injection of EtOH, P. gingivalis TL (2.5 μg), or P. gingivalis PE DHC (250 ng). Additional WT mice also received a single 20-μl i.p. injection of the control lipid, bovine sphingomyelin (250 ng). EAE was graded as discussed above. The results illustrated are from one representative experiment each and are depicted as the average EAE score of a given cohort of mice on each day after immunization.

FIG. 9 is a line plot illustrating enhancement of EAE by P. gingivalis total lipid (TL) and the PE DHC lipid fraction in WT and IL-15Ra−/− in female mice aged 4-8 weeks, which were immunized subcutaneously with MOG35-55 peptide (100-200 μg/mouse) in CFA containing 500 μg of H37Ra mycobacteria on day 0. Mice also received Ptx intravenously (150-250 ng) on days 0 and 2. On day 0, mice also received a single 20-μl i.p. injection of EtOH, P. gingivalis TL (2.5 μg), or P. gingivalis PE DHC (250 ng). EAE was graded as discussed above. Results illustrated are from one representative experiment each and are depicted as the average EAE score of a given cohort of mice on each day after immunization.

FIG. 10 is a line plot illustrating that the PE DHC lipid fraction fails to enhance EAE in TLR2−/− mice. EAE was induced and graded as discussed above using wild-type (WT) or TLR2−/− mice. On day 0, wild-type and TLR2−/− mice received a single 20-μl i.p. injection of EtOH or P. gingivalis PE DHC (250 ng). Results illustrated are a composite of studies (WT mice, n=28; TLR2−/− mice, n=15) and represent the average EAE score for each group (±SEM for wild-type mice) on each day after immunization.

FIG. 11 is a plot schematically illustrating the results of electrospray MS analysis of PE DHC lipids recovered from P. gingivalis. Total lipids of P. gingivalis were isolated and fractionated by high performance liquid chromatography (HPLC). Fractions containing the characteristic molecular ions of PE DHC lipids were pooled and repurified by HPLC. Repurified fractions demonstrating 705, 699, and 677 negative ions were pooled. The structure of the high-mass PE DHC lipid (705 m/z) is shown in the inset with the component fatty acid and long-chain base structures identified. The lower-mass PE DHC lipids indicated by 691 or 677 m/z ions contain 18 carbon or 17 carbon long-chain bases, respectively. The plot shows the absence of ions characteristic for lipid A moieties produced by P. gingivalis (1195, 1435, 1449, 1690, and 1770 m/z negative ions).

FIG. 12 is a dot plot, which illustrates the results of the animal study demonstrating that administration of PE DHC resulted in increased recovery of bacterial lipids in the brains of mice with EAE. PBS, EtOH, or PE DHC-injected mice (25 ng, 250 ng, or 2.5 μg) were sacrificed after day 20 post-EAE immunization. The brains of these mice were removed, extracted for phospholipids, and 3-OH isoC_{17:0} fatty acid quantified using negative ion chemical ionization gas chromatography-mass spectrometry. The average 3-OH isoC_{17:0} recovery (three determinations per mouse brain sample) as a function of both the treatment and final EAE score was depicted as picograms of 3-OH isoCl_{7:0} per 0.5 mg of total brain lipid extracted. The average SEM for all brain lipid determinations was ±2.2 pg/0.5 mg total lipid.

FIG. 13 is a bar graph, which illustrates the results of an in vitro study demonstrating that the PE DHC lipid fraction activated APCs and induced IL-6 secretion in vitro in a TLR2-dependent manner. Bone marrow-derived DCs from wild-type (WT) or TLR2−/− mice were cultured alone or with plate-bound EtOH, LPS (1 μg), MMP (10 μg), or PE DHC (2.5 μg). After 18 hours, culture supernatants were assayed for IL-6 via enzyme-linked immunosorbent assay. Histogram bars depict the mean±SD (n=4 trials).

FIG. 14 is a two dimensional dot plot illustrating the data obtained from a flow cytometry analysis which illustrates the results of an in vitro study demonstrating that the PE DHC lipid fraction activated APCs and induced IL-6 secretion in vitro in a TLR2-dependent manner. Naïve CD4+CD25− wild-type Teff (0.25×106/well) were cultured with irradiated wild-type or TLR2−/− Tds (T cell-depleted splenocytes) as a source of antigen presenting cells (0.75×106/well), anti-CD3 antibody (1 μg/ml), granulocyte macrophage-colony-stimulating factor (20 ng/ml), and transforming growth factor-β (2 ng/ml). In addition, LPS (2 μg/ml), MMP (5 μg/ml), or P. gingivalis PE DHC (20 μg/ml as a sonicated liposome preparation) were added to wells to stimulate IL-6 secretion. Cultures were harvested after 5 days, stimulated in culture for 4 hours with phorbol 12-myristate 13-acetate, ionomycin and brefeldin A and stained for Thy1.2, intracellular IFNγ, and IL-17 and analyzed by fluorescence-activated cell sorting after gating on Thy 1.2+ cells.

FIG. 15 contains schematic representations of the chemical structures of L-serine containing lipids. Panel A shows the structure of Lipid 654 and the negative ion fragments as determined by MS/MS. Panel B shows the structure of Lipid 654 and the positive ion mass and fragment ions as determined using the QTrap instrument in the positive ion mode. Panel C shows the structure of Lipid 430 and the negative ion fragment masses as determined by MS/MS.

FIG. 16 is a bar graph showing TLR-2 mediated stimulation by Lipid 654. HEK293 cells, transfected with the human TLR2, CD14, and SEAP (secreted embryonic alkaline phosphatase) genes, were used to assay the function of Lipid 654 in vitro. Stimulation with a TLR2 ligand activates NF-κB and AP-1 which induce the production of SEAP which is then quantitated as a colorimetric change in the presence of a detection medium. HEK293 cells were stimulated for 24 hours with: no stimulation (labeled as “Unstim”); DMSO (vehicle: 50% mixture of DMSO/water); the known TLR2 ligand MMP; the known TLR2 ligand LTA; or Lipid 654. In each case, the cells were stimulated in the presence of no abs, or anti-TLR2ab, or anti-TLR6 ab. Responses were assessed after 24 hours and results expressed as a ratio of stimulated/non-stimulated responses.

FIG. 17 is a bar graph demonstrating that Lipid 654 is not an agonist for TLR4. HEK293 cells, transfected with the human TLR4, CD14, and SEAP (secreted embryonic alkaline phosphatase) genes, were used to assay the function of Lipid 654 in vitro. HEK293 cells were stimulated for 24 hours with: no stimulation (“Unstimulated”); DMSO (vehicle: 50% mixture of DMSO/water); the known TLR2 ligands MMP and LTA; two different preparations of Lipid 654 (“old and “new”); and two different preparations of the known TLR4 agonist, LPS derived from P. gingivalis. Responses were assessed after 24 hours and results expressed as a ratio of stimulated/non-stimulated responses.

FIG. 18 is a bar graph demonstrating TLR2 mediated stimulation by Lipid 654 and Lipid 430. HEK293 cells, transfected with the human TLR2, CD14, and SEAP (secreted embryonic alkaline phosphatase) genes, were used to assay the function of Lipid 654 and Lipid 430 in vitro. HPLC fractions prepared from the total lipid extract were evaluated by ESI-MS and the relative levels of Lipid 654 and Lipid 430 are depicted for the indicated HPLC fractions. The relative TLR2 responses are indicated for each HPLC fraction. TLR2 responses were assessed after 24 hours.

FIG. 19 is a plot showing the levels of Lipid 654 in human serum for control patients and patients with MS. Blood samples were obtained from 12 healthy volunteers and 17 MS patients. Serum lipids were derived from 0.5 ml of the serum samples and analyzed, in three separate determinations, by MRM-mass spectrometry for expression of Lipid 654. The results represent the mean (and standard error) of the absolute ion abundances of Lipid 654 in a representative determination.

FIG. 20 is a graph depicting the ROC curve analysis for the diagnostic use of Lipid 654.

FIG. 21 is a graph showing the results of the effect of Lipid 654 on EAE. Female SJL mice were injected intra-peritoneally with 30×10⁶ 4-day in-vitro-activated PLP-stimulated lymph node lymphocytes and on the same day injected intravenously with either phosphate buffered saline (PBS) (vehicle control=“VC”) or 2 μg of Lipid 654. Mice were followed for 30 days for development of EAE and scored as 1=tail paralysis; 2=abnormal gait; 3=paralyzed hind legs; 4=paralyzed front and hind legs; 5=death. Results depict the daily mean for 5 mice injected with the VC and 5 mice injected with Lipid 654; p=0.0006, Mann-Whitney test.

FIG. 22 depicts the results showing a significant difference between serum and carotid artery samples (p<0.0001; Mann-Whitney test). The mean Lipid 430/Lipid 654 ratio increased in carotid artery walls by greater than three orders of magnitude over serum levels.

FIG. 23 is a graph depicting the results of Lipid 654 hydrolysis to Lipid 430 by the following enzymes: porcine pancreatic phospholipase A2 (PP PLA2), honey bee venom PLA2, bovine liver nonspecific esterase (BLE), phospholipase C (PLC), lipoprotein lipase (LL), phospholipase D (PLD), and cobra venom factor (CVF). The ratios of Lipid 654 to 430 were determined. The results are shown as the Log (10) values.

FIG. 24 contains graphs showing that Lipid 654 is significantly lower in the serum of MS patients. Serum was obtained from MS patients and healthy individuals. Total serum lipids were derived from these samples and analyzed using MRM-mass spectrometry to identify and quantify the absolute ion abundance of three transitions of Lipid 654 (Transitions 1, 2 and 3). Ion abundance is expressed as 10⁵. MS patients, N=17; healthy individuals, N=12. Wilcoxon's rank-sum test was used to determine statistical significance.

FIG. 25 shows the results of the addition of an internal standard to the MRM-mass spectrometry analysis, which confirms that Lipid 654 is significantly lower in the serum of MS patients. Serum was obtained from MS patients and healthy individuals and total serum lipids were analyzed by MRM-mass spectrometry for expression of Lipid 654 using Transitions 1, 2 and 3 as in FIG. 24. As an additional control for MRM-mass spectrometry efficiency, a defined quantity of ¹³C-labeled total lipids derived from P. gingivalis was added to each sample. The level of recovery of ¹³C-labeled Lipid 654 was then used to adjust each value based on the efficiency of the analysis of that sample. Ion abundance is expressed as 10⁵. MS patients, N=17; healthy individuals, N=12. Means: Transition 1: MS patients 414,891; control patients 3,003,525; Transition 2: MS patients 66,482; control patients 497,055; Transition 3: MS patients 2,762; control patients 211,245. Wilcoxon's rank-sum test was used to determine statistical significance.

FIG. 26 shows that Lipid 654 expression is lower in the serum of MS patients versus Alzheimer's patients. Frozen banked serum samples from MS and Alzheimer's patients were obtained. Total serum lipids were derived and analyzed using MRM-mass spectrometry to identify and quantify the absolute ion abundance of Lipid 654 using three transitions of Lipid 654 (Transitions 1, 2 and 3, as in FIG. 24). As in FIG. 25, a defined quantity of ¹³C-labeled total lipids derived from P. gingivalis was added to each sample and used to adjust each value based on the efficiency of the analysis of that sample. Ion abundance is expressed as 10⁵. MS patients, N=13; Alzheimer's patients, N=15. Means: Transition 1: MS patients 112,139; Alzheimer's patients 1,297,909; Transition 2, MS patients 26,333; Alzheimer's patients 295,680; Transition 3: MS patients 13,786; Alzheimer's patients 142,076. Wilcoxon's rank-sum test was used to determine statistical significance.

FIG. 27 shows the level of HEK293 activation by lipids recovered in HPLC fractions of P. gingivalis total lipids. HEK293 cells, transfected with the human TLR2, MD-2, CD14, and SEAP genes, were used to assay the functions of P. gingivalis lipid fractions in vitro. A defined volume of each HPLC fraction was dried and reconstituted in 50% DMSO in water. The final concentration of DMSO achieved in culture medium was 1.11%. HEK293 cells were stimulated for 24 hours with a defined amount of each lipid fraction. Results are expressed as the stimulated/nonstimulated (DMSO control) response ratio of HEK293 cells (graph A). Graphs B and C show the ion abundances within each HPLC fraction of lipids that produced negative ions of m/z 654 and 430, respectively.

FIG. 28 shows the MS/MS profiles of 654, 640, and 626 lipid species. The partial mass spectra are depicted for the m/z 654 lipid species (A and D), the m/z 640 lipid species (B and E), and the m/z 626 lipid species (C and F). The structure of the most abundant species in the lipid 654 class is shown in graph G.

FIG. 29 shows the MS/MS profiles of 430 lipid species. The partial mass spectra are depicted for the m/z 430 lipid species recovered from HPLC-fractionated total lipids of P. gingivalis (A), NaOCH₃-treated lipid 654 (B), or KOH-treated lipid 654 (C). The structure shown in panel D represents the deesterified form of the 654 lipid species shown in FIG. 28. See the FIG. 28 legend for reconciliation of the low-mass product ions (<200 amu).

FIG. 30 shows the HEK cell activation by lipid classes derived from P. gingivalis. HEK293 cells, transfected with the human TLR2, MD-2, CD14, and SEAP genes, were used to assay the function of P. gingivalis lipid classes in vitro. HEK293 cells were stimulated for 24 h with the following treatments: DMSO (vehicle; 50% mixture of DMSO-water, approximately 1.11% DMSO in the final culture medium; n=23), the known TLR2 ligand MMP (0.2 μg/ml; n=34), LTA (2 μg/ml; n=17), lipid 654 (0.17 μg/ml [n=5]; 0.34 μg/ml [n=2]; 0.69 μg/ml [n=22]), lipid 430 (0.17 μg/ml [n=2]; 0.34 μg/ml [n=3]; 0.69 μg/ml [n=18]), subPG-DHC (0.69 μg/ml; n=4), unPG-DHC (0.69 μg/ml; n=4), PE-DHC (0.69 μg/ml; n=4), and PEA (0.69 μg/ml; n=9). The phospholipid preparations were prepared from P. gingivalis total lipids. Responses were assessed after 24 hours, and results are expressed as the ratio of stimulated versus nonstimulated (DMSO) responses. By one-way ANOVA and Fisher LSD pairwise comparisons, HEK cell activation levels by MMP, LTA, lipid 654, and lipid 430 (both at 0.69 μg/ml) were significantly elevated over the DMSO vehicle (P<0.05).

FIG. 31 is a graph showing the TLR2-mediated stimulation levels by lipid 654 and lipid 430. HEK293 cells, transfected with the human TLR2, MD-2, CD14, and SEAP genes, were used to assay the function of lipid 654 and lipid 430 in vitro. For antibody blocking, cells were preincubated for 1 hour with neutralizing anti-human TLR2 antibody (10 μg/mL; InvivoGen). HEK293 cells were stimulated for 24 hours with DMSO (vehicle; 50% mixture of DMSO-water; approximately 1.11% DMSO in final culture medium; n=4), the known TLR2 ligand MMP (0.1 μg/ml; n=6), the known TLR2 ligand LTA (1 μg/ml; n=6), lipid 430 (0.69 μg/ml; n=2), and lipid 654 (0.69 μg/ml; n=7). The sample sizes (n) for each treatment refer to the number of both untreated and TLR2-blocked samples. Responses were assessed 24 hours after the addition of TLR2 ligands, and results are expressed as the ratio of stimulated to nonstimulated (DMSO) responses.

FIG. 32 contains a graph showing that Lipid 654 and lipid 430 are not agonists for TLR4. HEK293 cells, transfected with the human TLR4, CD14, MD-2, and SEAP genes, were used to assay the function of lipid 654 and lipid 430 in vitro. HEK293 cells were stimulated for 24 hours with DMSO (vehicle; 50% DMSO in water), the known TLR2 ligands MMP and LTA, lipid 654 (0.69 μg/ml), lipid 430 (0.69 μg/ml), and two different preparations of known TLR4 agonists, LPS derived from Salmonella enterica or P. gingivalis (1 μg/ml). Responses were assessed at 24 hours, and results are expressed as the ratio of stimulated to nonstimulated (DMSO) responses (n=2).

FIG. 33 shows the results of the in vivo administration of lipid 654 or lipid 430 to wild-type and TLR2^{-/-} mice. In Panel (A), Lipid 654 (1 μg) or vehicle (50% mixture of DMSO-water) was injected i.p. into either WT or TLR2^{-/-} mice. Four hours later, the mice were bled and the sera assayed for levels of CCL2 by ELISA. Histogram bars represent the mean±standard error of the mean (SEM) for 3 or 4 trials. In Panel (B), Lipid 430 (2.5 μg) or vehicle (PBS) was injected i.v. into either WT or TLR2^{-/-} mice. Four hours later, the mice were bled and the sera assayed for levels of CCL2 by ELISA. Histogram bars represent the mean±SEM for 3 trials. Statistical significance was assessed using the Student t test, with symbols indicating the following significance levels: *, P=0.0164; #, P=0.0012 for WT versus TLR^{-/-} lipid responses.

### DETAILED DESCRIPTION

The subject matter of embodiments of the present invention is described here with specificity to meet statutory requirements, but this description is not necessarily intended to limit the scope of the claims. The claimed subject matter may be embodied in other ways, may include different elements or steps, and may be used in conjunction with other existing or future technologies. This description should not be interpreted as implying any particular order or arrangement among or between various steps or elements except when the order of individual steps or arrangement of elements is explicitly described.

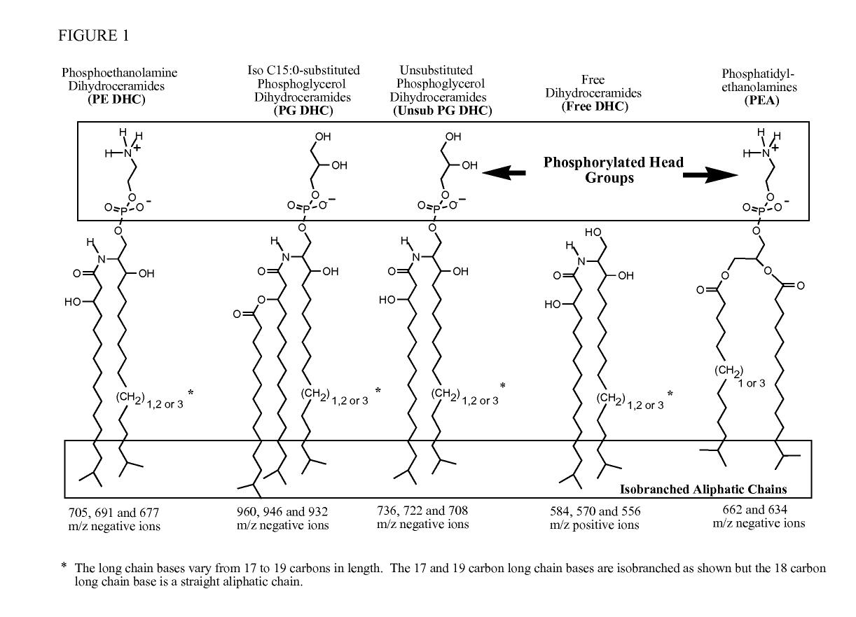

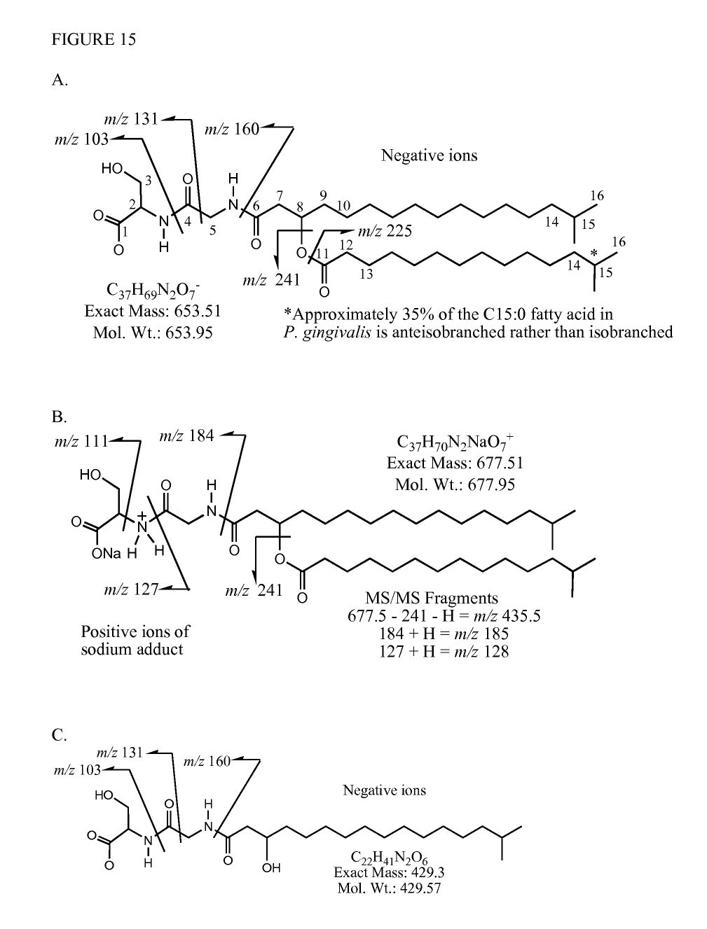

Some embodiments of the present invention utilize, in a novel and unexpectedly beneficial way, information on bacterial lipids in humans or animals. In particular, some of the embodiments of the present invention utilize information on occurrence of bacterial lipids in a human or an animal in a novel and unexpectedly way that is indicative of an inflammatory or an autoimmune disease or a condition in the human or the animal. Bacterial lipids utilized in the relevant embodiments of the present invention are synthesized by pathologic or non-pathologic bacteria found in a human or an animal organism but not synthesized by the organism itself. These lipids may be referred to as “bacteria-originated” lipids. In one exemplary embodiment, bacteria-originated lipids are bacterial phosphorylated dihydroceramides (PDHCs), biologically active lipids, unique to bacteria, which are capable of promoting inflammatory reactions in human cells in vitro, as described, for example, in Nichols, et al., “Prostaglandin E2 secretion from gingival fibroblasts treated with interleukin-1 beta: effects of lipid extracts from Porphyromonas gingivalis or calculus.” J. Periodontal. Res. 36(3):142-52 (2001), and Nichols, et al. (2004). Two major classes of biologically active lipids are found in PDHCs: phosphoethanolamine dihydroceramide (PE DHC) and phosphoglycerol dihydroceramide (PG DHC) schematically illustrated in FIG. 1. These lipids, integral parts of the bacterial membranes, are likely released upon the death or phagocytosis/endocytosis of the organism. In another exemplary embodiment, bacteria-originated lipids are bacterial L-serine containing lipids. The L-serine containing lipids described herein are unique to bacteria and are biologically active. These lipids mediate significant effects on the innate immune system. Examples of L-serine containing lipids include Lipid 654 and Lipid 430, where the numerical designation in the names of the lipids refer to the most abundant negative ion mass as determined by mass spectrometry. Lipid 654 and Lipid 430 are schematically illustrated in FIG. 15A-C. It has been discovered that these lipids are produced by many bacteria found commonly in the oral cavity and gastrointestinal tract of human or animal organisms, and can be recovered from the serum, gingiva, and brains of human or animal organisms. Optionally, the human or animal organisms are healthy human or animal organisms (e.g., non-multiple sclerosis (MS) human or animal organisms. Optionally, Lipid 654, Lipid 430, or both are isolated from the oral cavity or gastrointestinal tract of a human or animal organism (e.g., a healthy human or animal organism). It is to be understood that the term “bacteria originated lipid” or “bacteria originated lipids” are used herein to refer to lipids derived from bacteria, for example, isolated by various isolation techniques, as well as to substantially similar molecules synthesized or generated under laboratory or industrial conditions.

However, the relevant embodiments of the present invention are not intended to be limited by PDHCs and L-serine containing lipids. Rather, any lipid can be used in the embodiments of the present invention, as long as the information on their occurrence, used alone or in combination with other information, is indicative of an inflammatory or autoimmune disease or conditions. Some of the bacterial lipids used in the embodiments of the present invention may alter the physiology of mammalian lipids, resulting in disease-related alterations in the presence or levels of mammalian lipids in human tissues including the blood. Some embodiments of the present invention utilize bacteria-originated lipids, or lipids comprising structures not produced by mammals, allowing them to be specifically identified in mammalian tissue using various analytical techniques, such as negative ion electrospray mass-spectrometry and multiple reaction monitoring mass-spectrometry (MRM-MS).

As discussed above, bacterial lipids utilized in the methods of the present invention generally originate in bacteria inhabiting human and animal bodies and organisms. Some of these bacterial are habitual inhabitants and are often referred to as “commensal” bacteria, particularly when they are not associated with any pathological states or conditions. Some other of the bacterial are described as “pathological,” particularly if they are typically not found in human or animal organisms, or found in low numbers, and their presence or increased numbers is associated with a pathological state. It is noted that the same bacterial species can be classified as both “commensal” or “pathological,” depending on the accepted classification system, pathology paradigm, bacterial numbers, and other factors. The present invention is therefore not limited to the uses of the lipids originating from commensal, pathological, or any other category of bacteria. Some non-limiting examples of the bacterial lipids used in the methods described herein originate in Bacteroides or Prevotella, Porphyromonas, Tannerella, Prevotella and Parabacteroides genera of bacteria. For example, Lipid 654, Lipid 430, or both can originate in Porphyromonas gingivalis, a periodontal pathogen. Optionally, Lipid 654, Lipid 430, or both can be isolated from Porphyromonas gingivalis for use in the methods described herein.

One embodiment of the present invention provides a method for detecting an inflammatory or an autoimmune condition, comprising analyzing or detecting bacterial lipids in a sample; and, comparing results of the analysis of the bacterial lipids in the sample with information on occurrence of the bacterial lipids in a comparable sample, wherein the comparison is indicative of the inflammatory or the autoimmune condition. A sample can be obtained from a human or an animal. The method for detecting an inflammatory or an autoimmune condition can further comprise, prior to the step of analyzing, obtaining by any suitable method, such as extracting, a lipid fraction from the sample. The step of analyzing can comprise one or more of: identifying the bacterial lipids; quantitating the bacterial lipids; or determining one or more quantitative relationship among categories of the bacterial lipids detected during the analysis. The information on occurrence of the bacterial lipids can include information on one or more quantitative relationship among categories of the bacterial lipids.

In some of the embodiments, the bacterial lipids analyzed in the method discussed above are PDHCs, including phosphoethanolamine dihydroceramides (PE DHCs) and phosphoglycerol dihydroceramides (PG DHCs). In some of the embodiments, the analysis involves determining the ratio of total ion abundance of PG DHC to PE DHC. In one exemplary embodiment, the methods described herein use a ratio of PG DHC to PE DHC as indicative of MS. In one example, an increased ratio of PG DHC to PE DHC in a blood sample, as compared to a control blood sample, indicates the presence of MS. Optionally, the control blood sample is obtained from a normal or non-MS human subject. Optionally, the control blood sample is obtained from a human subject diagnosed with MS during a period when the subject's disease activity is low.

In other embodiments, the bacterial lipids analyzed in the method discussed above are L-serine containing lipids, including Lipid 654 and/or Lipid 430. Optionally, the analysis involves measuring the serum levels of Lipid 654 or Lipid 430 in a subject and comparing the levels to serum levels obtained from a healthy human subject (e.g., a non-MS human subject). In one example, decreased levels of Lipid 654 and/or Lipid 430 in serum, as compared to a control serum sample, indicates the presence of MS. Optionally, the control serum sample is obtained from a normal or non-MS human subject. Optionally, the control serum sample is obtained from a human subject diagnosed with MS during a period when the subject's disease activity is low. Not to be bound by theory, a deficit in Lipid 654 or Lipid 430 in a subject can have a role in the cause of MS.

Optionally, Lipid 654 and/or Lipid 430 can be used to inhibit experimental allergic encephalomyelitis (EAE). Optionally, Lipid 654 and/or Lipid 430 can be used to treat MS patients. The methods of treating patients with Lipid 654 and/or Lipid 430 can include administering to the patient an effective amount of Lipid 654 and/or Lipid 430. Optionally, Lipid 654 and/or Lipid 430 can be administered directly to the patient (i.e., isolated Lipid 654 and/or isolated Lipid 430, optionally provided in a composition in combination with other pharmaceutically acceptable ingredients) can be administered to the patient). Optionally, Lipid 654 and/or Lipid 430 can be administered by administering to the patient the commensal bacteria that produce increased amounts of Lipid 654.

As described herein, bacteria-originated lipids, such as PDHCs and L-serine containing lipids, that originate from bacteria found in multiple sites in humans (gingiva, GI tract and vagina), possess previously unknown immunomodulating properties. Accordingly, the present invention encompasses compositions or medicaments comprising bacteria-originated lipids, which are useful for modulating or affecting immune responses, as well as uses and methods of using bacteria-originated lipids to modulate immune responses in a human or an animal. In some exemplary embodiments, compositions, uses and methods induce or exacerbate an autoimmune or an inflammatory state in a human or an animal. Such embodiments can be useful for research or diagnostic purposes, for example, for creation of animal models or for observation of an autoimmune disease flare-up in a patient. However, compositions, uses and methods that decrease or alleviate an autoimmune or an inflammatory state in a human or an animal are also envisioned and fall within the scope of the present invention. According to some embodiments of the present invention, compositions comprising bacteria-originated lipids contain PE DHC. Corresponding methods of use or uses involve PE DHC-containing compositions. According to some embodiments of the present invention, compositions comprising bacteria-originated lipids contain L-serine containing lipids. Corresponding methods of use or uses involve compositions including L-serine containing lipids.

Some other embodiments of the present invention include compositions comprising bacteria-originated lipids, which affect toll-like receptor (TLR) pathways and activities. In one embodiment, compositions according to some embodiments of the present invention comprise a TLR-receptor ligand. Optionally, the bacteria-originated lipids function as a ligand for TLR2. Corresponding methods and uses of such compositions are also included in the scope of the present invention. For example, methods of using such compositions to activate a TLR receptor or a TLR receptor signaling pathway or response are included. Methods that involve binding of a TLR ligand disclosed herein to a TLR receptor for research or diagnostic purposes, such detection of a TLR receptor, are also included in the scope of the embodiments of the present invention. The terms “signaling pathway” or “signaling response” are used in reference to biological processes conventional known as “signaling” which generally involve a molecule binding to and activating a protein known as a “receptor”, which, in turn, affects other molecules, thus generating a so-called signaling response, cascade or pathway. The term toll-like receptors (TLRs) is used herein in a conventional manner to refer to a class of proteins that are currently known to play an important role in the innate immune system, and to generally recognize structurally conserved molecules derived from microbes.

The term “composition,” as used herein, encompasses compositions of matter, chemical, analytical, pharmaceutical, therapeutic, preventive or diagnostic compositions, biologically, pharmacologically, immunologically or immunochemically active compositions. The term “composition” also includes medicaments, drugs, medicines, pharmaceuticals, reagents, such as analytical reagents. The term “compositions” encompasses compositions that include one component or ingredient, as well as compositions including more than one component or ingredient. Compositions can comprise both “active” and “inactive” ingredients or components. The term “active” as used herein in reference to a component or ingredient of a composition (which can also be denoted as an “agent”) refers to a compound that possesses an activity relevant to the use of the composition. As used herein, the term “effective amount” can refer to an amount of an active agent that exhibits an activity relevant to the use of the compositions. Effective amounts vary with various uses, durations, other included into the compositions, and other factors. It is to be understood that any of the components of the compositions according to the embodiments of the present invention that are denoted as inactive agents, explicitly or by implication, nevertheless can change the activity of the active agents, and can also have independent effects of inactivating other inflammatory processes. As used herein, the term “effective amount” can also refer to an amount of an inactive agent that exhibits an activity relevant to the use of the compositions. The term “method” as used herein encompasses methods of using and uses of compositions according to various embodiments of the present invention.

The terms “detect,” “detecting,” “indicate,” “indicative” and similar terms are used in this document to broadly refer to a process or discovering or determining the presence or an absence, as well as a degree, quantity, or level, or probability of occurrence of something. For example, the term “detecting” when used in reference to a disease or a condition can denote discovery or determination one or more of presence of a disease or a condition, absence of a disease or a condition, progression, level or severity of a disease or a condition, as well as a probability of present or future exacerbation of symptoms, or of efficacy of a treatments. The foregoing list is not intended to be exhaustive, and the terms “detect,” “detecting,” “indicate,” “indicative” and similar can also refer to other things.

The terms “analysis” or “analyzing” and similar terms are used herein to broadly refer to studying or determining a nature, properties, or quantity of an object under analysis, or its components. Analysis can include detection, as discussed above. Analysis can also involve chemical or biochemical manipulations or steps, as well as manipulations or steps of other nature, as well as manipulation of information in an appropriate manner (for example, storage of information in computer memory and computer calculations may be used).

The term “occurrence” when used in reference to bacterial lipids utilized in some of the embodiments of the present invention is used to denote incidence of the bacterial lipids, as well as frequency of their appearance, quantity, or distribution throughout different classes or subclasses. In some embodiments of the present invention, any of the foregoing information falling within the meaning of the term “occurrence” can be utilized in relation to one or more bacterial lipids, as well as classes and subclasses of such lipids. The combination of such information on the occurrence of lipids can be referred to as “pattern” or “lipid pattern.” The information on occurrence of bacterial lipids, or lipid patterns, obtained in the course of performing the methods described herein can be compared or correlated with the information previously obtained, processed or stored. The results of such comparison, according to certain embodiments of the present invention, lead to detection of a disease or a condition. When the information on occurrence of bacterial lipids is derived from a sample obtained from a human or an animal patient, the method is useful for detection of a disease or a condition in the patient. The methods of the present invention can utilize bacterial lipids, including bacteria-originated lipids, as markers, biomarkers, or biological markers to detect a disease or a condition, such as autoimmune or inflammatory disease or condition. In some embodiments, the methods described also include detecting a predisposition to develop a disease or condition at some future time. In other words, the occurrence of bacterial lipids can be used in the present invention as a characteristic measured and evaluated as an indicator of certain biological processes. These processes may include autoimmune diseases, such as Rheumatoid Arthritis and Systemic Lupus Erythematosus, and generalized vascular disease, as it occurs in atherosclerosis.

The analysis of bacterial lipids used in the methods of the present invention can involve various analytical techniques suitable for qualitative or quantitative detection of lipids, including, but not limited to HPLC, gas chromatography, mass-spectrometry, immunochemical techniques and assays (ELISA), and lipid arrays (described, for example, in U.S. Patent Publication US20070020691).

The term “condition” when used in reference to the embodiments of the invention disclosed herein is used broadly to denote a biological state or process, such as an immune or inflammatory response, which can be normal or abnormal or pathological. The term “condition” can be used to refer to a medical or a clinical condition, meaning broadly a process occurring in a body or an organism and distinguished by certain symptoms and signs. The term “condition” can be used to refer to a disease or pathology, meaning broadly an abnormal disease or condition affecting a body or an organism.

Some conditions detected by the detection methods disclosed herein are inflammatory or autoimmune conditions. Non-limiting examples or autoimmune conditions are rheumatoid arthritis, systemic lupus erythematosus (SLE), diabetes (type 1) or multiple sclerosis (MS). Non-limiting examples of inflammatory conditions are periodontal disease or atherosclerosis.

As used herein, the terms “multiple sclerosis” or “MS” refer to a disease or condition that affects the brain and spinal cord (central nervous system) of humans and can exhibit any of the symptoms described below. While MS is currently characterized in the medical field as a condition arising out of autoimmune damage to the myelin sheath, the embodiments of the present invention are not limited by this characterization and encompass detection of MS-like diseases and conditions that are broadly encompassed by the clinical criteria described below, even if these diseases and conditions have causes, origins or mechanisms different from those covered by the presently accepted MS paradigm. MS is most commonly diagnosed between ages 20 and 40, but can be observed or diagnosed at any age. MS symptoms vary, and the location, severity and duration of each MS attack can be different. Episodes can last for days, weeks, or months and alternate with periods of reduced or no symptoms, generally referred to as remissions. It is common for MS to relapse, but it also may continue without periods of remission. MS patients can have any of the following symptoms, in various combinations: muscle symptoms, which include loss of balance, muscle spasms, numbness or abnormal sensation in any body area, problems moving arms or legs, problems walking, problems with coordination and making small movements, tremor in one or more arms or legs or weakness in one or more arms or legs; bowel and bladder symptoms, which include constipation and stool leakage, difficulty initiating urination, frequent need to urinate, strong urge to urinate, urine leakage (incontinence); eye symptoms, which include double vision, eye discomfort, uncontrollable rapid eye movements, vision loss (usually affects one eye at a time); numbness, tingling, or pain; facial pain; painful muscle spasms; tingling, crawling, or burning feeling in the arms and legs; other brain and nerve symptoms, which include decreased attention span, poor judgment, and memory loss, difficulty reasoning and solving problems, depression or feelings of sadness, dizziness and balance problems, hearing loss; sexual symptoms; speech and swallowing symptoms, which include slurred or difficult-to-understand speech, trouble chewing and swallowing; and/or fatigue.

The terms “sample” or “samples,” as used interchangeably herein, refer to any cell or tissue samples or extracts originating from human or animal subject, and include samples of human or animal cells or tissues as well as cells of non-human or non-animal origin, including bacterial samples. A sample can be directly obtained from a human or animal organism, or propagated or cultured. Samples can be subject to various treatment, storage or processing procedures before being analyzed according to the methods described herein. Generally, the terms “sample” or “samples” are not intended to be limited by their source, origin, manner of procurement, treatment, processing, storage or analysis, or any modification. Samples include, but are not limited to samples of human cells and tissues, such as blood samples, cerebrospinal fluid samples, synovial tissue samples, synovial fluid samples, brain tissue samples, blood vessel samples, or tumor samples. Blood samples include both blood serum and blood plasma samples. Samples encompass samples of healthy or pathological cells, tissues or structures. Samples can contain or be predominantly composed of bacterial cells. The terms sample or samples can refer to the samples of structures or buildup commonly referred as plaques, such as atheromatous plaque, dental plaque, senile plaque, mucoid, and dermal plaque. Some examples of samples are blood plasma or blood serum samples, including the samples from periodontally healthy subjects, blood plasma or blood serum samples from subjects with generalized severe destructive periodontal disease, such as chronic periodontitis, subgingival microbial plaque samples, carotid atheroma samples and tissue samples derived from human brain. Some other examples of samples are samples of teeth, skin, or kidneys.

In one of its embodiments, the present invention provides a lipid-specific antibody capable of specific binding to a PDHC lipid category, such as an antibody capable of specific binding with PG DHC or PE DHC. Antibodies described herein are useful for detecting a PDHC lipid in a sample, for modulating an immune response in a human or animal cell or tissue or in a human or an animal organism, and can be incorporated into pharmaceutical compositions and medicaments for modulating immune responses. Antibodies described herein can also be useful in diagnostic methods, such as detection methods according to some other embodiments of the present invention described herein. Antibodies described herein are also useful for detecting a PDHC lipid in a sample and can be incorporated into diagnostic kits and reagents.

In other embodiments, the present invention provides a lipid-specific antibody capable of specific binding to an L-serine containing lipid category, such as an antibody capable of specific binding with Lipid 654 or Lipid 430. Antibodies described herein are useful for detecting an L-serine containing lipid in a sample, for modulating an immune response in a human or animal cell or tissue or in a human or an animal organism, and can be incorporated into pharmaceutical compositions and medicaments for modulating immune responses. Antibodies described herein can also be useful in diagnostic methods, such as detection methods according to some other embodiments of the present invention described herein. Antibodies described herein are also useful for detecting an L-serine containing lipid in a sample and can be incorporated into diagnostic kits and reagents.

The terms “modulating,” “modulation,” and similar terms, when used in reference to immune responses and pathways (which can also be denoted as “immunomodulating”), inflammatory responses and pathways, as well as TLR responses and pathways are used generally to refer to modification of immune responses, processes and cascades in response to a modulating agent, such as an antibody Immunomodulation can result in an increased immune response or a decreased immune response, or both an increase and a decrease, when assessed through different parameters or processes. The term “immune response” encompasses the whole scope of animal immune response, including innate and adaptive immunity.

Based on the results from the methods of detecting an inflammatory or an autoimmune condition, a practitioner or care provider can start, stop, or modify the treatment of the subject. In some embodiments, where a test sample from an individual indicates an increased blood serum or blood plasma ratio of PG DHC to PE DHC as compared to a control (e.g., a non-MS individual), the practitioner or care provider can begin treating the individual with therapeutic agents, such as anti-inflammatory agents or agents to treat multiple sclerosis. In some embodiments, where a test sample from an individual indicates an altered level of blood serum or blood plasma Lipid 654, Lipid 430, or both as compared to a control (e.g., a non-MS individual), the practitioner or care provider can begin treating the individual with therapeutic agents, such as anti-inflammatory agents or agents to treat multiple sclerosis.

The compositions according to some embodiments of the present invention can be readily formulated with, prepared with, or administered with, a pharmaceutically acceptable carrier. Such preparations may be prepared by various techniques. Such techniques include bringing into association active components of the compositions and an appropriate carrier. In one embodiment, compositions are prepared by uniformly and intimately bringing into association active components of the compositions with liquid carriers, with solid carriers, or with both. Liquid carriers include, but are not limited to, aqueous formulations, non-aqueous formulations, or both. Solid carriers include, but are not limited to, biological carriers, chemical carriers, or both.

The compositions according to some embodiments of the present invention may be administered in an aqueous suspension, an oil emulsion, water in oil emulsion and water-in-oil-in-water emulsion, and in carriers including, but not limited to, creams, gels, liposomes (neutral, anionic or cationic), lipid nanospheres or microspheres, neutral, anionic or cationic polymeric nanoparticles or microparticles, site-specific emulsions, long-residence emulsions, sticky-emulsions, micro-emulsions, nano-emulsions, microspheres, nanospheres, nanoparticles and minipumps, and with various natural or synthetic polymers that allow for sustained release of the composition including anionic, neutral or cationic polysaccharides and anionic, neutral cationic polymers or copolymers, the minipumps or polymers being implanted in the vicinity of where composition delivery is required. Polymers and their use are described in, for example, Brem et al., Journal of Neurosurgery 74:441-446 (1991). Furthermore, the active components of the compositions according to some embodiments of the present invention can be used with any one, or any combination of, carriers. These include, but are not limited to, anti-oxidants, buffers, and bacteriostatic agents, and may include suspending agents and thickening agents.

For administration in a non-aqueous carrier, active components of the compositions according to some embodiments of the present invention may be emulsified with a mineral oil or with a neutral oil such as, but not limited to, a diglyceride, a triglyceride, a phospholipid, a lipid, an oil, and mixtures thereof, wherein the oil contains an appropriate mix of polyunsaturated and saturated fatty acids. Examples include, but are not limited to, soybean oil, canola oil, palm oil, olive oil, and myglyol, wherein the number of fatty acid carbons is between 12 and 22 and wherein the fatty acids can be saturated or unsaturated. Optionally, one or more charged lipids or phospholipids can be suspended in the neutral oil. More specifically, use can be made of phosphatidylserine, which targets receptors on macrophages. Use can be made of active components of the compositions according to embodiments of the present invention formulated in aqueous media or as emulsions using techniques known to those of ordinary skill in the art.

The compositions according to some embodiments of the present invention can comprise active agents described elsewhere in this document, and, optionally, other therapeutic and/or prophylactic ingredients. The carrier and other therapeutic ingredients must be acceptable in the sense of being compatible with the other ingredients of the composition and not deleterious to the recipient thereof.

The compositions according to some embodiments of the present invention are administered in an amount effective to induce a therapeutic response in an animal, including a human. The dosage of the composition administered will depend on the condition being treated, the particular formulation, and other clinical factors such as weight and condition of the recipient and route of administration. In one embodiment, the amount of the composition administered corresponds from about 0.00001 mg/kg to about 100 mg/kg of an active component per dose. In another embodiment, the amount of the composition administered corresponds to about 0.0001 mg/kg to about 50 mg/kg of the active component per dose. In a further embodiment, the amount of the composition administered corresponds to about 0.001 mg/kg to about 10 mg/kg of the active component per dose. In another embodiment, the amount of the composition administered corresponds to about 0.01 mg/kg to about 5 mg/kg of the active component per dose. In a further embodiment, the amount of the composition administered corresponds to from about 0.1 mg/kg to about 1 mg/kg of the active component per dose.

Useful dosages of the compounds of the present invention can be determined by comparing their in vitro activity and in vivo activity in animal models. Methods for the extrapolation of effective dosages in mice, and other animals, to humans are known in the art; for example, see U.S. Pat. No. 4,938,949.

Modes of administration of the compositions used in the invention are exemplified below. However, the compositions can be delivered by any of a variety of routes including: by injection (e.g., subcutaneous, intramuscular, intravenous, intra-arterial, intraperitoneal), by continuous intravenous infusion, cutaneously, dermally, transdermally, orally (e.g., tablet, pill, liquid medicine, edible film strip), by implanted osmotic pumps, by suppository, or by aerosol spray. Routes of administration include, but are not limited to, topical, intradermal, intrathecal, intralesional, intratumoral, intrabladder, intravaginal, intra-ocular, intrarectal, intrapulmonary, intraspinal, dermal, subdermal, intra-articular, placement within cavities of the body, nasal inhalation, pulmonary inhalation, impression into skin, and electroporation.

Depending on the route of administration, the volume of a composition according to some embodiments of the present invention in an acceptable carrier, per dose, is about 0.001 ml to about 100 ml. In one embodiment, the volume of a composition in an acceptable carrier, per dose is about 0.01 ml to about 50 ml. In another embodiment, the volume of a composition in an acceptable carrier, per dose, is about 0.1 ml to about 30 ml. A composition may be administered in a single dose treatment or in multiple dose treatments, on a schedule, or over a period of time appropriate to the disease being treated, the condition of the recipient, and the route of administration. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

### EXAMPLES

Embodiments of the present invention are illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the invention. During the studies described in the following examples, conventional procedures were followed, unless otherwise stated. Some of the procedures are described below for illustrative purpose.

Procurement, Storage and Processing of Bacterial Samples

Bacterial samples previously stored frozen at −80° C. in skim milk were grown on blood agar plates after demonstrating purity of bacterial isolates. Bacteria were identified by 16 S rRNA sequencing (˜1400 bp). Phenotypic tests were done when needed to fully identify an organism. The plates were scraped to recover the bacterial colonies and were extracted using the phospholipid extraction procedure described in Bligh, E. G. & Dyer, W. J. “A rapid method of total lipid extraction and purification.” Can. J. Biochem. Physiol. 37: 911-917 (1959), as modified by the procedures described in Garbus, J. et al. “Rapid incorporation of phosphate into mitochondrial lipids” J. Biol. Chem. 238:59-63 (1968). Porphyromonas gingivalis (type strain, ATCC#33277), Tannerella forsythia and Prevotella intermedia (VPI 8944) were grown in broth culture and after pelleting bacteria by centrifugation, the bacterial pellets were stored frozen until processing. P. gingivalis and P. intermedia were grown in broth culture according to the procedures described, for example, in Nichols et al., “Prostaglandin E₂ release from monocytes treated with lipopolysaccharides isolated from Bacteroides intermedius and Salmonella typhimurium: Potentiation by gamma interferon” Infect. Immun. 59:398-406 (1991), and Nichols, F. C. & Rojanasomsith, K. “Porphyromonas gingivalis lipids and diseased dental tissue”. Oral Microbiol. Immunol. 21:84-92 (2006). At the time of lipid extraction, samples of bacterial pellets were removed and extracted using the phospholipid extraction procedure discussed above.

Procurement, Storage and Processing of Human Samples

PDHC Lipid Samples:

Human tissue and blood plasma and blood serum samples were obtained according to conventional procedures and guidelines. All tissue and blood samples were stored frozen until processing. Human tissue samples were stored frozen (−20° C.) until the time of lipid extraction. Atheroma samples were processed as follows. The patent segment of the common carotid artery (control samples) was excised from the grossly apparent atheroma of the carotid body, and PDHCs in the lipid extracts from the individual paired samples were quantified. The patent carotid artery samples showed no apparent gross atheroma formation though these artery segments were partially calcified within the artery wall. Gingival tissue, atheroma and brain samples were thawed and at least 20 mg of tissue was minced and extracted for several days in organic solvent, according the method of Bligh & Dyer (1959). After drying organic solvent extracts under nitrogen, the lipid extracts were reconstituted in hexane:isopropanol:water (HPLC solvent, 6:8:0.75, v/v/v), vortexed and centrifuged. The resultant supernatants were recovered, a sample of defined volume (5 μl) was dried and weighed, and a defined amount of each sample was transferred to a clean vial either for further processing or for MRM-MS analysis. For brain samples, 10 mg of each lipid extract was fractionated by normal phase HPLC as described in Nichols et al. (2004). The fractions expected to contain the PDHC lipids were pooled and dried. Each brain lipid isolate was then reconstituted in 300 μl of HPLC solvent and 5 μl was analyzed by MRM-MS for the bacterial lipids of interest. For each subgingival plaque sample, 50 μg of lipid extract was dissolved in 200 μl of HPLC solvent and 5 μl of each sample was analyzed by MRM-MS. For gingival tissue samples, 1 mg of lipid extract was dissolved in 300 μl of HPLC solvent and 5 μl of each sample was analyzed by MRM-MS. Citrated blood samples, obtained by venipuncture from periodontal patients, were diluted 2:1 (v/v) in saline and subjected to Ficoll-Hypaque centrifugation. Plasma samples were aspirated following centrifugation and stored frozen until lipid extraction. For lipid extraction, the plasma samples were thawed and 0.5 ml of each sample was extracted for lipids as described above. The dried lipid samples were reconstituted in 300 μl of HPLC solvent and analyzed by MRM-MS.

L-Serine Containing Lipid Samples:

Lipid 654 preparations were recovered according to the methods described above. Lipid 430 preparations were recovered using the procedures described above with the following modifications: Lipid 430 was extracted from aqueous solvent where the pH was reduced to less than 3.5, which was accomplished by adding acetic acid and extracting the sample with chloroform.

Analysis of Lipid Samples

Individual lipid samples were analyzed using a 4000 QTrap 4000 mass spectrometer (AB Sciex®, Foster City, Calif.). A standard volume of each lipid sample (5 μl) was analyzed by flow injection and HPLC solvent was run at a rate of 80 μl/min. Using previously purified lipid preparations of each phosphorylated dihydroceramide class, the instrument parameters were optimized for detection of each lipid component based on gas phase transitions depicted in FIG. 1. Standard curves were generated using serially diluted lipid standards of known quantity and linearity of lipid quantification was observed (regression coefficients >0.99). In addition, carryover of individual lipid ion transitions into other monitored transitions was not observed. Using the optimized instrument parameters, each lipid extract from tissue, blood and bacterial samples was individually analyzed. Each lipid ion transition peak was electronically integrated and the percentage abundance of each lipid class was calculated from the integrated lipid ion transition peaks. For each category of tissue or blood samples, all samples within a particular tissue or blood category were analyzed during a single analysis session. Two-factor ANOVA or the paired student t test was used to test for significant differences between sample categories.

Mice

Female C57BL/6 (WT) mice were obtained from Jackson Labs (Bar Harbor, Me.). TLR2^{-/-} mice were a generous gift of Dr. S. Akira (Osaka University, Japan), IL-15^{-/-} mice and IL-15Rα^{-/-} mice were a generous gift from Dr. Leo LeFrancois (University of Connecticut Health Center). All mice were maintained and bred in accordance with conventional animal care procedures.

Induction of Experimental Allergic Encephalomyelitis (EAE)

EAE served as a murine model of MS. Female mice (4-8 weeks old) were immunized with 100-200 μg of myelin oligodendrocyte glycoprotein peptide (35-55) (MOG) emulsified with CFA (containing 500 μg of H37RA mycobacteria) (DIFCO Co—BD Diagnostics, Sparks, M D) via a subcutaneous (s.c.) injection on Day 0. 200-250 ng of Pertussis toxin (List Biologicals Labs, Campbell, Calif.) was injected intravenously (i.v.) on Day 0 and again on Day 2. In addition, mice were injected intraperitoneally (i.p.) on Day 0 with either P. gingivalis lipid or the vehicle control, 70% ethanol (EtOH). EAE was scored as: Grade 1-tail paralysis; Grade 2-weakness of hind limbs with an altered gait; Grade 3-hind limb paralysis; Grade 4-front limb paralysis; Grade 5-death.

Purification and Verification of P. gingivalis Lipids

P. gingivalis (ATCC#33277, type strain) was grown and lipids extracted and fractionated by HPLC as previously described in Nichols et al. (2004); Nichols “Novel ceramides recovered from Porphyromonas gingivalis: relationship to adult periodontitis” J. Lipid Res. 39:2360-2372 (1998). HPLC fractions highly enriched for PE DHC lipids were identified via electrospray-MS using a Micromass Quattro II mass spectrometer system as described in Nichols et al. (2004). HPLC fractions containing highly enriched PE DHC lipids were pooled and each combined fraction was verified to be of greater than 95% purity by electrospray-MS.

Processing of Lipids for Administration to Animals and Addition to Tissue Culture

For treatment of mice, preweighed lipids were dissolved in 70% ethanol to achieve a final concentration of 125 ng/μl, and sonicated for 2.5 minutes immediately before injection into experimental animals. This preparation was also used for drying lipids onto tissue culture wells. For direct addition to cell cultures, the lipids were dissolved in culture medium at 125 ng/μl and sonicated for 2.5 minutes to produce a liposome preparation for administration to cells in culture.

Derivation and Stimulation of Bone Marrow Dendritic Cells (DCs)

Bone marrow cells from C57BL/6 and TLR2^{-/-} mice were cultured at 2×10⁵ cells/ml in RPMI containing 10% FCS, 2-ME, and 20 ng/ml recombinant murine GM-CSF for 9 days. Bone marrow DCs (BMDCs) were harvested at Day 9 and were greater than 80% CD11c+. LPS (1 μg), MMP (10 μg) (a bacterial lipoprotein and known TLR-2 ligand (Bachem H-9460), PE DHC (2.5 μg) or 70% EtOH, all in 20 ul volumes, were allowed to dry in the wells of a 24-well plate overnight prior to the addition of BMDCs. BMDCs were cultured in the ligand-bound 24-well plates at 1×10⁶ cells/ml in RPMI containing GM-CSF. After 18 hrs, culture supernatants were harvested and tested for IL-6 via ELISA.

In Vitro Generation of Th17 T Cells

CD4+CD25− T cells (Teff) were derived from WT mice using magnetic bead purification (Miltenyi Biotec, Auburn, Calif.). T cell-depleted splenocytes (Tds) were derived from WT or TLR2^{-/-} mice using magnetic bead purification followed by irradiation (2600R). Teff (0.25×10⁶/well) and Tds (0.75×10⁶/well) were cultured in 24-well plates with anti-CD3 antibody (1 μg/ml), GM-CSF (20 ng/ml) (Pierce Inc., Thermo Fisher Scientific, Rockford, Ill.) and recombinant TGF-β (2 ng/ml) (R&D). In addition, LPS (2 μg/ml), MMP (5 μg/ml) or P. gingivalis PE DHC (20 μg/ml of sonicated liposome preparations) were added to wells to stimulate the secretion of IL-6. Cultures were harvested after 5 days, stimulated in culture for 4 hrs with phorbol myristyl acetate and ionomycin and stained for Thy1.2, intracellular IFNγ, and IL-17 and analyzed by FACS after gating on Thy 1.2+ cells.

Derivation and Phenotypic Analysis of Spinal Cord-Derived Mononuclear Cells

Spinal cord mononuclear cells were derived as previously described in Korn et al. “Myelin-specific regulatory T cells accumulate in the CNS but fail to control autoimmune inflammation” Nat. Med. 13:423-431 (2007) and stained for CD4 (FITC α-CD4 (GK1.5) BD Pharmingen) and Foxp3 (APC α-FoxP3 (FJK-16s; E-Bioscience), or stimulated in culture for 4 hrs with phorbol myristyl acetate and ionomycin prior to staining for Thy1.2 (PE-Cy7 anti-CD 90.2; E-Biosciences) and intracellular IFNγ (APC αIFNγ; BD Pharmingen) and IL-17 (Alexa Fluor 488 α-IL-17A; BD Pharmingen).

Recovery of Bacterial Lipids from the Brains of Mice with EAE

Mice treated with PBS, EtOH or with 25 ng, 250 ng, or 2.5 μg PE DHC were sacrificed after day 20 post-EAE immunization. The brains were removed and extracted for phospholipids according to the method of Bligh and Dyer as previously described in Nichols “Distribution of 3-hydroxy iC17:0 in subgingival plaque and gingival tissue samples: Relationship to adult periodontitis” Infect. Immun. 62:3753-3760 (1994). Lipid extracts were dissolved in hexane isopropanol:water (6:8:0.75, v/v/v/) and three 0.5 mg aliquots were dispensed into glass tubes supplemented with 30 ng of isobranched C_{20:0}. Lipid samples were hydrolyzed for 4 hours in 2N KOH, acidified and fatty acids extracted into chloroform and dried. Lipids were treated to form pentofluorobenzyl ester, trimethylsilyl ether derivatives and analyzed by negative ion chemical ionization GC-MS, as described in Nichols (1994). Fatty acid recovery was quantified by selected ion monitoring for characteristic fatty acid negative ions. The data were expressed as picograms of 3-OH isobranched (iso)C_{17:0} per 0.5 mg of total brain lipid extracted.

Statistical Procedures for EAE Animal Model Studies

The cumulative disease index (CDI) was obtained by summing the daily average disease scores through Day 20. A mean of these daily disease scores (Mean Daily Disease) (+/−SEM) was calculated based on the 20 days of observation. The Mean Daily Disease scores were compared using the Wilcoxin Signed Rank tests for two samples. Disease incidence frequencies were compared using Chi square analysis. Values for mean maximum severity of EAE were compared using the Wilcoxin Signed Rank test. Values for mean day of onset of EAE were compared using the Student's t-test. For analysis of spinal cord populations, percentages were compared using Student's t test. Bacterial fatty acid levels in brain lipid extracts for each treatment group were evaluated using least squares linear regression analysis that included calculation of correlation coefficients. For each dose of bacterial lipid administered, linear regression analysis compared the final EAE score with the mean bacterial fatty acid recovered per 0.5 mg of brain lipid extract. The mean bacterial fatty acid levels were calculated from three replicate brain lipid determinations.

### Example 1

### Lipid Analysis of Bacterial Species from Human Isolates

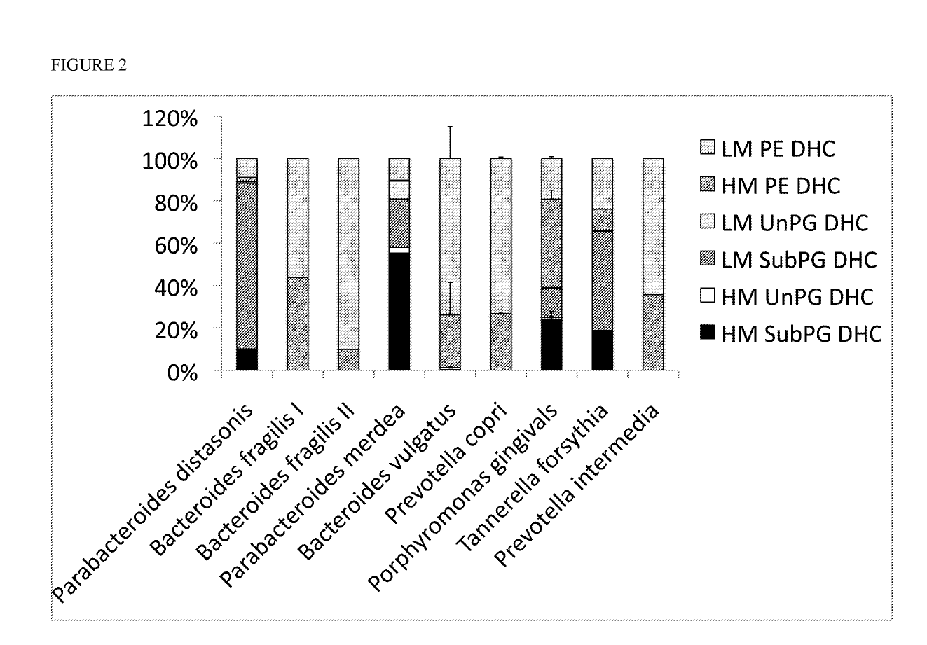

Lipid extracts from 95 intestinal bacterial species from a total of 247 individual human isolates were analyzed. The results of the analysis are schematically represented in FIG. 2. As illustrated in FIG. 2, the lipid analysis revealed that these species varied in their capacity to produce either PE DHC or PG DHC and also varied in their production of the high mass (HM) versus the low mass (LM) forms of these PDHCs. For example, the PDHC lipid constituents produced by P. gingivalis were predominantly HM PE DHC lipids whereas T. forsythia produces primarily LM PG DHC forms.

The lipid analysis of the intestinal and oral bacterial species demonstrated that different strains of the same intestinal species may produce PDHCs with different levels of PG DHC or PE DHC. Intestinal bacteria assessed in the analysis exhibited a tendency to produce primarily PE DHC or PG DHC, but not both. Of the intestinal and periodontal organisms observed to produce PG DHCs, only B. merdea produced a small amount of the unsubstituted (“UnPG DHC”) lipids (˜11% of total PDHC), whereas the remaining intestinal and oral bacteria produced negligible amounts of UnPG DHC lipids.

The lipid analysis of the intestinal and oral bacterial species showed that they varied in their capacity to produce specific PDHC lipids, and that the combinations of intestinal and oral bacterial organisms have the ability to deposit unique mixtures of PDHCs in human tissues.

### Example 2

### Lipid Analysis of Human Samples

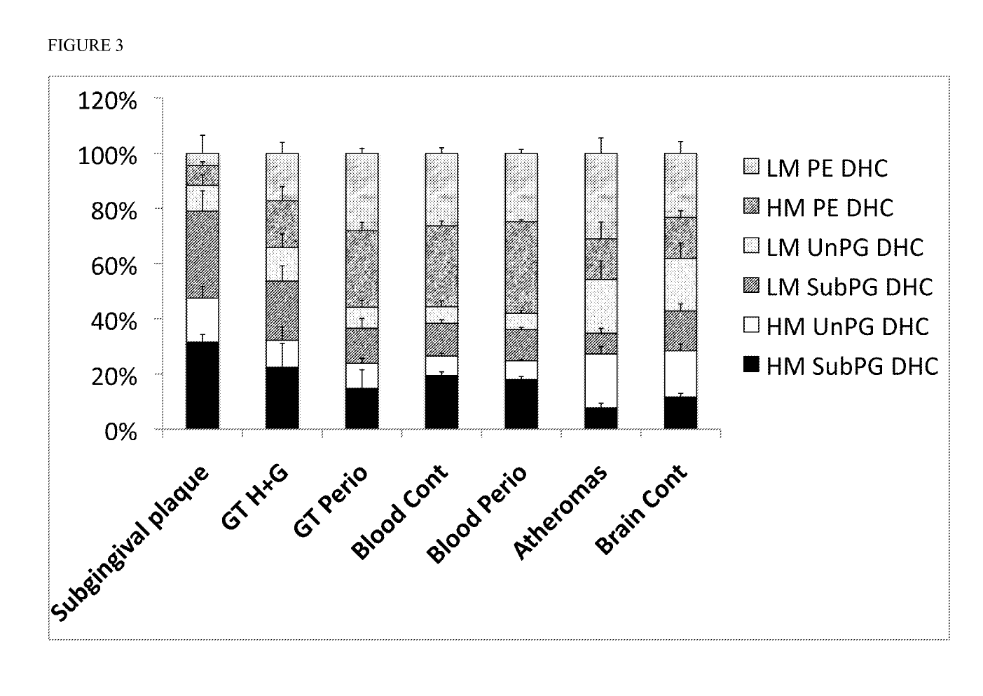

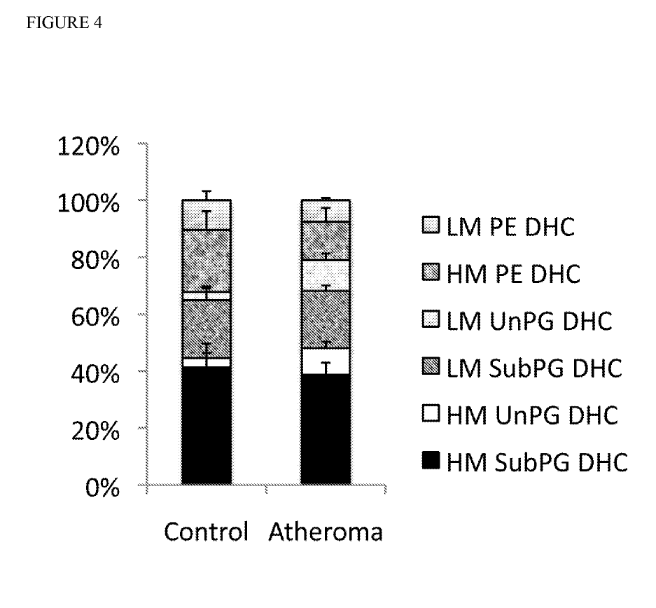

The results of the lipid analysis of human samples are schematically represented in FIGS. 3 and 4. The following samples obtained from human subjects were analyzed: subgingival plaque samples (2 samples) healthy/mildly inflamed gingival tissue (GT H+G, 7 samples), periodontitis gingival tissue samples (GT Perio, 6 samples), control blood plasma samples from periodontally healthy subjects (Blood Cont, 8 samples), blood plasma from patients with generalized severe periodontitis (Blood Perio, n=7), carotid atheroma (Atheroma, n=11) and postmortem brain samples from non-MS subjects. Deposition of PDHCs was observed in all of the human tissue samples examined. The distribution of PDHCs in the examined tissue samples showed distinctive patterns. PDHCs detected in human tissue samples were a mixture of HM and LM forms and revealed significant percentages of both LM or HM UnPG DHC lipids. Comparative analysis of blood plasma samples from periodontally healthy subjects and subjects with chronic periodontitis revealed substantial percentages of both LM or HM UnPG DHC lipids. Analysis of lipid extracts from atheroma artery segments revealed higher percentages of HM or LM UnPG DHC, when compared with the control artery extracts. The total ion abundances of PDHC lipids per μg of total lipid extract were 33 times higher on average in the control artery segments than the atheroma segments. Lipid extracts of brain samples showed a mean percentage of UnPG DHC lipids comparable to or higher than those observed in carotid atheromas. In contrast, subgingival microbial plaque samples taken from gingival crevices at periodontitis sites showed only minimal levels of UnPG DHC. Comparative analysis of PDHC lipids in healthy versus inflamed (periodontitis) gingival tissue and associated blood plasma samples was performed. Two-factor ANOVA revealed significantly lower percentages of HM and LM SubPG DHC lipids and significantly higher percentages of HM and LM PE DHC lipids in periodontitis gingival tissue samples versus healthy samples. Similarly, blood plasma samples demonstrated a significant increase in the percentage of HM PE DHC lipids in periodontitis plasma versus healthy plasma samples, while SubPG DHC percentages were not lower in plasma samples from periodontitis patients. The analysis showed that shifts in the deposition of specific bacterial lipids (PE DHCs) in gingival tissues was directly correlated with expression of destructive periodontal disease and that this specific increase in PE DHC is also reflected in blood plasma levels. In control carotid samples, atheroma samples, and in brain samples, the percentages of PG DHC lipids were relatively higher than that in both blood samples and diseased gingival. Analysis of deposition of PDHC in human tissues showed that distribution patterns of bacterial PDHCs in human tissues correlate with health and disease states. In particular, distribution patterns of bacterial PDHCs in human tissues correlate with inflammation states.

### Example 3

### Analysis of Brain Tissue Samples from MS Patients with Active Disease

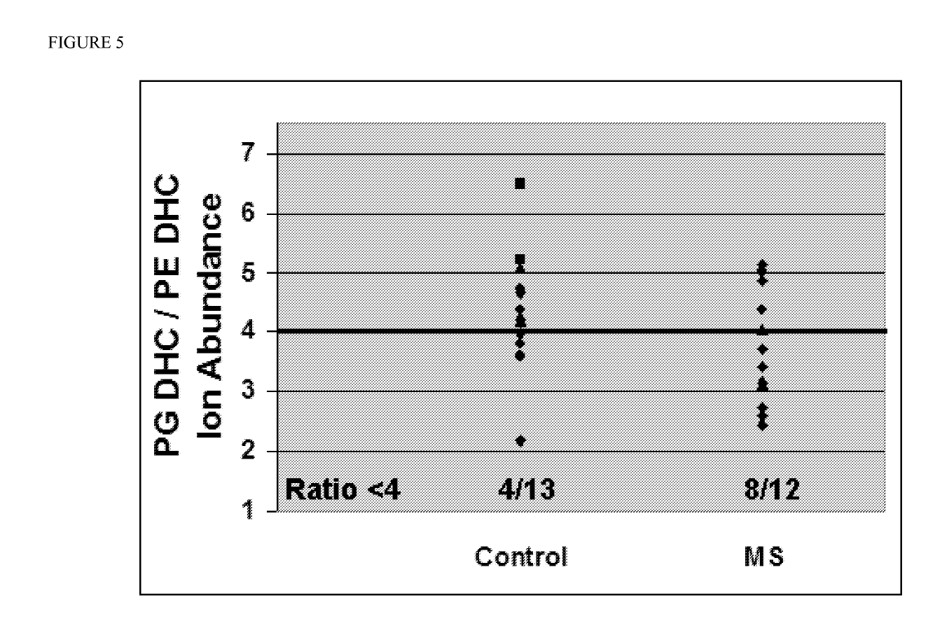

Analysis of coded (blinded) frozen brain samples obtained from control subjects and from MS patients with active disease was performed. The results of the analysis are schematically represented in FIG. 5, which shows the PG DHC/PE DHC ion abundance ratios of 13 control and 12 active MS brain samples. The samples were analyzed for the presence of bacteria-originated PE DHC and PG DHC lipids using MRM-MS. The MRM-MS approach was somewhat different from the MRM-MS approach utilized in the study described in Example 2. Following analysis of the samples by MRM-MS, the ratio of PG DHC to PE DHC, measured as total ion abundance, was calculated. It was observed that all brain samples analyzed contained some level of PDHCs. Quantification of the different PDHC classes from control and active MS patients demonstrated surprising results. While the absolute levels of PE DHC and PG DHC were not statistically different between control and active MS patients (using two factor ANOVA), the proportional recovery of these fractions was different. A higher level of PE DHC together with a slightly lower (or unchanged) level of PG DHC was found in brain samples from active MS patients versus controls (both healthy and other neurological disease (OND) patients), resulting in an MS-specific PDHC lipid pattern. A decrease in mean PG DHC/PE DHC ratios was thus observed in MS brain samples. While the decrease in mean PG DHC/PE DHC ratios did not reach statistical significance, only 31% of control samples (4/13), but 67% of MS samples (8/12), showed a PG DHC/PE DHC ratio of less than 4.0 using this specific analytic approach. While mean ratios differed between control and MS brain samples at the p=0.09 level (unpaired Student's t-Test), one control sample was an outlier, demonstrating a ratio of 2.18 (over 2.2 standard deviations from the mean). If this outlier was removed, the mean brain PG DHC/PE DHC ratios for MS versus controls differed significantly, with p=0.02 (unpaired Student's t-Test). The lipid analysis of brain samples from MS patients described in this example showed that the presence of MS in a patient correlated with a decrease in the PG DHC/PE DHC ratio, measured as ion abundance or mean ratio.

### Example 4

### Analysis of Serum Samples from MS Patients with Active Disease

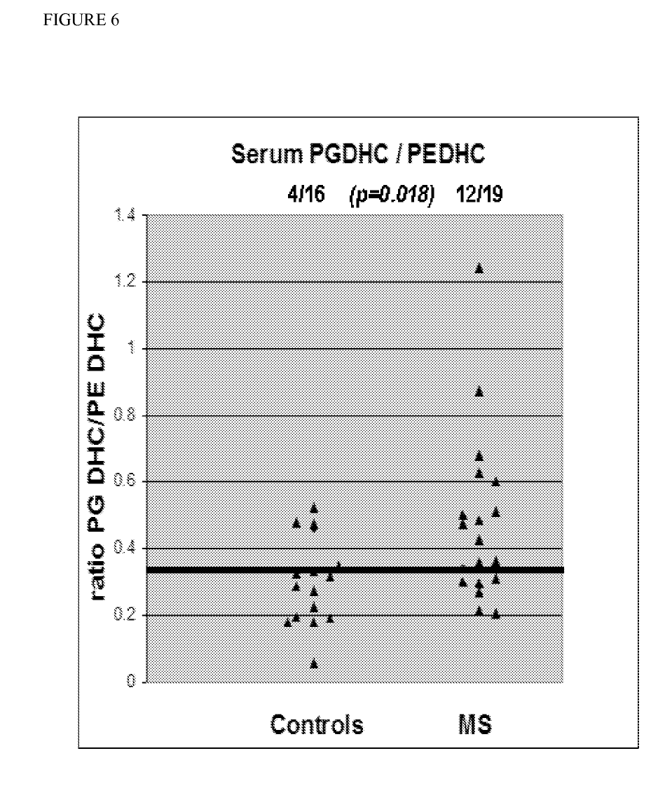

The results of PDHC analysis of serum samples from MS patients with active disease is schematically illustrated in FIG. 6. Serum samples were obtained from a group of healthy control patients and from a group of MS patients (“MS samples”). The MS patients included both genders, a wide age distribution, and represented different MS subtypes and therapeutic treatments. Control samples were obtained from patients that had no acute or chronic health problems, included both genders, and had an age distribution substantially similar to the group of MS patients. Lipids were extracted from the samples and analyzed for the presence of bacteria-originated PE DHC and PG DHC using MRM-MS. In the studies described in this example, as compared to those described in Example 2, serum rather than plasma samples were examined. The analysis of the serum samples involved a somewhat different MRM-MS approach than the approach used in the studies described in Example 2. A group of 19 MS and 16 control samples was analyzed for levels of PDHCs. Ratios of PG DHC to PE DHC total ion abundance were used to compare PDHCs in control vs. MS samples. Statistically significant differences (using several statistical approaches) were found between PDHC levels in control and MS samples. PE DHC levels were decreased, PG DHC levels were similar, and PG DHC/PE DHC ratios were increased in MS versus control samples. Using two factor ANOVA, it was found that the mean absolute ion abundance of PE DHCs (per 5 μg of total serum lipid extract) was statistically significantly lower in MS patients (mean=46,159+/−SEM of 11,360) than in controls (mean=71,684+/−SEM of 7,276). Thus, the absolute amount of PE DHC per 5 μg of total serum lipid extract was significantly lower in MS versus control samples using both Scheffe contrasts among pairs of means (p<0.05) and Fisher LSD (p=0.0006). The mean level of total lipids derived from control serum samples was not significantly different from MS serum samples. Serum PG DHC levels were not significantly different between MS and control samples; thus PG DHC levels served as an “internal reference” for shifts in PE DHC levels. Using this “internal reference,” it was discovered that PG DHC/PE DHC ratios were significantly different between control and MS serum samples. Mean PG DHC/PE DHC ratios were significantly higher in MS serum (0.478+/−SEM of 0.058) versus control serum (0.300+/−SEM of 0.033) (p=0.018, unpaired Student's t-Test). Furthermore, 63% of MS samples (12/19) had ratios greater than 0.35 while only 25% of control patients (4/16) had ratios greater than 0.35 (see FIG. 6). The results showed no obvious correlation with gender, age, MS subtype, or treatment. The test results using serum PG DHC/PE DHC ratios yielded a diagnostic sensitivity of 63% and a specificity of 75% for MS versus controls.

### Example 5

### PDHC Ratios Correlate with the Presence of MS

The comparative analysis of the brain and serum samples described in the prior examples revealed that PE DHC levels are decreased and PG DHC/PE DHC ratios increased in MS sera samples as compared to the control samples, while the reverse pattern was observed in the MS brain samples as compared to the control samples. The experimental results described in the prior examples showed that distribution of PDHCs in tissues and organs, such as blood and brain, correlated with the presence of MS.

### Example 6

### Bacteria-Originated Lipid Patterns in Human Tissues Indicating Autoimmune or Inflammatory Disease or Condition in a Patient

Tissue samples, such as serum samples, are obtained from patients suffering from an inflammatory condition and/or an autoimmune disease. Analysis of the samples for bacteria-derived lipids, such as PE DHCs, is performed. One of the approaches used in the analysis is MRM-MS, which is capable of specific identification and quantification of the lipid families. The distribution patterns of bacteria-derived lipids in the sample are determined and correlated with one or more of the presence of a disease, the stage or activity of the disease, the efficacy of treatment of the disease. The analysis involves assessments of sub-sets samples taking into account one or more of such factors as gender; age; stage and clinical symptoms of the disease, or treatment status of a patient. Reasonably matched control subjects are used. The analysis reveals patterns of bacteria-originated lipids correlating with presence and status of autoimmune or inflammatory disease or condition in a patient. The patterns are used as diagnostic patterns indicative of an autoimmune or inflammatory disease or condition.

### Example 7

### Bacteria Lipid and Population Patterns Indicating Autoimmune or Inflammatory Disease or Condition in a Patient

Samples of commensal intestinal and oral bacteria are obtained from patients suffering from an inflammatory or an autoimmune disease. Bacterial samples are stored and/or cultured as appropriate to obtain sufficient quantity of bacterial for lipid analysis. Analysis of the bacteria-derived lipids, such as PE DHCs, is performed. One of the approaches used in the analysis is MRM-mass spec, which is capable of specific identification and quantification of the lipid families. The distribution patterns of bacteria-derived lipids in the sample are determined and correlated with one or more of the presence of an autoimmune disease in a patient, the stage or activity of the disease, the efficacy of the treatment of the disease. The analysis involves assessments of sub-set samples taking into account one or more of such factors as gender; age; stage and clinical symptoms of an autoimmune disease, or treatment status of a disease. Reasonably matched control subjects are used. The analysis reveals patterns of bacterial lipids and populations correlating with presence and status of autoimmune or inflammatory disease or condition in a patient. The patterns are used as diagnostic patterns indicative of an autoimmune or inflammatory disease or condition.

### Example 8

### Lipid-Specific Antibodies

Lipid-specific antibodies are prepared that specifically react with various PDHC lipid families (PG DHC and PE DHC). Lipid-specific monoclonal antibodies are prepared as follows: PG DHC and PE DHC are conjugated to immune carriers, such as KLH. Mice are immunized with the resulting conjugates. The sera obtained from the immunized mice are tested by ELISAs for binding to PE DHC and PG DHC which have been conjugated to an irrelevant protein carrier. When the sera are positive, splenocytes from the corresponding mice are fused to an appropriate tumor line to generate hybridoma that secrete antibodies to PE DHC or PG DHC. These (uncloned) hybridoma are tested for binding in the ELISA as above, followed by cloning (by limiting dilution) of any hybridoma showing positive antibodies in the ELISA. These subclones are tested for secretion of antibodies that bind either PE DHC or PG DHC, but not both lipids. Continued subcloning of the hybridomas is conducted as necessary to obtain hybridomas that secrete antibodies binding either PE DHC or PG DHC, but not both lipids. Lipid-specific antibodies are used in immunochemical assays, such as ELISA, to rapidly and easily test serum samples for the presence of lipids of interest.

In an alternative approach for immunizing mice for the purpose of generating monoclonal antibodies, liposomes are generated using, for example, Lipid 654 or Lipid 430 (see G. R. Matyas et al., Journal of Immunological Methods 2000, 245:1-14; G. R. Matyas et al., Journal of Immunological Methods 2002, 267:119-129). In addition to either Lipid 654 or Lipid 430, Lipid A is added to the mixture of lipids that is incorporated into the liposomes. The Lipid 654 or Lipid 430, along with the Lipid A, are resuspended in phosphate buffered saline and sonicated for one minute to generate the liposomes. The resulting liposomes are then injected intraperitoneally (i.p.) into Balb/C mice. Such mice are similarly injected two to five additional times with these liposomes at intervals of two weeks prior to their splenocytes being harvested for generation of monoclonal antibody-producing hybridoma.

### Example 9

### P. gingivalis Total Phosphorylated Dihydroceramides Lipids, and Specifically the PE DHC Fraction, Enhanced EAE

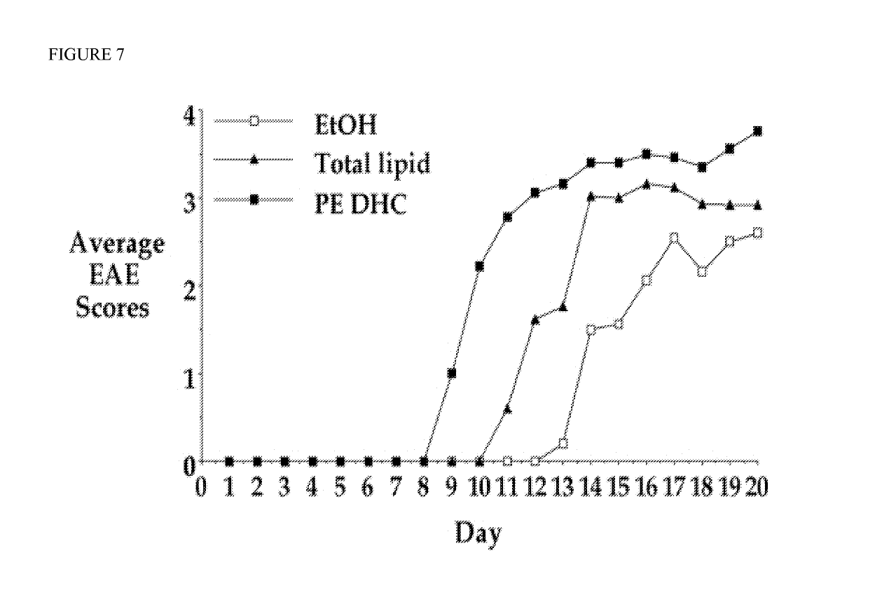

EAE was induced in female C57BL/6 (WT) mice and these mice were also injected i.p. on Day 0 with either P. gingivalis lipids or the vehicle control, 70% ethanol (EtOH). To most effectively detect effects of P. gingivalis lipids in the development of autoimmunity, less severe EAE was induced by using CFA with higher concentrations of H37RA mycobacteria (500 μg/mouse). The effect of administering the P. gingivalis total phosphorylated dihydroceramide lipids (TL) on EAE in wild-type mice was examined A single i.p. injection of 2.5 μg of P. gingivalis TL resulted in enhanced severity of EAE, as illustrated in FIG. 7. Component HPLC fractions of the TL were examined individually. The examination showed that the fraction containing greater than 95% PE DHC most consistently enhanced EAE. Administering 2.5 μg, 250 ng, and even 25 ng of PE DHC led to enhanced disease, with 250 ng being the most efficient. A single 250 ng i.p. injection of the PE DHC fraction consistently enhanced the severity of EAE and often led to earlier onset of disease. FIG. 7 illustrates one representative experiment of six similar studies, in which 250 ng of PE DHC was administered to WT mice. The cumulative results from these six experiments demonstrated that PE DHC-treated mice showed essentially a doubling in cumulative disease index (CDI) and mean daily disease compared with EtOH-treated mice, as illustrated by Table 1. In addition, WT PE DHC-treated mice showed a significantly earlier onset of disease when compared to WT EtOH-treated mice (p=0.008). While not reaching statistical significance, PE DHC-treated mice also showed an increase in incidence of disease, as illustrated in Table 1. Mean maximum severity did not differ significantly between the groups. Of note, the lipids were also administered to naïve mice that were not treated with the EAE-inducing protocol and these mice were observed for signs of illness. Such mice never demonstrated EAE.

### Example 10

### PE DHC Enhances EAE in IL15−/− and IL-15Rα−/− Mice

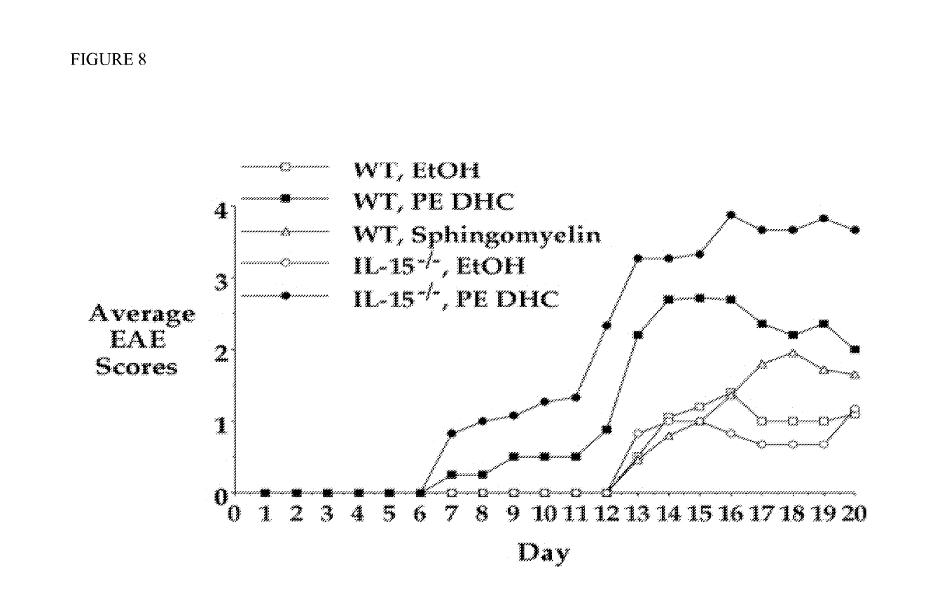

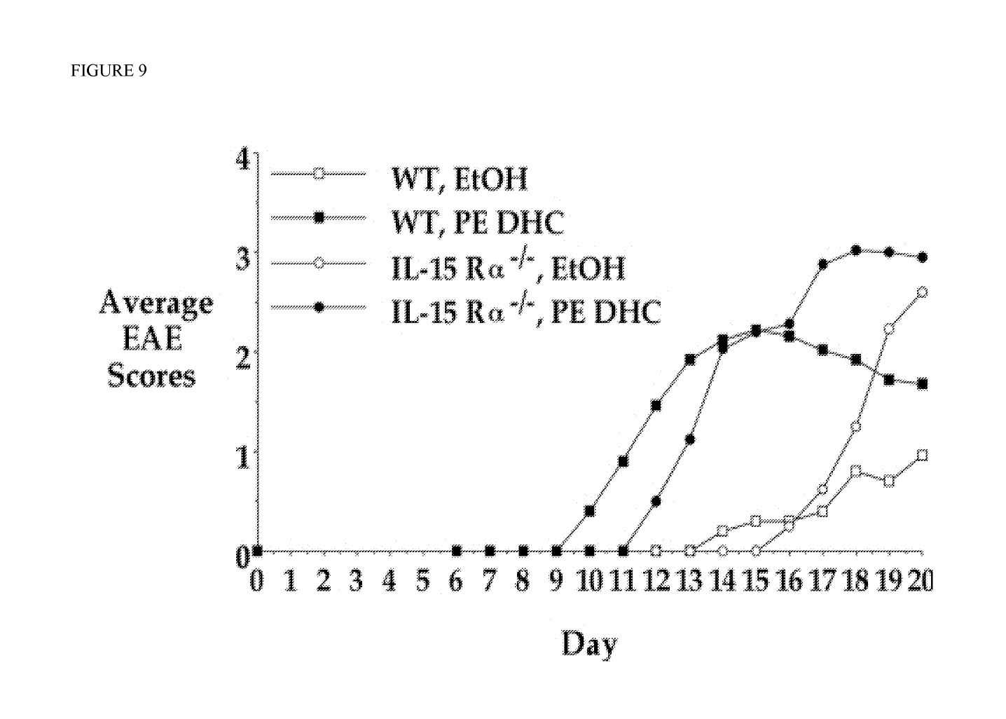

Mice deficient in either IL-15 (IL-15^{-/-} mice) or the IL-15 receptor α (IL-15Rα^{-/-} mice) are known to express very few identifiable NKT cells (Kennedy et al. “Reversible defects in natural killer and memory CD8 T cell lineages in interleukin 15-deficient mice,” J. Exp. Med. 191:771-780 (2000); Lodolce et al. “IL-15 receptor maintains lymphoid homeostasis by supporting lymphocyte homing and proliferation” Immunity 9:669-676 (1998). WT mice and either IL-15^{-/-} or IL-15Rα^{-/-} mice (both on a C57BL/6 background) were immunized for EAE and given a single i.p. injection of EtOH or PE DHC on Day 0. As in WT mice, PE DHC significantly enhanced EAE in both IL-15^{-/-} and IL-15Rα^{-/-} mice, inducing greater than a doubling of the CDI and mean daily disease compared with EtOH-treated IL-15^{-/-} and IL-15Rα^{-/-} mice (as shown Table 1). Additionally, IL-15^{-/-} and IL-15Rα^{-/-} PE DHC-treated mice showed an earlier onset of disease and increased incidence of disease compared to EtOH-treated mice, though only incidence of disease in IL-15Rα^{-/-} PE DHC-treated versus IL-15Rα^{-/-} EtOH-treated mice reached statistical significance (p=0.0285; as shown in Table 1). As with WT mice, mean maximum severity did not differ between the groups. FIGS. 8 and 9 show representative experiments using IL-15^{-/-} and IL-15Rα^{-/-} mice. The finding that PE DHC enhances EAE in IL-15^{-/-} and IL-15Rα^{-/-} mice indicates that PE DHC does not require NKT cells, the most common immune cells known to respond to sphingolipids, in order to mediate its disease-enhancing effect.

### Example 11

### PE DHC Enhancement of EAE is TLR2-Dependent

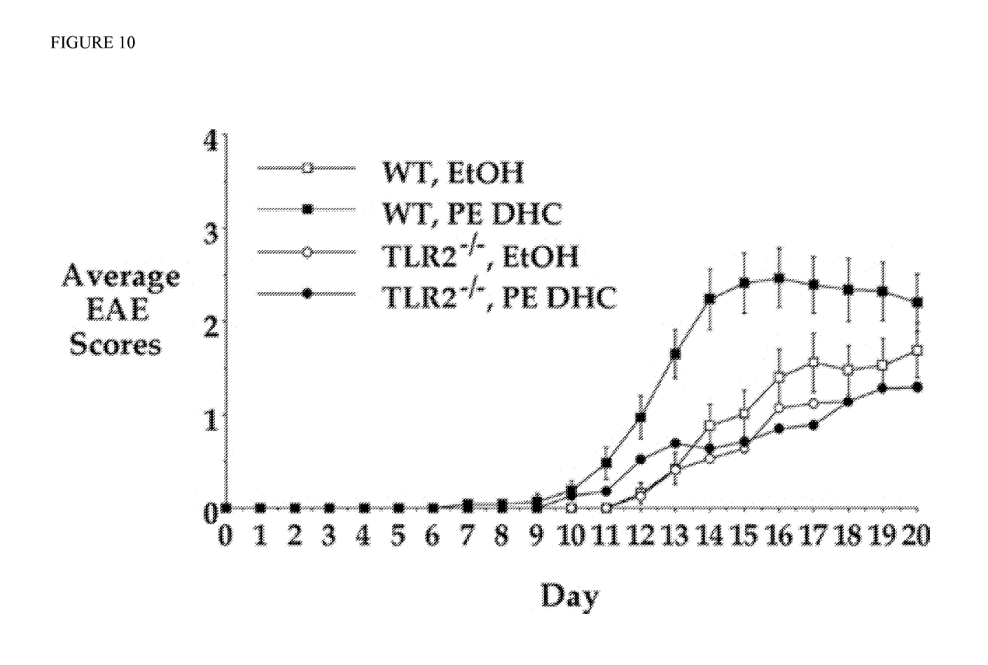

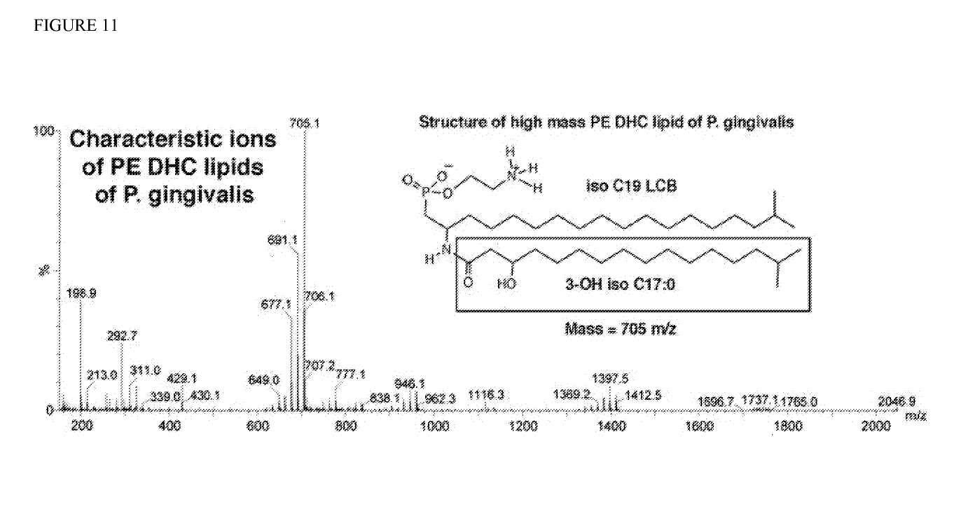

TLR-2 deficient (TLR2^{-/-}) mice were immunized with the standard EAE-inducing MOG protocol and administered a single i.p. injection of either EtOH or PE DHC on Day 0. In contrast to its effect on WT, IL-15^{-/-}, and IL-15Rα^{-/-} mice, PE DHC did not mediate enhancement of CDI or mean daily disease in TLR2^{-/-} mice. As seen in FIG. 10 (a composite of four experiments; n=15 TLR2^{-/-} mice, n=28 WT mice) and in Table 1, PE DHC-treated TLR2^{-/-} mice demonstrated no statistically significant enhancement of EAE CDI, mean daily disease, disease incidence, mean maximal severity or day of onset when compared to EtOH-treated TLR2^{-/-} mice. These results indicated that TLR2 was required for PE DHC to mediate enhancement of EAE.

### Example 12

### PE DHC Enhancement of EAE was not a Result of Contamination with LPS or Lipid A

Since LPS preparations have been shown to influence the development of EAE, it was desirable to establish that PE DHC was not contaminated with Lipid A or LPS. The Bligh and Dyer phospholipid extraction procedure that was used for recovering the P. gingivalis lipids has previously been shown to exclude LPS of P. gingivalis from the organic solvent phase containing the total bacterial lipids. See Nichols “Distribution of 3-hydroxy iC17:0 in subgingival plaque and gingival tissue samples: Relationship to adult periodontitis” Infect. Immun. 62:3753-3760 (1994); Safavi & Nichols “Effect of calcium hydroxide on bacterial lipopolysaccharide” J. Endod. 19:76-78 (1993). Furthermore, P. gingivalis total lipids extracted by this method also did not contain Lipid A species known to be produced by P. gingivalis. The PE DHC lipid fraction was previously characterized using collisional electrospray-MS/MS studies, as described in Nichols et al. “Structures and biological activities of novel phosphatidylethanolamine lipids of Porphyromonas gingivalis,” J. Lipid Res. 47:844-853 (2006). Structural NMR studies were also used. Both studies confirmed the structural characteristics of the lipids and lack of both carbohydrate and protein contaminants in the relevant lipid fraction. In addition, contamination of the fraction with neutral LPS was unlikely because the HPLC separations used a polar column, and the relevant lipid was highly polar and therefore late eluting. All neutral lipid components eluted close to the void volume and were not recovered in the lipid fractions used in these studies. Electrospray-MS evaluation of all the major lipid classes purified by HPLC confirmed that these lipid fractions were not contaminated with Lipid A species of P. gingivalis LPS. Electrospray-MS of the PE DHC lipid fraction of P. gingivalis demonstrated that the characteristic dominant Lipid A negative ions (1195, 1435, 1449, 1690 and 1770 m/z) previously described for P. gingivalis (see Darveau et al. “Porphyromonas gingivalis lipopolysaccharide contains multiple lipid A species that functionally interact with both toll-like receptors 2 and 4” Infect. Immun. 72:5041-5051 (2004) and Reife et al. “Porphyromonas gingivalis lipopolysaccharide lipid A heterogeneity: differential activities of tetra- and penta-acylated lipid A structures on E-selectin expression and TLR4 recognition” Cell Microbiol. 8:857-868 (2006)), were not recovered in this isolate, as illustrated in FIG. 11. Thus, the approach used for preparation of the lipids and the analyses of the lipid fractions ruled out the possibility that the P. gingivalis PE DHC fraction was contaminated with Lipid A or LPS.

### Example 13

### Administration of PE DHC Resulted in Increased Recovery of Bacterial Lipids in the Brains of Mice with EAE

The level of 3-OH isobranched (iso)C17:0 fatty acid was determined in brain specimens of mice with EAE treated with PBS, EtOH or PE DHC. The approach of measuring 3-OH isoC_{17:0} fatty acid in tissues was based on the concept that mammalian tissues, unlike bacteria, have no established biochemical pathway for de-novo synthesis of 3-OH isoC_{17:0} fatty acid. Thus, the recovery of 3-OH isoC_{17:0} fatty acid reflects the presence of bacterially-derived products in the tissue. 3-OH isoC_{17:0} is a constituent fatty acid of all phosphorylated dihydroceramide lipids of P. gingivalis. Nichols et al. (2004)

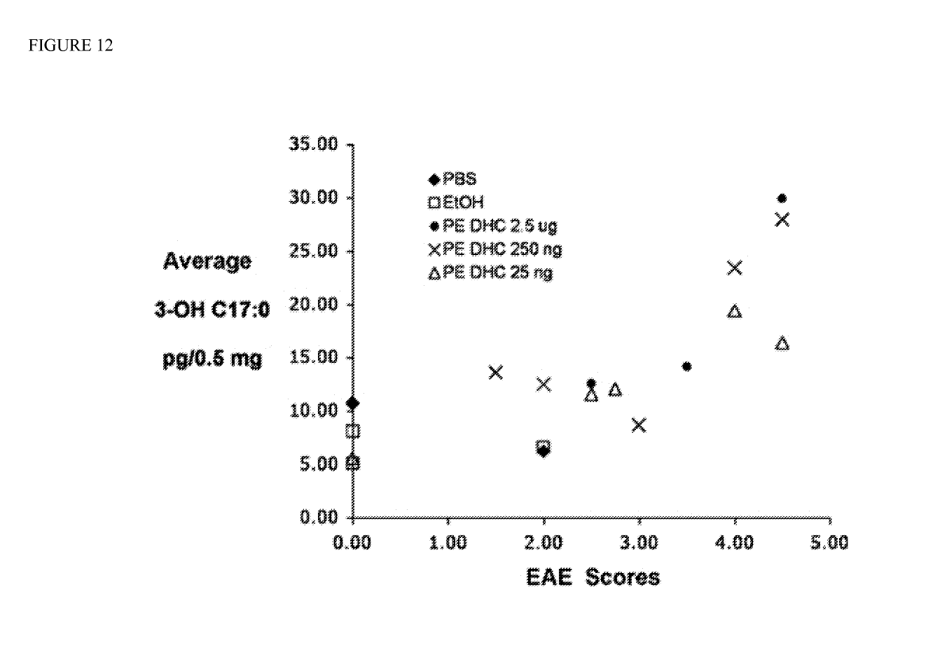

Mice treated with PBS, EtOH or with 25 ng, 250 ng, or 2.5 ug PE DHC were sacrificed after day 20 post-EAE immunization. The brains were removed, extracted for phospholipids, and fatty acid recovery was quantified by selected ion monitoring for fatty acid negative ions. FIG. 12 illustrates the average 3-OH isoC_{17:0} recovery (3 determinations/mouse brain sample) as a function of both the final grade of EAE and the treatment received by each mouse. The data were expressed as picograms of 3-OH isoC_{17:0} per 0.5 mg of total brain lipid extracted. The average S.E.M. for all determinations was +/−2.2 pg/0.5 mg total lipid. As illustrated in FIG. 12, lipids derived from the brains of control (PBS or EtOH-injected) mice showed low levels of recoverable 3-OH isoC_{17:0} fatty acid. These experimental data reflected cumulative exposure of normal mice to complex lipids and/or LPS derived from other commensal bacteria. The experimental results showed higher levels of 3-OH isoC_{17:0} fatty acid in mice that had received PE DHC and had a disease score greater than 3.0, as illustrated in FIG. 12. Linear regression analysis revealed that the correlation between EAE disease score and brain 3-OH isoC_{17:0} fatty acid was directly associated with the dose of PE DHC injected: the strongest correlation (regression coefficient or slope) was seen with the highest dose of PE DHC (2.5 ug, y=11.578+8.690x, R²=0.818), the next strongest with the middle dosage (250 ng, y=2.168+5.014x, R²=0.620), and the weakest correlation with the lowest dose of PE DHC (25 ng, y=3.789+3.062x, R²=0.808).

### Example 14

### PE DHC Activated APCs and Induced IL-6 Secretion In Vitro in a TLR2-Dependent Manner

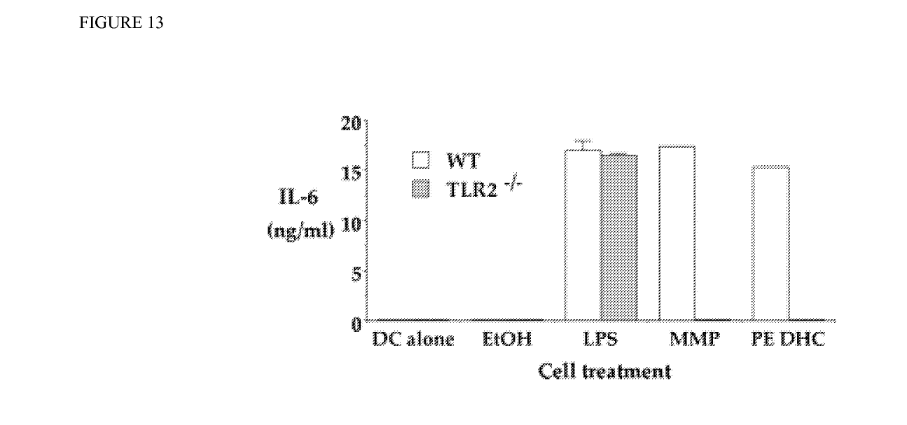

The effects of PE DHC on antigen presenting cell (APC) activation in vitro were examined Dendritic cells (BMDCs) (>85% CD11c+) were derived from the bone marrow of WT or TLR2^{-/-} mice and cultured either alone or with EtOH, LPS, MMP (a TLR2 ligand), or PE DHC. After 18 hrs supernatants were assayed for IL-6. As illustrated in FIG. 13, stimulating WT BMDCs in the presence of LPS or MMP resulted in IL-6 secretion. Culturing TLR2^{-/-} BMDCs in the presence of LPS also resulted in IL-6 secretion, but culturing in the presence of MMP did not. WT BMDCs in the presence of PE DHC demonstrated levels of IL-6 secretion that were almost equivalent to that seen with LPS. However, in contrast to its effects on WT BMDCs, culturing PE DHC with TLR2^{-/-} BMDCs did not result in IL-6 secretion. BMDCs were also assayed for expression of the surface activation markers B7.2 and MHC class II. It was found that PE DHC increased MHC II and B7.2 expression on WT but not TLR2^{-/-} BMDCs. These results indicated that PE DHC can activate DCs and in a TLR2-dependent manner.

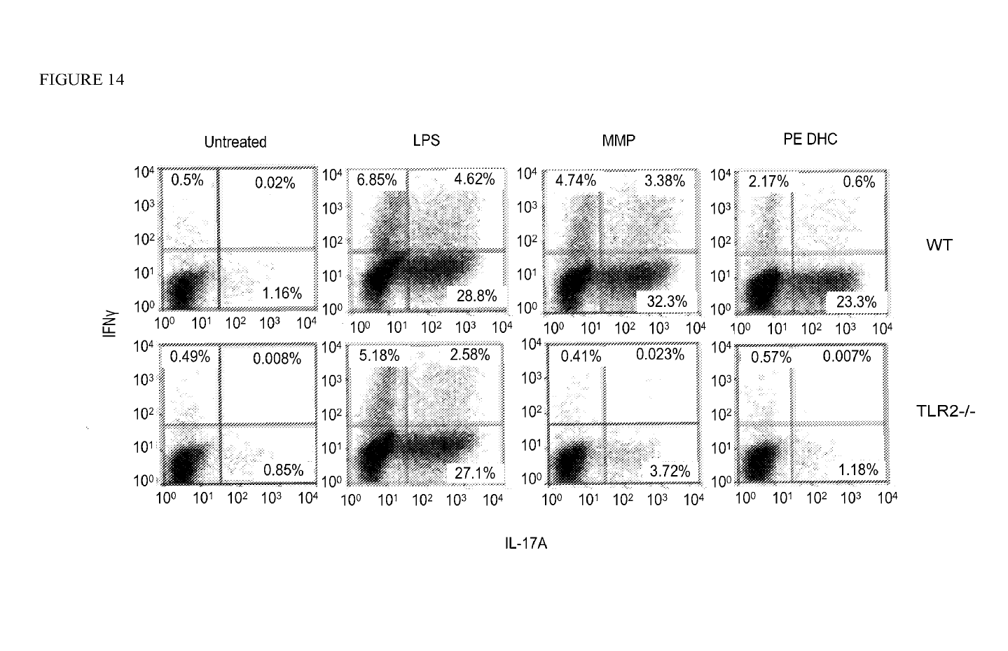

PE DHC's ability to induce IL-6 secretion was characterized by testing its ability to induce Th17 T cell generation from cultures of naïve CD4+ CD25-T cells activated in the presence of APCs (T cell depleted splenocytes; Tds) and TGF-β. See Bettelli et al. “T(H)-17 cells in the circle of immunity and autoimmunity” Nat. Immunol. 2007, 8:345-350. Adding PE DHC resulted in the generation of Th17 T cells in cultures containing WT but not TLR2^{-/-} Tds, as illustrated in FIG. 14. These results further confirmed that PE DHC can induce IL-6 secretion from APCs in a TLR2-dependent manner. When taken together, these results indicated that PE DHC mediates its in vitro and in vivo effects through TLR2-dependent mechanisms.

### Example 15

### PE DHC Decreased the Percentage of CD4+Foxp3+ Spinal Cord Tregs

To characterize mechanisms by which PE DHC may enhance autoimmune disease in vivo, the experimental studies tested whether the PE DHC-mediated enhancement of EAE was associated with alterations in T cell populations at a site of disease. WT mice were immunized with the usual EAE-inducing protocol and treated on Day 0 with EtOH or PE DHC (250 ng i.p.). Within 5 days after onset of EAE, mice were sacrificed and exsanguinated, their spinal cords were removed, and the mononuclear cells were derived from the spinal cords. These cells were analyzed directly for CD4 and Foxp3 expression by flow cytometry or were stimulated with PMA and ionomycin for 4 hours and then, gating on Thy1.2+ cells, analyzed for intra-cellular interferon gamma (IFNγ) and IL-17 by flow cytometry. After sampling mice from three separate experiments, no significant difference were found in the total number of mononuclear cells obtained from the spinal cords of EtOH versus PE DHC-treated mice. In addition, the percentages of spinal cord-derived CD4+ T cells staining for either intra-cellular IFNγ or IL-17 (or cells expressing both cytokines) were not significantly different between EtOH and PE DHC-treated mice. However, the percentage of CD4+ T cells within the total mononuclear cell populations derived from the spinal cords of PE DHC-treated mice was, on average, greater than the percentage in EtOH-treated mice (as illustrated in Table 2). Moreover, while this increase in percentage of CD4+ T cells from PE DHC spinal cords did not reach statistical significance, a statistically significant decrease was observed in the mean percentage of spinal cord CD4+ T cells that were Foxp3+(theoretically representing regulatory T cells; [Tregs]) in the PE DHC-treated mice (p=0.0397) (as illustrated Table 2). The mean percentage of spinal cord cells that were CD4+ was 41% in EtOH-treated mice and, on average, 6.7% of these were Foxp3+. In contrast, the mean percentage of spinal cord cells that were CD4+ T cells was 52% in PE DHC-treated mice and, on average, 4.3% of these were Foxp3+. It has been reported in Korn et al. “Myelin-specific regulatory T cells accumulate in the CNS but fail to control autoimmune inflammation” Nat. Med. 13:423-431 (2007) that the percentage of spinal cord CD4+ Foxp3+ T cells increased as the disease progressed. On average, the PE DHC-treated mice from which spinal cord cells were derived had a slightly longer duration of disease than did the EtOH-treated mice (1.5 days longer; as illustrated Table 2). Based on this observation, it was unlikely that the decrease in the percentage of Foxp3+ in PE-DHC-treated mice was related to differences in disease duration.

### Example 16

### Structures of Lipid 654 and Lipid 430

Bacterial lipids depicted in FIG. 15A-C (“Lipid 654” and “Lipid 430”), with their respective precursor masses and characteristic ion fragments, and an NMR profile for the Lipid 654 consistent with that of the bacterial lipid previously termed “Flavolipin,” is produced by many bacteria found commonly in the oral cavity and gastrointestinal tract of normal individuals. This is in contrast to prior reports (described below) that Flavolipin is uniquely produced by the rare Flavobacterium species, which include Flavobacterium meningosepticum, an opportunistic pathogen that causes disease in immunocompromised individuals and infants, but not in healthy adults. See Kawasaki et al., J Endotoxin Res. 2003; 9(5):301; Gomi et al., J Immunol. 2002 Mar. 15; 168(6):2939-43; Kawai et al., Eur J Biochem. 1988 Jan. 15; 171(1-2):73-80; and Kawai et al., Infect Immun. 1989 July; 57(7):2086-91.

The Bacteroidetes Phylum, represented by many genera of organisms recovered in both the oral cavity and gastrointestinal tract, are the organisms known to produce the Lipid 654 and also likely produce Lipid 430. However, the organisms reported to produce Flavolipin by Kawai et al., Eur. J. Biochem., 171:73-80, 1988, included only Flavobacterium meningosepticum, F. indologenes, Achromobacter xylosoxidans, Pseudomonas jluorescens, P. aeruginosa, P. cepuciu, and P. stutzeri. Flavobacteria are typically recovered in low amounts in the oral cavity or the gastrointestinal tract and these organisms are not associated with either oral or gastrointestinal disease. Oral Bacteroidetes shown to produce Lipid 654 include Porphyromonas gingivalis, Prevotella intermedia, Tannerella forsythia, Capnocytophaga ochracea, C. gingivalis and C. sputigena. Selected Bacteroidetes genera of the gastrointestinal tract have been evaluated for Lipid 654. Of those intestinal isolates tested, Prevotella copri, Parabacteroides merdea, Bacteroides fragilis, and B. vulgatis also produce the Lipid 654. Many of these organisms are commensals either in the oral cavity or gastrointestinal tract. However, some of these organisms are considered to be opportunistic pathogens.

Negative ion fragments for Lipid 654, determined by MS/MS, are depicted in FIG. 15A. Two other lipid species exist with negative ion masses of 640 and 626 but are otherwise similar in structure to the Lipid 654. Although these three lipid species cannot be separated by presently available chromatographic approaches, they are known to have similar structures and all three are presumed to possess the biological activities described below.

The structure of the dominant Lipid 654 species (m/z 654) is derived from the mass spectra and NMR spectra of the purified Lipid 654 of Porphyromonas gingivalis (P. gingivalis). The Lipid 654 class was prepared by extracting total lipids of P. gingivalis using the phospholipid extraction procedure of Bligh and Dyer, Can. J. Biochem. Physiol. 1959 37:911-917 and the lipids were fractionated by semipreparative HPLC as described in Nichols et al., J Lipid Res. 2004 45(12):2317-30. The fractions demonstrating TLR2 activity were evaluated by single stage mass spectral (MS) and MS/MS using an ABSciex QTrap 4000 instrument (Framington, Mass.). The parent negative ion mass of Lipid 654 (dominant lipid species) was determined by single stage MS analysis and the negative ion fragments were identified using MS/MS analysis and are depicted in the chemical structure in FIG. 15A. When using the QTrap instrument in the positive ion mode, the parent ion mass and fragment ions were detected as shown in FIG. 15B. Sodium methoxide treatment yielded primarily isobranched C_{15:0} as measured using GC-MS, thus confirming the esterified fatty acid shown in FIGS. 15A and 15B. Furthermore, hydrolysis of the Lipid 654 class with concentrated HCl and analysis of amino acid derivatives by chiral column GC-MS demonstrated that Lipid 654 contains L-serine. The epimeric form of the beta hydroxy carbon on the seventeen carbon fatty acid has yet to be determined. The Lipid 430 class, produced by P. gingivalis, has not been reported previously. This lipid represents three lipid species with negative ion masses of m/z 430, 416 and 402 (see FIG. 15C). Lipid 430 also activates HEK cells through TLR2. The primary difference between the Lipid 654 class and the Lipid 430 class is that the Lipid 430 class is soluble in neutral or basic aqueous solutions but is not soluble in acidic aqueous solutions. In contrast, the Lipid 654 class is soluble in organic solvents.

### Example 17

### Lipid 654 Functions as a Ligand for TLR2

Lipid 654 mediates significant effects on the innate immune system as evidenced both in vivo in mice and in vitro with human cells. However, in contrast to prior reports of Flavolipin, Lipid 654 functions as a ligand for TLR2 and not as a ligand for TLR4.

In vitro effects, human cells: HEK293 cells (human embryonic kidney cells) transfected with the human TLR2 and SEAP (secreted embryonic alkaline phosphatase) genes, were used to assay the function of Lipid 654 in vitro. The HEK cells, which naturally express variable levels of TLRs 1, 3, 5, 6, 7 and 9, were also transfected with the gene for CD14. CD14 is a co-receptor that enhances TLR2 responses. Additionally, TLR2 responses are most often mediated via another co-receptor which, in most cases, is either TLR1 or TLR6. The SEAP reporter gene is under the control of the IFN-β minimal promoter fused to five NF-κB and AP-1-binding sites. Stimulation with a TLR2 ligand activates NF-κB and AP-1 which induce the production of SEAP which is then quantitated as a colorimetric change in the presence of a detection medium.

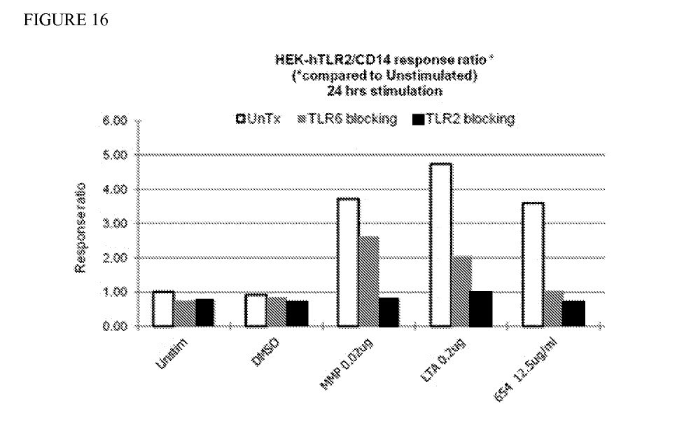

Lipid 654, solubilized in 50% DMSO in water, and two documented TLR2 agonists, MMP and lipotechoic acid (LTA), were incubated for 24 hours with the HEK cells in the presence or absence of antibodies to TLR2 and TLR6. As seen in FIG. 16, MMP and LTA demonstrated NF-κB-activation and this activation was inhibited by antibodies both to TLR2 and TLR6. Significantly, Lipid 654 also demonstrated NF-κB-activation and this activation was also inhibited by anti-TLR2 and anti-TLR6 antibodies. Thus, Lipid 654 is a TLR2 agonist and the co-receptor for this TLR2-mediated activation is TLR6.

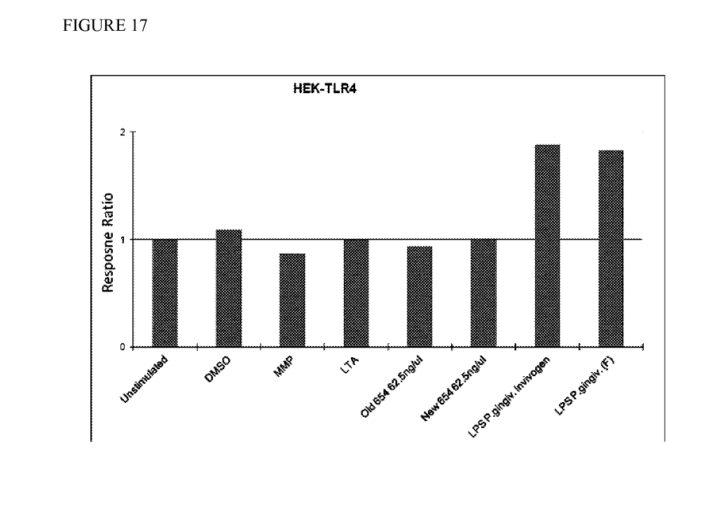

Prior reports stated that Flavolipin could induce innate immune activation by acting as an agonist for TLR4 (Gomi et al., J Immunol. 2002 Mar. 15; 168(6):2939-43). To demonstrate that, in contrast to previous reports, Lipid 654 was not a ligand for TLR4, HEK293 cells transfected with the human TLR4 and CD14 genes, but not expressing TLR2, were utilized. As shown in FIG. 17, the known TLR4 agonist lipopoysaccharide (LPS) (in this case derived from P. gingivalis) demonstrated the ability to stimulate the TLR4-expressing cell line. In contrast, the TLR2 agonists, MMP and LTA, showed no activity. Two different preparations of Lipid 654 (“old” and “new” in FIG. 17) showed no ability to activate the TLR4-expressing cell line. This indicates that, in contrast to Flavolipin, Lipid 654 can activate via TLR2 but is unable to function as a TLR4 ligand.

Lipid 430 and TLR2

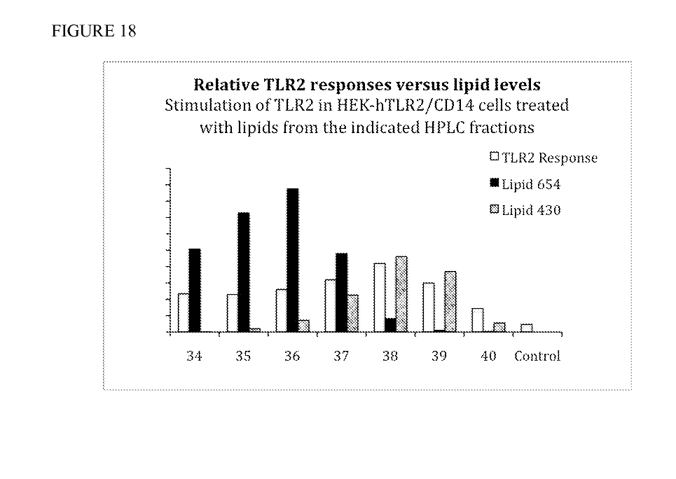

The recovery of Lipid 654 and Lipid 430 in specific HPLC fractions was compared with the relative capacity of these HPLC fractions to activate TLR2 in HEK-hTLR2/CD14 cells. Total lipids of P. gingivalis were fractionated by semi-preparative HPLC and fractions 34 through 40 were shown to promote TLR2 activation in HEK cells (see FIG. 18). These fractions were then evaluated by ESI-MS for the Lipid 654 and Lipid 430 preparations. FIG. 18 shows that both the Lipid 654 and Lipid 430 lipids show the capacity to activate human TLR2 in HEK cells.

To confirm these findings using in vivo approaches, mice were injected with Lipid 654 (according to the procedure described below) and the effect on serum levels of the chemokine, CCL2 (also known as MCP-1), was analyzed. This chemokine has been demonstrated to be critical in the development of experimental autoimmune encephalomyelitis (EAE), the murine model of Multiple Sclerosis (MS), and is also believed to be critical in the pathogenesis of (human) MS. CCL2 plays a major role in mediating the migration of inflammatory macrophages into tissue sites of inflammation such as the central nervous system in MS. It has been previously documented that administration of TLR agonists to mice can result in enhanced serum levels of CCL2.

Lipid 654 was injected intraperitoneally (i.p.) into mice and three to four hours later serum was drawn and analyzed for levels of CCL2 via ELISA. Both female wild type (WT) C57BL/6 mice and female TLR2-deficient (TLR2−/−) mice were injected i.p. with either DMSO (vehicle control; VC) or Lipid 654. Three to four hours later, serum was drawn from these mice and analyzed for levels of CCL2. As described below in Example 23, Lipid 654 induced a significant increase in serum levels of CCL2 in WT mice, but failed to do so when injected into TLR2−/− mice. These results confirmed the in vitro findings described above and demonstrated that Lipid 654 has the potential to be pro-inflammatory in vivo and requires TLR2 to mediate these effects.

### Example 18

Lipid 654 is produced by bacteria but is not produced by mammals and, as a result, Lipid 654 can be identified and quantified in human tissue. Lipid 654 was recovered in the serum, gingiva, and brain of normal individuals. Lipid 654 includes isobranched aliphatic chains within its constitutive fatty acids. Furthermore, a minor percentage of the C_{15:0} fatty acid is recovered as anteisobranched fatty acid. The fatty acid in Lipid 430 is also isobranched. These structures have not been described in mammalian lipids. To confirm the bacterial origin of Lipid 654, multiple reaction monitoring mass spectrometry (MRM-mass spec) was used. MRM-mass spec allows for highly accurate identification and quantification of individual molecules within complex mixtures of molecules. As sources of non-bacterially-exposed mammalian tissues, surgically excised human 3^{rd} molars (tissue not directly exposed to the oral cavity) were analyzed, in addition to brain samples from germ-free mice, for the presence of Lipid 654. Neither the human 3^{rd} molars nor the murine brain samples from germ-free mice demonstrated detectable levels of Lipid 654. Thus, mammalian tissues do not produce Lipid 654.

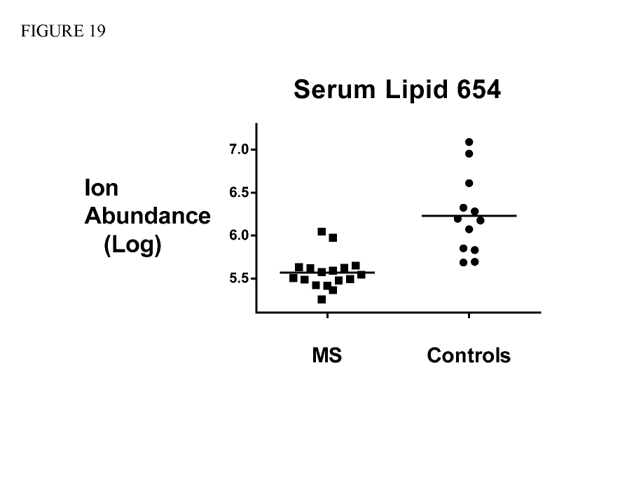

In contrast, MRM-mass spec analysis of human serum samples (see FIG. 19), samples of frozen human brain specimens, and samples of diseased human gingival tissue revealed significant levels of Lipid 654. Thus, bacterial-derived Lipid 654 normally gains access to the human vasculature (human serum), human brain, and also to sites of inflamed human tissue.

### Example 19

Variations in the amounts of Lipid 654 in human serum provide a new biomarker of multiple sclerosis (MS) and other inflammatory diseases.

To assess the role of Lipid 654 in the underlying mechanisms involved in the pathogenesis of MS, serum samples from control and MS patients were analyzed for levels of Lipid 654. Twelve healthy individuals and 17 patients with MS donated blood samples and the serum lipids were analyzed for the presence of Lipid 654 using MRM-mass spec.

The ages of the healthy control and MS patients ranged from 19 to 63 years old. Both men and women were equally represented. The MS group included patients recently diagnosed, those with more chronic disease, and patients who were either untreated or on various therapeutic regimens. Blood was obtained by venipuncture and, after clotting, the tubes were spun to yield serum and frozen at −80° C. Subsequently, the frozen serum samples were thawed, 0.5 ml of each was extracted, and 500 ug of serum lipid was derived from the chloroform:methanol fraction.

Before mass spectral analysis, a standard amount of ¹³C lipid (with a peak mass of 660) internal standard was added to each sample. The internal standard lipid was prepared by culturing P. gingivalis in broth medium containing 0.5 g/l of ¹³C acetate. After four days of culture, the bacteria were pelleted by centrifugation and lyophilized. The bacterial pellet was then extracted for total lipids and the fraction containing lipids consistent with the Lipid 654 class was identified by semipreparative HPLC. The ¹³C-substituted Lipid 654 class was determined to have peak mass of m/z 660 but contained only 1-2% unlabeled authentic Lipid 654. Lipid 654 was determined to have a base peak mass of m/z 660 but contained only 1-2% unlabeled authentic 654 lipid. This lipid fraction was then used as an internal standard for supplementing human serum samples. The QTrap instrument was used for MRM-mass spec. Samples were infused with a Shimadzu HPLC pump interfaced with an autosampler. Each sample was run over a short normal phase HPLC column (Ascentis® Si, 10 cm×2.1 mm, 5 um, Supelco Analytical) at 0.1 m/min using a solvent system consisting of hexane:isopropanol:water (6:8:0.75, v/v/v). Three transitions were monitored for Lipid 654 against two dominant transitions for the internal standard lipid and each characteristic transition was integrated electronically. The peak areas for each transition were normalized against the internal standard.

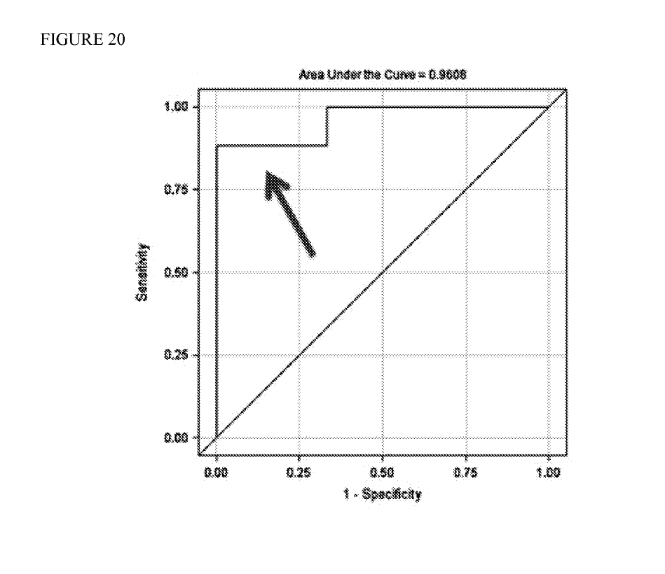

MRM-mass spec analysis of the serum lipids revealed significant differences in levels of Lipid 654 between control and MS patients. As shown in FIG. 19, the mean level of Lipid 654 in the serum samples of healthy individuals was significantly greater than the levels in the serum of MS patients (p=0.0005271). These results represent the first significant identifiable serum marker capable of distinguishing MS patients from healthy individuals. The suitability of Lipid 654 for diagnostic use was confirmed by ROC curve analysis, which indicates that Lipid 654 can differentiate between MS patients and non-MS patients (see FIG. 20). The ROC curve shows a sensitivity of 94% and specificity of 92% with an ion abundance of 118,585. As such, measuring serum levels of Lipid 654 allows for a new clinical approach to the diagnosis of MS and represents the first blood test for such a diagnosis. In addition, serum levels of Lipid 654 represent a new biomarker for identifying disease-activity in MS. Chronic administration of drugs used to treat MS often results in a high frequency of adverse side-effects. The potential for serum levels of Lipid 654 to predict or identify, in the earliest stages, disease activity in MS would allow for the first clinical tool for individualizing therapeutic intervention in MS. Such a predictive blood test would make possible intermittent, rather than chronic, administration of therapy. In sum, the approach described herein provides a new clinical approach to MS and potentially to other autoimmune and inflammatory diseases as well.

### Example 20

### Lipid 654 in Human Serum Inhibits Experimental Allergic Encephalomyelitis (EAE)

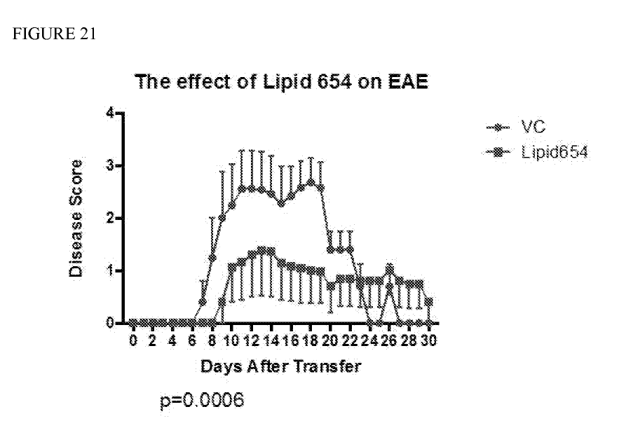

Female SJL mice were injected intra-peritoneally with 30×10⁶ 4-day in-vitro-activated proteolipid protein (PLP)-stimulated lymph node lymphocytes and on the same day injected intravenously with either phosphate buffered saline (PBS) (vehicle control=“VC”) or 2 ng of Lipid 654. Mice were followed for 30 days for development of EAE and scored as 1=tail paralysis; 2=abnormal gait; 3=paralyzed hind legs; 4=paralyzed front and hind legs; 5=death. The results in FIG. 21 depict the daily mean for 5 mice injected with the VC and 5 mice injected with Lipid 654. As shown in FIG. 21, Lipid 654 significantly inhibited the course of EAE compared to vehicle control. Thus, MS patients can be treated directly with Lipid 654 or with commensal bacteria that produce increased amounts of Lipid 654. This result is consistent with the findings herein that Lipid 654 in the serum keeps the systemic immune system under control. Therefore, lower amounts of Lipid 654 in the serum of MS patients show that the deficit in Lipid 654 has a role in the cause of MS.

### Example 21

### Enzymatic Hydrolysis of Lipid 654 by Phospholipase A2

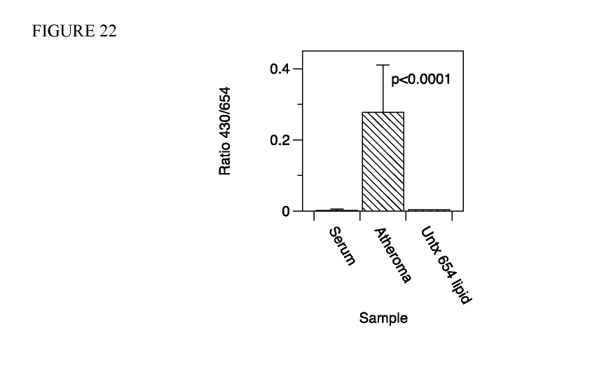

Of the many potential explanations for the reduced levels of Lipid 654 in serum of MS patients, the possibility exists that enzymatic hydrolysis of Lipid 654 may occur to a greater extent in the blood of MS patients when compared with otherwise healthy subjects. Not to be bound by theory, enzymatic breakdown of Lipid 654 is most likely caused by esterase hydrolysis of the ester linkage resulting in the generation of Lipid 430. Lipid 430 is a strong TLR2 agonist but is also water soluble. The levels of Lipid 430 relative to Lipid 654 therefore provide a measure of enzymatic hydrolysis in human samples. Supporting evidence comes from an examination of Lipid 654 levels relative to Lipid 430 in sera samples versus samples of arteries where a chronic inflammatory reaction occurs in association with atherosclerotic plaque development. The levels of Lipid 430 and Lipid 654 were quantified in serum samples from healthy subjects and the ratio of Lipid 430 to Lipid 654 was calculated for these samples (n=12). Lipid extracts from human carotid atherosclerotic plaques (n=10 samples of excised carotid artery samples recovered during endarterectomy procedures) were also evaluated at the same time. This comparison revealed the results shown in FIG. 22.

As shown in FIG. 22, results showed a significant difference between serum and carotid artery samples (p<0.0001; Mann-Whitney test). The mean Lipid 430/Lipid 654 ratio increased in carotid artery walls by greater than three orders of magnitude over serum levels. Mass action dependent diffusion cannot account for such a relative increase in Lipid 430. Not to be bound by theory, the increase in Lipid 430/Lipid 654 ratio likely results from accumulation of Lipid 654 in artery tissues and the accompanying chronic inflammatory response leads to hydrolytic breakdown of Lipid 654 to Lipid 430 through the expression of phospholipase A2.

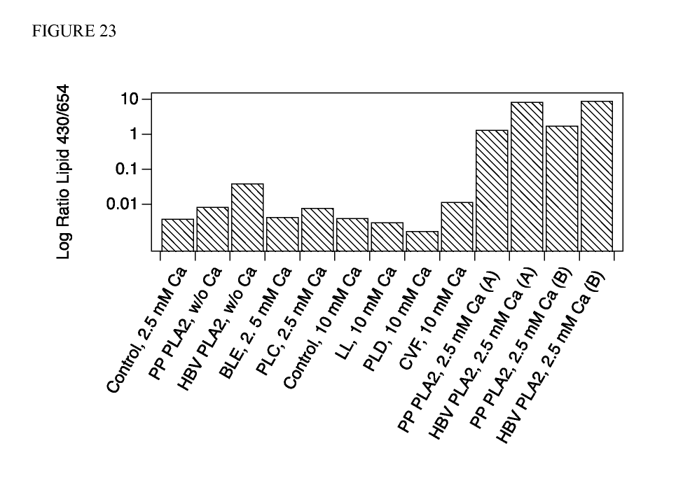

A survey of common lipid hydrolyzing enzymes revealed that Lipid 654 is hydrolyzed by phospholipase A2 (PLA2) enzymes, either mammalian PLA2 (porcine pancreas) or honey bee venom PLA2, as shown in FIG. 23. Lipid 654 was prepared, largely free of Lipid 430 as shown in the Controls. Aliquots of the enriched 654 (10 μg each) were dispensed into conical vials and the residual solvent was dried. Tris-buffered (10 mM) saline containing either no calcium/0.01% sodium EDTA, 2.5 mM calcium, or 10 mM calcium was added to specific vials (1.0 ml) and the solution was sonicated for 30 seconds. Specific vials were then supplemented with either porcine pancreatic phospholipase A2 (PP PLA2), honey bee venom PLA2, bovine liver nonspecific esterase (BLE), phospholipase C (PLC), lipoprotein lipase (LL), phospholipase D (PLD), or cobra venom factor (CVF). PP PLA2 or HBV PLA2 was added at a concentration of 96 or 120 U/ml, respectively. All other lipase preparations were used at this or a higher concentration. Controls were dissolved in the indicated Tris buffer but were not supplemented with enzyme. Each vial was stirred for 3 days after which the hydrolyzate was acidified with 100 ul of glacial acetic acid and extracted three times with chloroform. The pooled extracts were dried, reconstituted in HPLC solvent, and evaluated for recovery of Lipid 654 and Lipid 430 using MRM-MS as previously described. Two different isolates of Lipid 654, indicated as (A) and (B), were compared for hydrolysis by either porcine pancreatic or honey bee venom PLA2. The ratios of Lipid 654/430 are depicted in FIG. 23 as the Log (10) values.

Phospholipase A2 enzymes are recovered in all tissues and levels of this enzyme are increased in association with virtually all chronic inflammatory diseases. In fact, phospholipase A2 inhibitors are being used to treat atherosclerosis, multiple sclerosis, and a variety of other conditions. Phospholipase A2 hydrolyzes glycerol phospholipids so that the ester linked fatty acid in the #2 carbon position of glycerol is released, but only for a glycerol ester linkage in the L enantiomeric configuration. Phospholipase A2 will not hydrolyze fatty acid linked in the D enantiomeric form of glycerol. Lipid 654 is not a glycerol phospholipid and yet, phospholipase A2, but no other common lipase enzymes, will hydrolyze Lipid 654 to Lipid 430. Lipid 430 released with PLA2 treatment of Lipid 654 is a strong TLR2 agonist in mouse bone cells and is at least ten fold more potent than the Lipid 654 preparation in engaging TLR2.

Additionally, PLA2 enzymes do not completely hydrolyze Lipid 654 preparations from P. gingivalis. Repeated treatment of Lipid 654 preparations with PLA2 did not reveal complete hydrolysis of the Lipid 654. Approximately half of the Lipid 654 is not hydrolyzed by PLA2, suggesting that Lipid 654 is composed of two isomers that differ in enzyme susceptibility. Ion mobility mass spectrometry to show that Lipid 654 is composed of two isomeric forms with identical structural characteristics (by MS/MS analysis). The reduced levels of Lipid 654 in serum of Multiple Sclerosis subjects could be related to a higher phospholipase A2 activity in serum of these subjects. The Lipid 654 which is not hydrolyzed by PLA2 is a weak TLR2 agonist.

### Example 22

### Lipid 654 is a Microbiome-Associated Biomarker for Multiple Sclerosis

Materials and Methods

Patient samples: Healthy controls and patients with MS were recruited. Patients with MS were recruited both from the MS clinic at UCHC as well as from other physicians in the state of Connecticut. Samples were only drawn from patients who had not eaten for at least 2 hours. Blood samples in clotting tubes remained at room temperature for exactly 1 hour. After centrifugation to separate the serum, the serum was pipetted into glass tubes using glass pipettes and frozen immediately at −80° C. Only glass pipettes were used in all subsequent handling of the serum samples to avoid adsorption of lipids to plastic.

Derivation of serum lipid samples: All blood samples were stored frozen until processing. For lipid extraction, the serum samples were thawed and lipids were extracted from 0.5 mL of each sample in organic solvent. After drying the organic solvent extracts under nitrogen, the lipid extracts were reconstituted in hexane: isopropanol:water (high-performance liquid chromatography (HPLC) solvent, 6:8:0.75, v/v/v) and vortexed. A small amount of each sample was dried and weighed, and a defined amount of each sample (500 μg) was transferred to a clean glass vial for either further processing or for MRM-mass spectrometry analysis.

Internal standard: The serine lipid internal standard was prepared by culturing Porphyromonas gingivalis in the brain—heart infusion broth supplemented with ¹³C(1)-sodium acetate (0.5 g 1⁻¹). Bacteria were harvested by centrifugation and total lipids were extracted and fractionated as described previously. The lipid fraction with an ion mass of m/z 660.7 co-eluted with Lipid 654 using the HPLC chromatographic conditions described above. Identity of the internal standard was further confirmed by tandem mass spectrometry of its fragment ions. The background level of m/z 653.5 lipid in this internal standard lipid preparation was only 1.5% of the abundance of the m/z 660.7 species, and the Lipid 654 recovered in serum samples was corrected for carryover of m/z 654.5 in the internal standard. This ¹³C-labeled lipid fraction was used as the internal standard for quantifying Lipid 654 in serum samples. A calibration curve was generated using serially diluted lipid 660.7 standard added to vials containing serum lipids. The detection limit of lipid 660.7 was determined to be 50 fmol ml⁻¹ of serum sample. The upper limit of dynamic range for quantitation was 10 nmol ml⁻¹ of serum matrix. Linearity of quantitation was observed with the regression coefficient of R²>0.998.

Mass spectrometry: The serum lipid samples were injected using a Shimadzu (10ADVP) HPLC system interfaced with a QTrap 4000 mass spectrometer (AB Sciex, Framingham, Mass.). Samples were introduced with a Shimadzu SIL-10A automatic sampler (Shimadzu North America, Columbia, Md.) and were eluted over a normal phase silica gel column (2.1 mm×10 cm, 5 μm; Ascentis; Supelco; Sigma-Aldrich, St Louis, Mo.) using isocratic separation with HPLC solvent as described above and the column temperature maintained at 40° C. A lipid profiling analysis was performed under single quadrupole mass spectrometry mode to screen the lipid constituents in the serum samples, and the chromatographic parameters were set accordingly. The flow rate of HPLC separation was 0.15 mL min⁻¹, for a period of 21 minutes, after which the solvent flow was increased to 0.25 mL min⁻¹ for 9 min and a 10-port switching valve diverted residual lipid products to waste. The flow rate was then returned to 0.15 μL, min⁻¹ to stabilize for the next sample injection.

When both healthy control individual and MS patient samples were analyzed, the injection of MS and control serum lipid samples were alternated during MRM analyses. For every 4-5 samples analyzed, blank samples and internal standard samples were introduced to verify minimal carryover of serum Lipid 654 between samples. The optimal ion transitions, unique to Lipid 654, were chosen from previously acquired tandem mass spectra using product ion scan mode. The MRM collision energy and declustering energy were optimized for three selected product ion transitions using ramp scanning of the potentials while directly infusing the highly enriched Lipid 654. The optimal collision energy and declustering energy potentials for Lipid 654 and internal standard lipids were −52 and −90 V, respectively. Both entrance and collision cell exit potentials were set to −10 V. Lipid ion transition peaks were integrated using the Analyst software feature, and the percentage abundance of each lipid class was calculated from the integrated ion transition peaks.

Statistics: Wilcoxon's rank-sum test was used to determine significant differences between sample categories.

Lipid 654 is Found in Serum from Normal Individuals

Lipid 654 is produced by bacteria commonly found in the human oral cavity and GI tract and demonstrates human and mouse TLR2 agonistic function. To assess whether Lipid 654 gains access to the systemic circulation, serum samples were obtained from 12 healthy individuals. These healthy individuals included eight female subjects and four male subjects ranging in age between 33 and 75 years

Total lipids were derived from the serum samples using a phospholipid extraction procedure and MRM-mass spectrometry was used to detect the presence of Lipid 654. MRM-mass spectrometry was chosen as the method of Lipid 654 quantitation because this mode of mass spectrometry offers maximal sensitivity, selectivity and dynamic range and is a critical approach in targeted lipidomics. Three major transition ions of Lipid 654 (as determined by tandem mass spectrometry: Transition 1, m/z 653.5-381.4; Transition 2: m/z 653.5-349.3; and Transition 3: m/z 653.5-131.1) were then used to quantify the recovery of Lipid 654 in human serum samples.

Surprisingly, Lipid 654 was detected in all 12 healthy control-derived lipid serum samples analyzed. Lipid 654 was verified by demonstration of all three characteristic MRM transitions that appear at the expected retention time for this lipid. These results represent the first demonstration that Lipid 654, derived from commensal bacteria inhabiting GI or oral sites, routinely gains access to the systemic circulation in healthy humans.

Lipid 654 is Found in Significantly Lower Levels in Serum from MS Patients Versus Healthy Individuals

In MS, as with most autoimmune diseases, the pathogenesis is believed to involve both genetic and environmental factors. Although no infectious agent has yet been definitively shown to be involved in the pathogenesis of MS, there has been considerable recent interest in the potential role of commensal bacteria in MS and other autoimmune diseases. On the basis of this potential involvement of commensal bacteria in MS, it was determined whether Lipid 654 could be an ‘environmental’ factor mediating the effects of mucosal commensal bacteria on the pathogenesis of MS.

To address this question, serum samples were obtained from 17 patients with MS. These MS patients included 12 female subjects and five male subjects ranging in age from 18 to 84 years. These patients primarily carried a diagnosis of relapsing-remitting MS (two had a diagnosis of secondary progressive MS and one a diagnosis of progressive relapsing MS) with lengths of disease duration ranging from 3 months to 40 years. This cohort included patients being treated with various drug regimens (e.g., prednisone, interferon β-1a, fingolimod, or glatiramer acetate), patients not treated at the time of the blood sampling, and patients never treated for MS. Total lipids were derived from each of the 12 healthy control and 17 MS patient serum samples and resuspended 0.5 mg of total lipid from each sample in an equal volume of solvent. MRM-mass spectrometry was used to compare the levels of serum Lipid 654 in these samples. These MRM-mass spectrometry analyses were run on three separate occasions (‘Run 1, 2 or 3’).

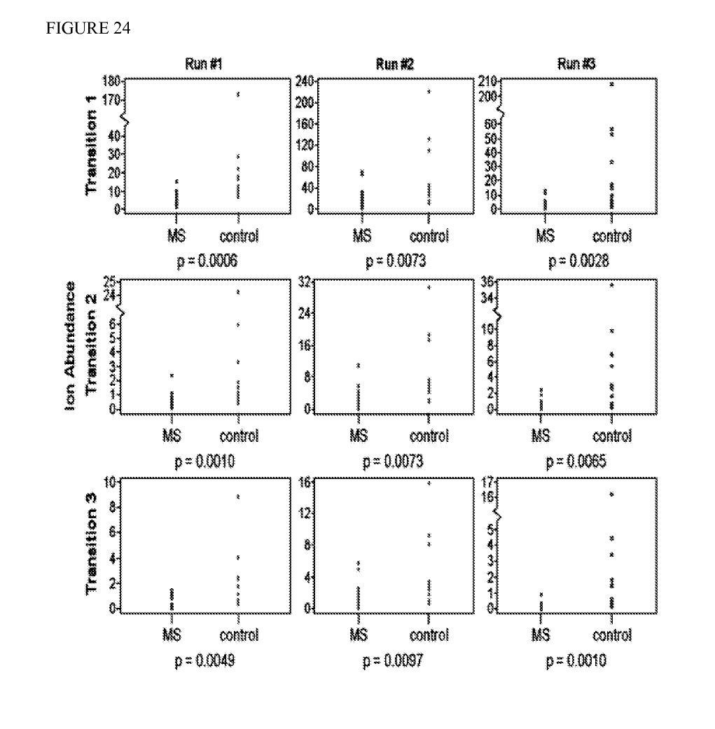

FIG. 24 depicts the three separate MRM-mass spectrometry analyses (vertical columns indicating ‘Run 1, 2 or 3’) comparing serum levels of Lipid 654 in the 17 MS versus 12 healthy control serum samples. Lipid 654 was identified and quantified in each of these three analyses using three major Lipid 654 daughter ions (Transitions 1-3). As shown in FIG. 24, levels of Lipid 654 were significantly and consistently lower in MS serum samples than in serum samples from healthy individuals. This was true for each of the three transitions and in each of the three runs. In FIG. 24, the values for all of the 17 MS patients are represented but are essentially clustered together at the lower levels of ion abundance. In these analyses, the statistical differences between Lipid 654 levels in MS versus healthy control samples ranged from a P-value of 0.0097 to a P-value of 0.0006 (FIG. 24).

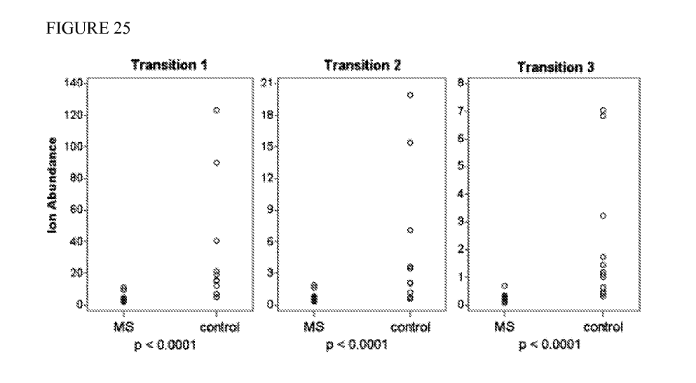

To correct for potential variations arising from slight chromatographic and mass spectrometric alterations occurring during the analysis, an internal standard was added to each sample for Run no. 3. This internal standard permitted quantification of Lipid 654 by correcting, for example, for diminished instrument sensitivity with increasing sample number or variations in lipid infusion by the automatic sampler. As seen in FIG. 25, adjusting for the efficiency of Lipid 654 detection using the internal standard yielded essentially identical results as those depicted in FIG. 24, that is, a very significant difference in serum levels of Lipid 654 between MS patients and healthy controls was found again. In FIG. 25, the values for all of the 17 MS patients are clustered together at the lower levels of ion abundance.

The age and gender distribution of MS and healthy populations in this study were generally similar. In the population of MS patients, there was no significant correlation of serum Lipid 654 levels with the subtype of MS, duration of disease, gender, age or treatment modality. Receiver operating characteristic curves were generated using the results depicted in FIG. 25. The internal standard adjusted data shown in FIG. 25 revealed that at an appropriate ion abundance cutoff value for each transition, the specificity of low serum Lipid 654 levels for MS patients ranged from 83 to 99% and the sensitivity associated with these specificity values ranged from 82 to 94%. These results show for the first time that a unique bacterially derived serine containing lipodipeptide is differentially recovered in the serum of MS patients compared with healthy individuals and that serum levels of Lipid 654 may prove to be a clinically relevant serum biomarker for MS.

Lipid 654 is Found in Significantly Lower Levels in Serum from MS Patients Compared with Alzheimer's Patients

It was then determined whether the levels of serum Lipid 654 in MS patients would also be lower than those in patients with other neurological diseases. For this study, frozen serum samples were obtained from MS patients and from patients with Alzheimer's disease. The specimens were handled utilizing universal and standard methods for obtaining serum samples. The samples included those derived as post-mortem specimens.

From the UCLA serum bank, the MS patient samples included those from 13 male subjects, all carrying a diagnosis of primary progressive MS. These MS patients had an age range of 45-81 years. No treatment information was available for these MS patients. One of the 13 MS patient samples was a post-mortem specimen. The Alzheimer's patient samples included those from eight female subjects and seven male subjects with an age range of 59-94 years. All of the Alzheimer's samples were post-mortem samples.

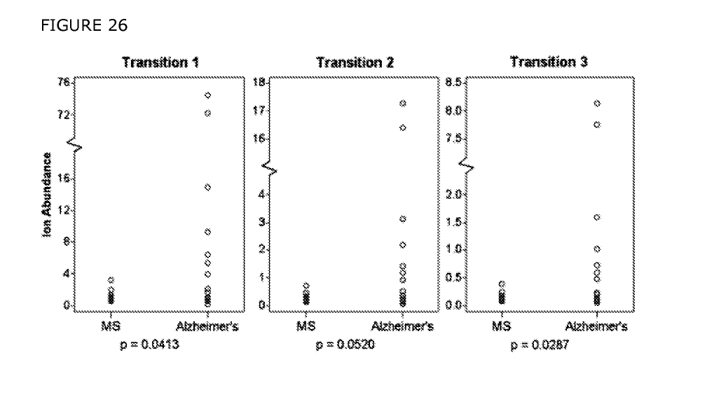

Total lipids were derived from these serum samples and analyzed for levels of Lipid 654 using MRM-mass spectrometry as described above. Levels of expression of Lipid 654 were quantified using Transitions 1, 2 and 3 (see FIGS. 24 and 25). The levels of Lipid 654 in these MS serum samples were significantly lower than those of the Alzheimer's patients (FIG. 26). Two of the three transitions demonstrated statistically significant differences between the MS and Alzheimer's samples, whereas the other transition yielded a P-value of 0.052. As shown in FIG. 26, two Alzheimer's samples demonstrated extremely high serum expression of Lipid 654. These values are consistent with overall findings that MS serum samples demonstrate low Lipid 654 expression. However, dropping these two high expressors from the data resulted in the P-values becoming slightly nonsignificant, but the interpretation of the data remains unchanged.

### Example 23

### Lipid 654 and Lipid 430 are Human and Mouse Toll-Like Receptor 2 Ligands

Materials and Methods

Reagents: BBL Biosate peptone, Trypticase peptone, yeast extract, and brain heart infusion (BHI) broth were obtained from Fisher Scientific. Neutralizing human and mouse anti-TLR2 antibodies, anti-TLR6 antibodies, and anti-TLR1 antibodies were obtained from InvivoGen (San Diego, Calif.). CCL2 enzyme-linked immunosorbent assay (ELISA) kits were obtained from R&D Systems (Minneapolis, Minn.). Lipoteichoic acid was obtained from InvivoGen (San Diego, Calif.). MMP is a synthetic bacterial lipoprotein and TLR2 ligand. Deuterated solvents (CCl₃D, D₃COD, and D₃COH) and [1-¹³C]sodium acetate were obtained from Cambridge Isotope Laboratories (Andover, Mass.). Nuclear magnetic resonance (NMR) tubes were obtained from Norrell, Landiville, N.J. Gas chromatography-mass spectrometry (GC-MS) derivatizing agents were obtained from Pierce (Rockford, Ill.).

Bacterial growth: Bacteria were grown in broth culture. P. gingivalis (ATCC 33277, type strain) was inoculated into basal medium (peptone, Trypticase, and yeast extract) supplemented with hemin and menadione (Sigma, St. Louis, Mo.) and brain heart infusion (BHI) broth. Culture purity was verified by lack of growth in aerobic culture and formation of uniform colonies when inoculated on brain heart infusion agar plates and grown under anaerobic conditions. The suspension cultures were incubated for 4 days in an anaerobic chamber flushed with N₂ (80%), CO₂ (10%), and H₂ (10%) at 37° C., and the bacteria were harvested by centrifugation (3,000×g for 20 min)

Lipid extraction, fractionation, and characterization: Lipids were extracted from lyophilized bacterial pellets. Generally, 2 to 4 grams of bacterial pellet was extracted for each semipreparative fractionation. The bacterial samples were weighed and dissolved in chloroform-methanol-water (1.33:2.67:1 [vol/vol/vol]; 2 grams of bacterial pellet in a total of 16 mL of solvent). The mixture was vortexed at 15-min intervals for 2 hours, and the mixture was supplemented with 6 mL of chloroform and 6 mL of a combination of 2 N KCl and 0.5 N K₂HPO₄. The mixture was vortexed and centrifuged at 20° C. for 45 minutes. The lower organic phase was removed and dried under nitrogen. The dried extract was reconstituted in high-performance liquid chromatography (HPLC) solvent (hexane-isopropanol-water [6:8:0.75, vol/vol/vol; 18-ml total volume) and vortexed. The sample was centrifuged at 2,500×g for 10 min, and the supernatant was removed for HPLC analysis. Semipreparative HPLC fractionation was accomplished by using a Shimadzu HPLC system equipped with dual pumps (LC-10ADvp), an automated controller (SCL-10Avp), and an in-line UV detector (SPD-10Avp). Lipids were fractionated by using normal phase separation (AscentisSi; 25 cm by 10 mm by 5 μm; Supelco Analytical) with a solvent flow of 1.8 ml/min and 1-min fractions. The effluent was monitored at 205 nm. Replicate fractionations were pooled and dried under nitrogen. The dried samples were reconstituted in HPLC solvent for MS analysis as described below. Based on the MS profiles, selected fractions were weighed and aliquoted for biological testing as described below.

HPLC fractionations also included analytical normal-phase HPLC using an AscentisSi column (25 cm by 4.6 mm by 5 mm; Supelco Analytical. This column was used with a flow of 0.5 mL/min, and effluent was monitored as described above. Lipid samples to be analyzed by NMR were first repurified by this method before dissolving them in deuterated NMR solvent.

Mass spectrometry: HPLC fractions derived either from semipreparative purification or analytical column enrichment were infused at a low flow rate (0.1 mL/min) into an ABSciex 4000 Qtrap instrument. Lipid samples were dissolved in the HPLC solvent described above. For mass spectrometry analyses, a short normal-phase column (AscentisSi; 3 cm by 2.1 mm by 5 μm; Supelco Analytical) was used for separation of the injected lipids fractions. HPLC solvent was delivered under isocratic conditions with a Shimadzu LC-10ADvp pump. Total ion chromatograms were acquired using a mass range of 100 to 1,800 atomic mass units (amu), and tandem MS (MS/MS) acquisitions used parameters optimized for the specific lipid products under analysis. Collision energies for negative ion products were typically between −30 and −55 V, depending on the precursor ion under investigation.

Fatty acid analysis of P. gingivalis lipids included transesterification or base-catalyzed hydrolysis, using either sodium methoxide (0.5 ml of 0.5 N in dry methanol; 40° C. for 20 min) or potassium hydroxide (0.5 ml of 4 N, 100° C. for 2 h), respectively. Fatty acid methyl esters were recovered by extraction into hexane (three times; 1 mL) after the addition of 1.0 mL of water to the sodium methoxide hydrolysis solution. The hexane extracts were then dried, reconstituted with N,O-bis(trimethylsilyl)trifluoroacetamide, and allowed to stand overnight before analysis. The potassium hydroxide hydrolysis reaction was stopped with the addition of 0.15 ml of concentrated HCl and 1 mL of water. The hydrolysate was extracted in triplicate with chloroform, and the combined extracts were dried under nitrogen. The dry extract was then treated to form pentafluorobenzyl ester, trimethylsilyl (TMS) ether derivatives.

Serine was hydrolyzed from the target lipids by adding 0.1 mL of 6 N HCl and heating the sample for 4 minutes in a microwave oven. The residue was dried and prepared to form methyl ester-pentafluoropropyl ether/amide derivatives for analysis according to the method of Fuchs et al. The dried samples were first treated with acetyl chloride-methanol (1:4 [vol/vol]; 100 μL; 70° C. for 45 minutes) and dried. The samples were then treated with chloroform-pentofluoropropionic anhydride (4:1 [vol/vol]; 500 μL; 100° C. for 20 min) and dried. The residues were dissolved in chloroform and analyzed by GC-MS.

Fatty acid and serine analyses: Experiments were performed on an Agilent 5975C GC-MS apparatus. Fatty acid samples were run on an Agilent HP-5 M column with a helium flow rate of 1 mL/min for both positive and negative ionization conditions. The column was generally heated from 100 to 290° C., and the injection block and transfer line were maintained at 280° and 290° C., respectively. Methyl ester-TMS products were run in the electron impact mode, and pentafluorobenzyl-TMS derivatives were run in the negative chemical ionization mode. Fatty acid quantification was accomplished by electronic integration of selected ion chromatograms. Serine quantification was accomplished using a chiral column (CP-Chirasil-1-Val; 25-m by 0.25-mm by 0.25-μm column; Agilent). The serine derivatives (pentafluoropropyl ether/amide-methyl ester derivatives) of each sample were run from 80° C. to 150° C. with the injection block and transfer line both held at 150° C. d- and l-Serine standards were prepared in parallel to determine the epimeric form of serine recovered in the serine lipids of P. gingivalis.

NMR spectroscopy: All NMR experiments were performed on Agilent VNMRS spectrometers equipped with cryogenically cooled HCN triple resonance probes at 18.8 T (¹H and ¹³C enhanced) and 11.7 T (¹H enhanced). All NMR experiments were performed at the natural abundance of ¹³C and ¹⁵N with a lipid concentration of approximately 1.5 mM at 25° C. with a sample volume of 600 μL in a 5-mm sample tube. The lipid sample was dissolved in deuterated solvent (CD₃Cl-D₃COD, 2:1 [vol/vol]), which gave narrow line widths, and the following experimental data were collected; one-dimensional (1D)¹H, 1D, and ¹³C), 2D TOCSY, 2D DQF—COSY, 2D ¹H-¹³C HSQC, 2D ¹H—¹³C HMBC, and 2D ¹H—¹³C H2BC. For evaluation of proton substitution of nitrogen, the lipid sample was dissolved in CD₃Cl-D₃COH (2:1 [vol/vol]) and analyzed as a 2D ¹H—¹⁵N HSQC spectrum and two 1D ¹H—¹⁵N HSQC spectra (¹H detected) run in a mode that only observed primary or secondary amines, respectively. The ¹H—¹³C HMBC spectrum was collected as four different experiments, each enhanced for a different ¹H—¹³C multiple-bond coupling (3 Hz, 5 Hz, 8 Hz, and 10 Hz) and added together after processing the individual spectra. The 1D ¹³C spectrum was collected at 18.8 T by using a spin-echo sequence, which gave perfectly flat baselines, along with chirp pulses to obtain uniform excitation over a 52,000-Hz sweep width (pulse sequence provided by Agilent). All NMR data were processed and analyzed using either MestReNova or NMRPipe software. The structure was reconciled through correlations in the HMBC, H2BC, DQF COSY, and TOCSY spectra along with ¹H-¹H splittings, ¹H integrations, and ¹³C chemical shifts.

Mice: Female C57BL/6 (WT) mice were purchased from Jackson Laboratory (Bar Harbor, Me.). TLR2^{-/-} mice, bred onto a C57BL/6 background, were obtained. All mice were between 6 and 12 weeks old when used.

Cell lines and assays: Human embryonic kidney cells (HEK293 cells), either nontransfected or transfected with human TLR2 or human TLR4 and stably expressing MD-2 and CD14, were purchased from InvivoGen. Cells were cultured in Dulbecco's modified Eagle's medium (DMEM; Gibco) containing 4.5 g/liter 1-glucose and 10% fetal bovine serum (FBS). The activities of specific TLR agonists were measured through a colorimetric assay for the secretory embryonic alkaline phosphatase (SEAP), a reporter gene that is linked to NF-κB activation. Measurement of SEAP activity using the Quanti-blue substrate (InvivoGen) after TLR agonist treatment was carried out in test medium (DMEM, 10% FBS) without antibiotics. NF-κB activation was expressed as a response ratio for each stimulus relative to SEAP activity in unstimulated (vehicle control) cells. For in vitro testing, all lipid preparations tested were solubilized in a 50% mixture of dimethyl sulfoxide (DMSO)-water (approximately 1.11% DMSO in the final culture medium).

In vivo assays: Wild-type (WT) C57BL/6 mice or TLR2^{-/-} mice were injected intraperitoneally (i.p.) or intravenously (i.v.) with vehicle control or specific lipids. Four hours later, blood samples were obtained from mice, and the mice were euthanized. Serum was separated from the blood samples and frozen at −80° C. until analysis. Serum samples were analyzed for levels of CCL2 by ELISA (R&D, Minneapolis, Minn.).

Assessment of lipid 654 contamination of P. gingivalis LPS: Crude LPS of P. gingivalis was prepared using the TRI reagent method of Yi and Hackett, and the crude LPS was precipitated with cold magnesium chloride in 95% ethanol. After three additional precipitations with 95% ethanol followed by precipitation with 100% ethanol, the LPS preparation was dried. Aliquots of LPS (20 μg) were dispensed into glass vials to which known amounts of serine lipid internal standard was added. The serine lipid internal standard was prepared by culturing P. gingivalis in BHI broth (described above) supplemented with 1-¹³C-labeled sodium acetate (0.5 g/liter). Bacteria were harvested by centrifugation, and total lipids were extracted and fractionated as described above. A lipid fraction with the retention time of lipid 654 was shown by electrospray ionization (ESI)-MS to have a peak mass of m/z 660. The background m/z 654 lipid in this internal standard lipid preparation was only 1.5% of the abundance of the m/z 660 species. This lipid fraction was used as an internal standard for quantifying lipid 654 contamination of P. gingivalis LPS.

The LPS:internal standard mixtures (prepared in a 0.5-ml volume in water) were supplemented with 2 mL of chloroform-methanol (1:2 [vol/vol]) and vortexed repeatedly over 1 hour. The samples were supplemented with chloroform (0.75 mL) and 0.75 mL of 2 N KCl plus 0.5 N K₂HPO₄. After vortexing, the lower chloroform phase was removed and dried. These samples were subjected to multiple reaction monitoring (MRM)-MS with instrument parameters optimized for the m/z 654-to-m/z 381 transition (lipid 654 product) and the m/z 660-to-m/z 385 transition (internal standard). The ratio of the electronically integrated peaks for these two transitions was then used to determine the amount of lipid 654 present in 20 μg of P. gingivalis LP S.

Statistical analysis: Data are expressed as the means±standard errors. Statistical testing included an analysis of variance (ANOVA) with pairwise comparisons using the Fisher least significant difference (LSD) test or the Student t test for simple group mean comparisons.

Activation of Human TLR2 by P. gingivalis Lipids

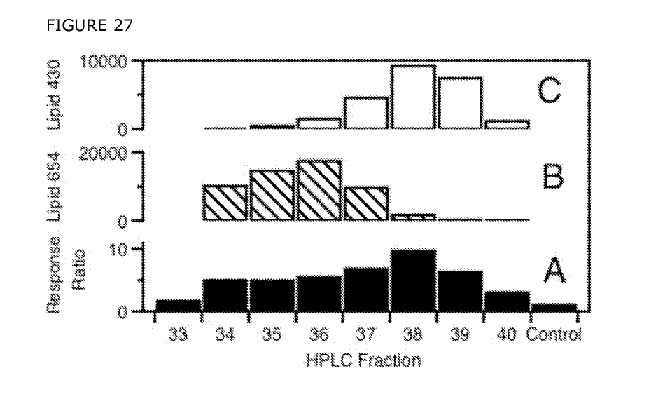

To determine the ability of P. gingivalis lipids to activate human cells via TLRs, the total lipids of P. gingivalis were fractionated by HPLC, and an aliquot of each fraction was dried and dissolved in 50% DMSO in water. Each HPLC fraction was then tested for cell activation by using HEK293 cells stably transfected with human TLR2, CD14, MD-2, and SEAP genes. In addition, the HEK293 cells naturally express variable levels of TLRs 1, 3, 5, 6, 7, and 9. The SEAP reporter gene is under the control of the beta interferon minimal promoter fused to five NF-κB and AP-1 binding sites. Stimulation with a TLR2 ligand activates NF-κB and AP-1, which induces the production of SEAP. SEAP is then quantitated via a colorimetric change in medium samples following the addition of a suitable enzyme substrate. Using this cell screen for human TLR2 engagement, each HPLC lipid fraction was screened for cell activation. HEK cell activation was observed, depicted as the response ratio relative to the vehicle control culture, only in HPLC fractions 33 through 40, as shown in FIG. 27A. Using mass spectrometry, the HPLC fractions were examined for lipid ions that correlated with the observed HEK cell activation. HEK-TLR2 cell activation directly correlated with levels of lipids that produced negative ions of m/z 654, 640, and 626 (here termed lipid 654) or negative ions of m/z 430, 416, and 402 (here termed lipid 430). The ion abundances of the m/z 654 and 430 negative ions are depicted for HPLC fractions 33 through 40 in FIG. 27B and FIG. 27C, and these negative ions represent the most abundant lipid species within lipid 654 and lipid 430 classes. Although these TLR2-activating HPLC fractions contained small amounts of previously characterized phosphatidylethanolamine and phosphoethanolamine dihydroceramide lipids as determined by mass spectrometric analysis, the minimal levels of these contaminating lipids did not correlate with TLR2 cell activation.

MS and NMR Analysis of Lipid 654.

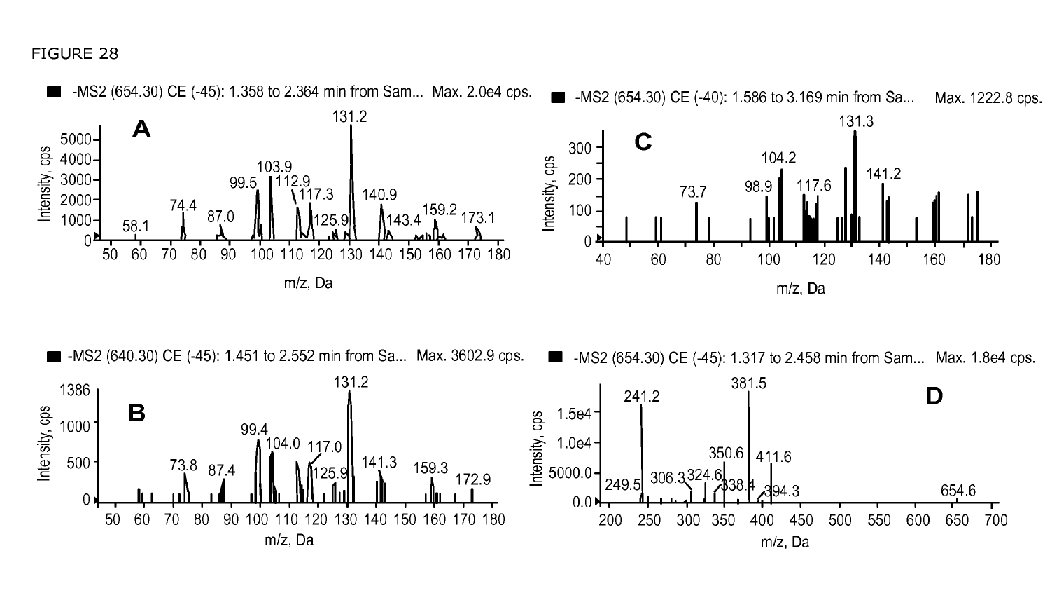

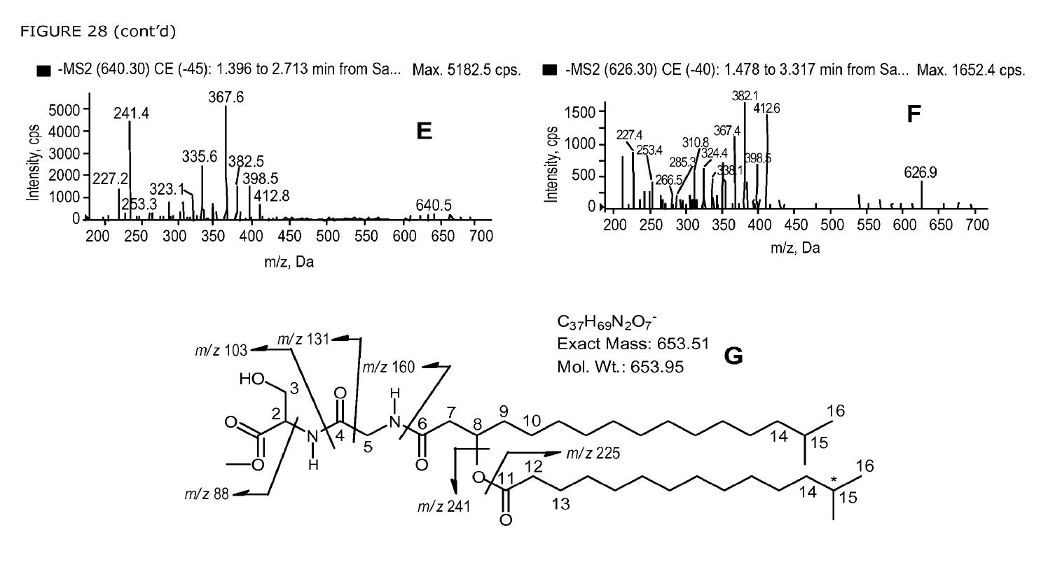

The negative ion MS/MS analysis of the m/z 654, 640, and 626 precursor ions revealed the MS/MS spectra shown in FIG. 28A-F. The lipid structure in FIG. 28G shows the most abundant species of the lipid 654 class. The structure depicts two fatty acids linked by a β-carbon ester, and the hydroxyl fatty acid is held in amide linkage to a dipeptide composed of glycine and a terminal serine. Other fatty acids can be substituted into this lipid class as described below, and these alternate fatty acid substitutions account for the m/z 654, 640, and 626 parent molecules. Lipid extracts from the broth medium used to culture P. gingivalis showed no m/z 654, 640, or 626 ions. Positive ion mass spectra revealed molecular ion masses of m/z 656, 643, and 628, indicating at least one elemental nitrogen atom in the component molecular species of the lipid 654 class.

The multiple-bond ¹H-¹³C correlations from the HMBC, ¹H-¹H correlations from the DQF-COSY and TOCSY experiments, ¹³C chemical shifts, ¹H integrations, and ¹H-¹H coupling constants were sufficient to map the structure of the lipid for all atoms except the central CH₂ groups in the fatty acid aliphatic chains, due to significant overlap in the NMR spectra. Information used in the structure determination is shown in Table 3, with carbon numbers corresponding to those listed in the chemical structure in FIG. 28G. Integration of the large overlapped peak in the ¹H 1D NMR spectrum corresponding to 17 CH₂ groups yielded a value of 35.9, slightly higher than the expected 34 but well within the expected error and consistent with the length of fatty acid aliphatic chains. Coupling patterns and integrations confirmed that approximately 85% of the fatty acids are isobranched, with 15% being anteisobranched. The ¹H—¹⁵N HSQC confirmed that there were two protonated nitrogens, and both were shown to be secondary amines. The 1D ¹³C NMR spectrum confirmed the presence of four carbonyl carbons, although the signal for carbonyl 1 was weak due to a longer T₁ relaxation time (a 3-second recycle delay was used). The four carbonyls were also observed by long-range couplings in the ¹H—¹³C HMBC. The three methylene groups at atoms C-3, C-5, and C-7 gave unique chemical shifts for the two protons, demonstrating a lack of bond rotation. From these proton and carbon assignments, listed in Table 3, together with the mass spectrometric results, it was confirmed that this lipid class represents the previously reported lipid called flavolipin. However, as discussed below, lipid 654 is clearly distinct from flavolipin, both in its biological activity and in the range of bacteria from which it can be derived.

MS Analysis of Lipid 430:

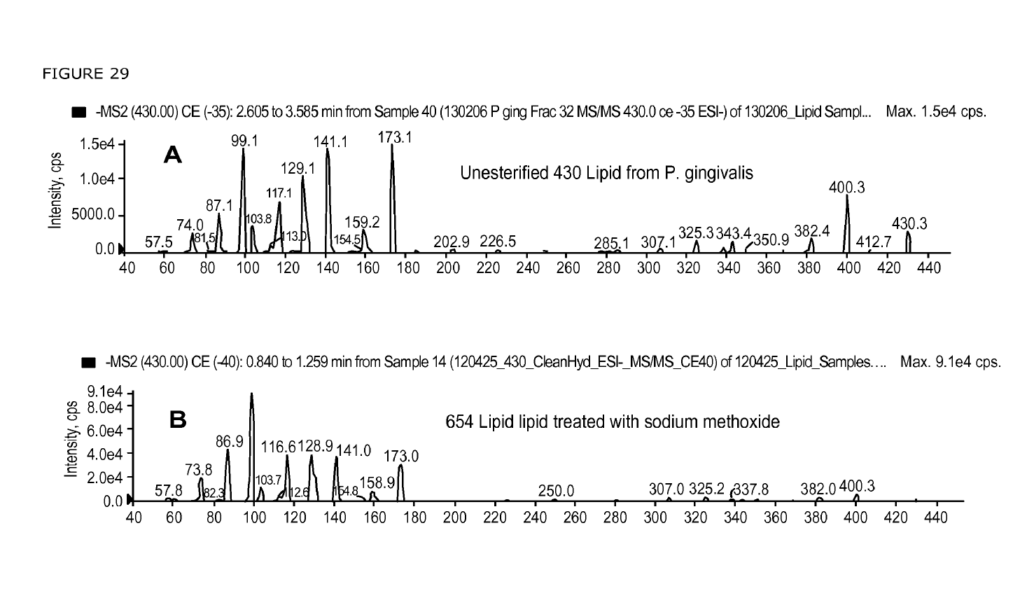

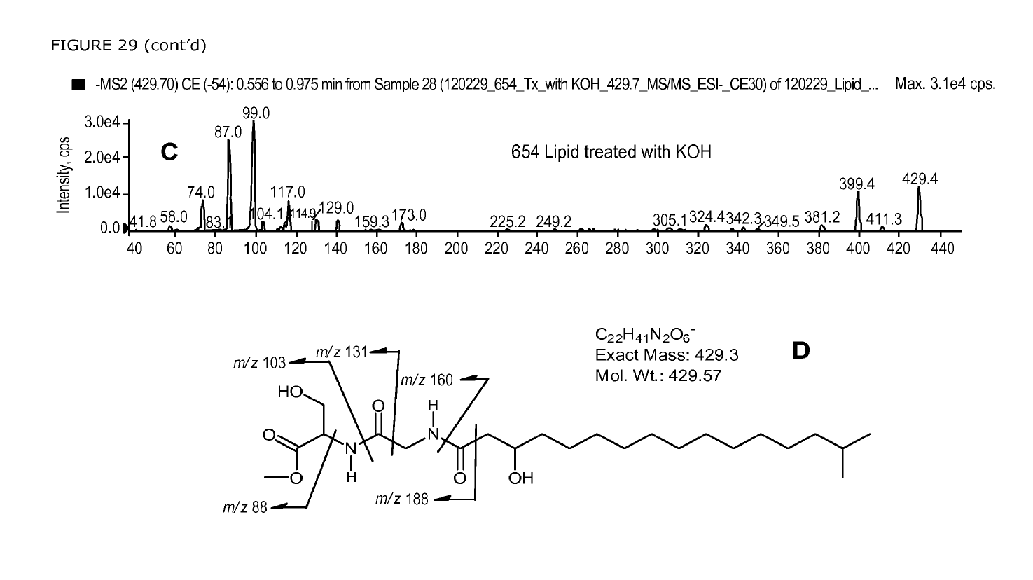

To analyze whether the molecular weights of the three major lipid species within the lipid 430 class are consistent with the loss of an esterified fatty acid from the respective constituent lipid species of the lipid 654 class, a sample of HPLC lipid fraction 35 containing highly enriched lipid 654 was subjected to base-catalyzed hydrolysis with either sodium methoxide or KOH in order to release ester-linked fatty acids. Both hydrolysis methods yielded low levels of lipids that produced negative ions of m/z 430, 416, and very small amounts of 402 as measured by ESI-MS (FIG. 29). The nonesterified 430 lipid class recovered in HPLC fraction 39 of the total lipid extract of P. gingivalis (FIG. 29A) showed an MS/MS spectrum similar to the m/z 430 lipids recovered after sodium methoxide treatment of lipid 654 (FIG. 29B) or KOH treatment of lipid 654 (FIG. 29C). The m/z 430 and 416 negative ions of the nonesterified lipid 430 were evaluated by MS/MS and revealed low-mass product ions (<200 amu), similar to those produced from m/z 654, 640, and 626 lipid species (FIG. 28). By increasing the collision energy for gas-phase fragmentation of precursor ions, the low-mass ion fragments (<200 amu) of the m/z 654 precursor increased in abundance to that shown for the lipid 430 fragmentations shown in FIG. 29. As with the lipid 654 class, lipid extracts from the broth medium used to culture P. gingivalis showed no m/z 430, 416, or 402 ions. These results demonstrate that the lipid 430 class represents the deesterified or nonesterified lipid 654 class (FIG. 29D, lipid 430 structure) and that the three lipid species contain the same amino acids within their respective head groups. However, the base-catalyzed hydrolysis of either the lipid 654 or lipid 430 classes eliminated their ability to activate TLR2-expressing HEK293 cells due to substantial breakdown of the lipid 654/430 products, as verified by thin-layer chromatography.

Fatty Acid and Serine Constituents in the Lipid 654 Class.

Hexane extraction of the sodium methoxide-treated lipid 654, followed by GC-MS analysis, yielded fatty acid methyl esters, including branched CH₃—C_{15:0} with lesser amounts of CH₃—C_{14:0} (2.3%) and CH₃-isobranched C_{13:0} (0.44%). Hexane extracts of the KOH-treated lipid 654 were processed to form pentafluorobenyl ester, TMS ether derivatives, and were prepared in parallel with synthetic standards of anteisobranched and isobranched C_{15:0} and 3-OH fatty acid standards. Negative-ion GC-MS revealed that the C_{15:0} is approximately 88% isobranched, with the remainder anteisobranched C_{15:0}. Straight-chain C_{15:0} was not observed. Negative-ion GC-MS of the 3-OH fatty acids revealed 69.8% as 3-OH iso-C_{17:0}, 25.5% as 3-OH C_{16:0}, and 4.7% as 3-OH iso-C_{15:0}. By comparison, the average distribution of lipid species within the lipid 654 class was as follows: m/z 654 (61.8%), m/z 640 (31%), and m/z 626 (7.2%) ions. Therefore, the distribution of the hydroxy fatty acids, rather than the ester-linked fatty acids, in the lipid 654 class appears to account for the distribution of its three characteristic lipid species. The epimeric configuration of serine in the 654 lipid class was determined by chiral GC-MS analysis. This analysis demonstrated that the 654 lipid class contains only 1-serine. The stereochemistry of C-8 (FIG. 28G) has not been determined for lipid 654, nor has the stereochemistry of C-14 for the anteiso-C_{15:0} fatty acid been determined

Lipid 654 and Lipid 430 Effects In Vitro: Dose Responses and Biological Activities Relative to Other Major Lipid Classes of P. gingivalis.

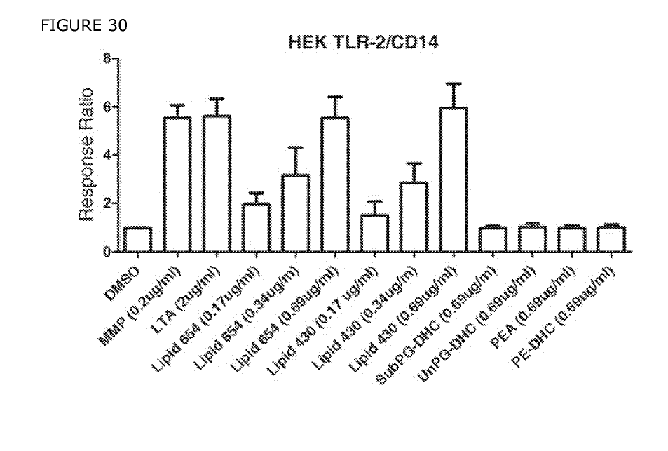

The biological activity dose-response characteristics of lipid 654 and lipid 430 were evaluated and the responses were compared with well-characterized TLR2 agonists as well as other prevalent lipid classes of P. gingivalis. The HPLC fractions containing either highly enriched lipid 654 (fraction 35) or lipid 430 (fraction 39) were evaluated for their abilities to activate TLR2-expressing HEK293 cells compared with the substituted phosphoglycerol dihydroceramides (subPG-DHC), unsubstituted phosphoglycerol dihydroceramide lipids (unPG-DHC), phosphoethanolamine dihydroceramide lipids (PE-DHC), and phosphatidylethanolamine (PEA) lipids of P. gingivalis (FIG. 30). Compared with the known TLR2 ligand positive controls MMP and lipoteichoic acid (LTA), lipid 654 and lipid 430 promoted significant HEK cell activation over the control cells (DMSO-treated cells). MMP (molecular weight of 1,269.82) was used at a concentration of 0.2 μg/mL, or 0.158 μM. Using the molecular weights and distributions of the three lipid species within each lipid class, lipid 654 and lipid 430 at a concentration of 0.69 μg/mL represented doses of 1.066 μM and 1.621 μM, respectively. Lipid 654 and lipid 430 used at a concentration of 0.17 μg/ml represented 0.259 μM and 0.395 μM, respectively. The molecular weight of LTA was not provided by the supplier, and the molar concentration could not be calculated. All other major lipid classes of P. gingivalis, previously isolated to very high purity, showed little capacity to activate TLR2 in HEK cells. Therefore, the phosphorylated dihydroceramide lipids of P. gingivalis do not account for the HEK cell activation observed in the total lipid extract of P. gingivalis. Instead, the HPLC fractions containing lipid 654 and lipid 430 accounted for the majority of the HEK cell activation observed with the total lipid extract. FIG. 30 also shows the dose-response characteristics of lipid 654 and lipid 430 classes and confirms that these lipid classes are capable of activating HEK cells at low concentrations.

Lipid 654 and Lipid 430 In Vitro: TLR2 Dependence of Biological Activities.

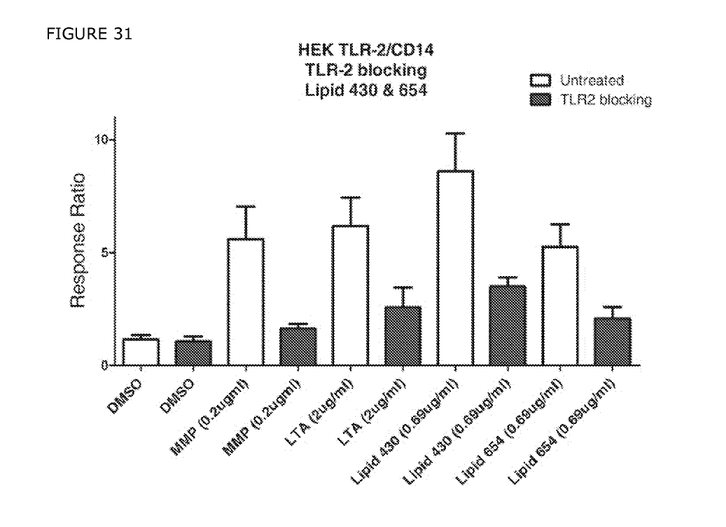

As shown in FIG. 31, MMP and LTA demonstrated TLR2 cell activation that was inhibited by pretreatment with anti-human TLR2 antibody. HEK-TLR2 cell responses to lipid 654 or lipid 430 preparations of P. gingivalis were also significantly inhibited by pretreatment with anti-human TLR2 antibody (FIG. 31). These results showed that lipid 654 and lipid 430 are ligands for human TLR2.

Lack of TLR4 Activation by Lipid 654 and Lipid 430.

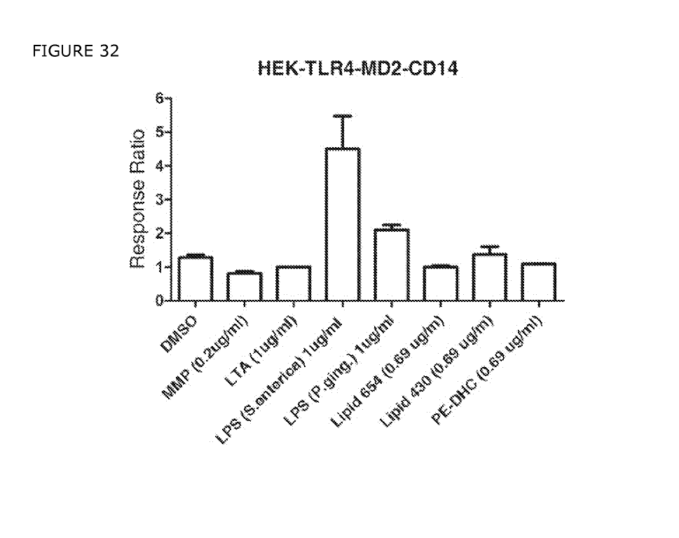

To test whether lipid 654 and lipid 430 can function as ligands for TLR4, HEK293 cells transfected with the human TLR4, CD14, and MD-2 genes but not expressing TLR2 were utilized. As shown in FIG. 32, the known TLR4 agonist LPS (derived either from Salmonella enterica or P. gingivalis) demonstrated the ability to stimulate the TLR4-expressing HEK293 cells. LPS from P. gingivalis was considerably weaker than enterobacterial LPS in stimulating HEK cells. In contrast, the TLR2 agonists MMP and LTA showed no activity. Most importantly, lipid 654 and lipid 430 showed no capacity to activate the TLR4-expressing cell line. These results indicate that lipid 654 and lipid 430, in contrast to flavolipin, can activate via TLR2 but are unable to function as TLR4 ligands. In additional studies, it was demonstrated that the HEK null cells (HEK cells with the SEAP reporter gene but without transfected TLRs) also do not respond to either the TLR4 or TLR2 agonists.

Bioactivities of Lipid 654 and Lipid 430 In Vivo.

Studies were performed to confirm the in vitro functional effects of lipid 654 and lipid 430 by using in vivo approaches. Mice were injected with lipid 654 or lipid 430 and the effect on serum levels of the chemokine CCL2 (also known as monocyte chemoattractant protein 1) was analyzed. CCL2 plays a major role in mediating the migration of inflammatory macrophages into tissue sites of inflammation, and it has been previously documented that administration of TLR agonists to mice can result in expression of serum CCL2. Furthermore, this chemokine has been suggested to be important in the pathogenesis of autoimmune diseases and has been shown to be critical for the development of experimental autoimmune encephalomyelitis (EAE), the murine model of multiple sclerosis, and is also believed to be critical in the pathogenesis of human multiple sclerosis.

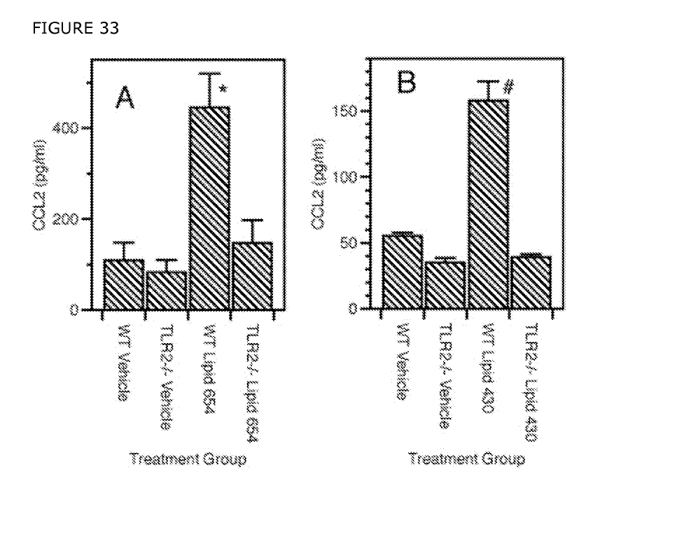

Lipid 654 was injected i.p. in 50% DMSO and lipid 430 was injected i.v. in phosphate-buffered saline (PBS). WT female C57BL/6 mice and TLR2^{-/-} female mice were injected with either vehicle, lipid 654, or lipid 430. Four hours later, serum was recovered from these mice and analyzed for levels of CCL2. As shown in FIG. 33, lipid 654 induced a significant increase in serum levels of CCL2 in WT mice but failed to do so when injected into TLR2^{-/-} mice (FIG. 33A). The same was true for lipid 430. Lipid 430 induced a significant increase in serum levels of CCL2 in WT mice but failed to do so when injected into TLR2^{-/-} mice (FIG. 33B). These results demonstrated that both lipid 654 and lipid 430 have proinflammatory effects in vivo and, further, that these effects are dependent on TLR2.

Lipid 654 Contamination of P. gingivalis LPS.

The LPS extract of P. gingivalis was shown to contain 3.82%±0.18% lipid 654 (mean±standard deviation; n=3). This LPS preparation, at a concentration of 0.69 μg/mL, produced a 1.8-fold increase in TLR2 activation in HEK cells, whereas MMP (0.2 μg/mL) produced a 3.7-fold increase. The final concentration of lipid 654 in this LPS assay preparation was calculated to be 0.039 μM, whereas the concentration of MMP used in the assay was 0.158 μM.

Different arrangements and combinations of the elements and the features described herein are possible. Similarly, some features and subcombinations are useful and may be employed without reference to other features and subcombinations. Embodiments of the invention and examples have been described for illustrative and not restrictive purposes, and alternative embodiments will become apparent to readers of this patent. Accordingly, the present invention is not limited to the embodiments described above or depicted in the drawings, and various embodiments and modifications can be made without departing from the scope of the claims below.

TABLE 1
EAE disease assessment
A.

| | EtOH | | PE DHC | | | |
|---|---|---|---|---|---|---|
| Mouse Strain | CDI | MDD | CDI | MDD | P value | n |
| Wild Type | 10.1 | 0.5 | 19.8 | 1.0 | 0.001 | 28 |
| TLR2^{-/-} | 7.6 | 0.4 | 8.3 | 0.4 | 0.306 | 15 |
| IL-15^{-/-} | 9.4 | 0.5 | 24.5 | 1.2 | 0.001 | 10 |
| IL-15Rα^{-/-} | 7.0 | 0.4 | 18.7 | 0.9 | 0.0077 | 12 |

B.

| | | | EtOH | PE DHC | | |
|---|---|---|---|---|---|---|
| | EtOH | PE DHC | Mean Maximum | | EtOH | PE DHC |
| Mouse Strain | Mean Incidence | | Severity | | Mean Day of Onset | |
| Wild Type | 58.6 | 75 | 3.2 | 3.6 | 14.4 | 12.1 |
| TLR2^{-/-} | 42.1 | 46.6 | 3.0 | 2.9 | 14.4 | 14.7 |
| IL-15^{-/-} | 60 | 90 | 3.1 | 3.7 | 14.7 | 13.0 |
| IL-15Rα^{-/-} | 66 | 100 | 2.7 | 2.9 | 15.1 | 13.8 |

A. The cumulative disease index (CDI) was obtained by summing the daily average disease scores of each experimental group through Day 20. A mean of these daily disease scores (Mean Daily Disease - MDD) was calculated based on the 20 days of observation. The MDD scores were compared using the Wilcoxin Signed Rank tests for two samples. n is the total number of mice studied in each experimental group.
B. Mean incidence of disease is represented as a percentage and was calculated by dividing the number of mice within each group that developed clinical signs of EAE by the total number of mice in that group. Disease incidence frequencies were compared using Chi square analysis. Mean maximum severity of EAE was calculated for mice that developed EAE by taking the highest score observed for each mouse in the 20-day observation period and averaging these values among mice in the same group. Statistical significance was determined using the Wilcoxin Signed Rank test. Mean day of onset of EAE was calculated for mice that developed EAE by using the first day of observance of signs of EAE as the value and averaging these values among mice in the same group. Statistical significance was determined using the Student's t-test.

TABLE 2
Spinal Cord T cells
Mice were sampled from 3 different experiments and sacrificed
1-5 days after onset of signs of EAE. Mononuclear cells were derived
from the spinal cords, stained for CD4 and Foxp3, and
evaluated by flow cytometry. % CD4 represents the % CD4+
T cells within the total spinal cord mononuclear cells. % Foxp3
represents the The PE DHC fraction altered the composition of cells
infiltrating the spinal cords of mice with EAE. % Foxp3+ T
cells after gating on CD4+ T cells.

| Treatment- | DAYS AFTER | DISEASE | % CD4+ in | % Foxp3+ in |
|---|---|---|---|---|
| mouse | ONSET of EAE | GRADE | spinal cord | spinal cord |
| ETOH-1 | 2 | 1.0 | 49.60 | 8.31 |
| ETOH-2 | 4 | 2.8 | 55.23 | 5.71 |
| ETOH-3 | 5 | 3.3 | 49.40 | 5.57 |
| ETOH-4 | 2 | 2.7 | 44.88 | 7.52 |
| ETOH-5 | 1 | 1.0 | 32.98 | 6.01 |
| ETOH-6 | 1 | 2.0 | 15.18 | 7.06 |
| | | | Mean = 41.21 | Mean = 6.70 |
| | | | +/− 14.78 | +/− 1.11 |
| PE DHC-1 | 5 | 3.3 | 72.00 | 4.53 |
| PE DHC-2 | 4 | 3.5 | 38.00 | 7.52 |
| PE DHC-3 | 5 | 2.9 | 68.86 | 2.75 |
| PE DHC-4 | 4 | 3.3 | 49.54 | 3.45 |
| PE DHC-5 | 2 | 2.9 | 40.83 | 3.21 |
| PE DHC-6 | 4 | 3.0 | 46.50 | 4.27 |
| | | | Mean = 52.62 | Mean = 4.29 |
| | | | +/− 14.42 | +/− 1.72 |
| | | | p = 0.1635 | p = 0.0397 |

TABLE 3
Structure of Lipid 654 (Determined by multiple NMR spectral analyses)

| | | | | Hetero- | | | | |
|---|---|---|---|---|---|---|---|---|
| | | | Proton | Atom | 1H-1H | | | |
| | | | Shift | Shift | Splittings | DQF-COSY | TOCSY | HMBC |
| | Group | Integration | (ppm) | (ppm) | (Hz) | Correlations | Correlations | Correlations |
| 1 | C═O | | | 174.877 | | | | 2, Ser N ⁱ |
| 2 | CH | 0.94 | 4.465 | 57.571 | 3.76 (3, 3′) | 3, 3′, 17 | 3, 3′, 17 | 1, 3, 4 |
| 3 | CH₂ ^{b} | 2.16^{c} | 3.873, | 64.582 | 11.58 (geminal), | 2, 3, 3′ | 2, 3, 3′, 17 | 2, ⁱ |
| | | | 3.794 | | [3, 47, 4.19] | | | |
| 4 | C═O | | | 172.033 | | | | 2, Ser N, 5 |
| 5 | CH₂ ^{b} | 1.77^{c} | 3.843, | 45.209 | 16.76 (geminal^{j}) | 5, 5′ | 5, 5′ | 4, 6 |
| | | | 3.801 | | | | | |
| 6 | C═O | | | 174.142 | | | | 5, Gly N, 7, |
| | | | | | | | | 8 |
| 7 | CH₂ ^{b} | 1.97 | 2.460, | 43.506 | 14.53 (geminal^{j}), | 8 | 8, 9, 10, FA | 6, 8, 9 |
| | | | 2.412 | | [7.58, 5.07] | | | |
| 8 | CH | 1.0 | 5.122 | 73.82 | ^{g} | 7, 7′, 9 | 7, 7′, 9, 10, | 6, 7, 9, 10 |
| | | | | | | | FA | |
| 9 | CH₂ | 4.28 ^{d,e} | 1.533 | 36.711 | ^{g} | 8, 10 | 7, 7′, 8, 10, | 7, 8, 10, FA |
| | | | | | | | FA | |
| 10 | CH₂ | ^{e} | 1.225 | 27.668 | ^{g} | 9, FA^{h} | 7, 7′, 8, 9, | 9, FA |
| | | | | | | | FA | |
| 11 | C═O | | | 176.732 | | | | 8, 12, 13 |
| 12 | CH₂ | 2.00 | 2.222 | 36.984 | 7.5, 2.97, and | 13 | 13, FA | 11, 13, FA |
| | | | | | 2.66 | | | |
| 13 | CH₂ | 4.28 ^{d,e} | 1.509 | 27.508 | ^{g} | 12, FA | 12, FA | 11, 12, FA |
| 14 | (CH₂)₂ | 3.91 | 1.051 | 41.545 | ^{g} | FA, 15 | FA, 15, 16 | FA, 15, 16 |
| 15 | (CH)₂ | 1.69^{e} | 1.430 | 30.437 | 6.68 | 14, 16 | FA, 14, 16 | FA, 14, 16 |
| 16 | (CH₃)₄ | 11.80 | 0.778 | 29.947 | 6.68 | 15 | FA, 14, 15 | 14, 15 |
| Ser | NH-Ser | 1.0 | 7.504 | 112.19 | | 2 | 2, 3, 3′ | 2, 1, 4 |
| Gly | NH-Gly | 1.0 | 7.756 | 110.82 | | 5, 5′ | 5, 5′ | 5, 6 |

NMR Legend:
^{a} For NMR evaluation of lipid 654, a highly purified lipid sample was processed as described in Materials and Methods. For the NMR proton assignments, all integrations were normalized to the proton on C-8 (integrated to 1.0). The carbon assignments shown in FIG. 28G correspond to the carbon numbers listed in the first column.
^{b} Methylene groups for C-3, C-5, and C-7 gave unique proton chemical shifts, indicating a lack of proton rotation.
^{c}Proton resonances for C-3 and C-5 overlap, causing the individual integrations to be slightly deviated, but taken together they integrated to 4.04, a result very close to the predicted value of 4.0.
^{d} Proton resonances for C-9 and C-13 overlap and were integrated together.
^{e}The integration of the peak for C-15 yielded 1.69, 15% lower than the predicted 2.0, indicating that approximately 85% of the fatty acids are isobranched. Integration of peaks C-9 and C-13 yielded 4.28, which is slightly higher than the predicted 4.0 due to an extra signal from the approximately 15% of anteisobranched fatty acid.
^{f} Peak overlaps, with the intense peak from the fatty acid. The total integration of the intense peak was 35.92, slightly higher than the predicted 34.
^{g} Couplings could not be determined due to overlap.
^{h}FA, fatty acid.
ⁱ Coupling between C-1 and C-3 was not observed due to overlap between the proton chemical shifts of C-3 and C-5.
^{j}gem, geminal protons.

1-76. (canceled)

77. A method for detecting an autoimmune condition in a test subject, comprising:
extracting lipids from a test sample obtained from the test subject, thereby producing extracted lipids;
analyzing the extracted lipids by mass spectrometry to quantify levels of one or more subclasses of bacterial L-serine containing lipids in the test sample; and,
comparing the quantified levels of the one or more subclasses of bacterial L-serine containing lipids in the test sample to levels of the one or more subclasses of bacterial L-serine containing lipids in a control sample;
wherein altered levels of the one or more subclasses of bacterial L-serine containing lipids in the test sample, in comparison to the levels in the control sample is indicative of a presence of the autoimmune condition in the test subject,
wherein the test sample and the control samples are corresponding samples of human or animal bodily fluids or tissues,
and wherein the control sample is obtained from a control human or animal subject not having the autoimmune condition or having low activity of the autoimmune condition.

78. The method of claim 77, wherein the one or more subclasses of bacterial L-serine containing lipids include one or both of Lipid 654 and Lipid 430.

79. The method of claim 78, wherein the altered levels is a decreased level of one or both of Lipid 654 and Lipid 430 in the test sample, in comparison to the levels in the control sample, wherein the decreased level is indicative of the presence of the autoimmune condition in the test subject, and wherein the autoimmune condition is multiple sclerosis.

80. The method of claim 77, wherein the test sample is a tooth sample, a sample of brain tissue, kidney tissue, synovial tissue, skin, blood, serum, plasma, cerebrospinal fluid (CSF) or synovial fluid.

81. The method of claim 77, wherein the autoimmune condition is multiple sclerosis, rheumatoid arthritis, systemic lupus erythematosus or type 1 diabetes.

82. The method of claim 77, wherein the step of analyzing the extracted lipids by mass spectroscopy comprises:
chromatographically separating the extracted lipids;
ionizing the chromatographically separated lipids to produce lipid ions;
detecting the lipid ions by mass spectrometry; and,
quantifying the levels of the one or more subclasses of bacterial L-serine containing lipids from the detected lipid ions by comparison to a serine lipid internal standard obtained from cultured bacteria.

83. The method of claim 77, wherein the levels of the one or more subclasses of bacterial L-serine containing lipids in the test sample and in the control sample are quantified during the same mass spectroscopy run.

84. A method of modulating an autoimmune condition in a subject, comprising administering to the subject a composition comprising one or both of Lipid 654 and Lipid 430 in an amount effective to modulate the autoimmune condition in the subject, wherein the subject is a human or an animal.

85. The method of claim 84, wherein the autoimmune condition is multiple sclerosis, rheumatoid arthritis, systemic lupus erythematosus or type 1 diabetes.

86. The method of claim 84, wherein the subject is the test subject, and, wherein prior to the step of administering, the autoimmune condition in the test subject was detected by the method comprising:
extracting lipids from a test sample obtained from the test subject, thereby producing extracted lipids;
analyzing the extracted lipids by mass spectrometry to quantify levels of one or more subclasses of bacterial L-serine containing lipids in the test sample; and,
comparing the quantified levels of the one or more subclasses of bacterial L-serine containing lipids in the test sample to levels of the one or more subclasses of bacterial L-serine containing lipids in a control sample;
wherein altered levels of the one or more subclasses of bacterial L-serine containing lipids in the test sample, in comparison to the levels in the control sample is indicative of a presence of the autoimmune condition in the test subject,
wherein the test sample and the control samples are corresponding samples of human or animal bodily fluids or tissues,
and wherein the control sample is obtained from a control human or animal subject not having the autoimmune condition or having low activity of the autoimmune condition.

87. An antibody capable of specifically binding to a subclass of bacterial L-serine containing lipids.

88. The antibody of claim 87, wherein the subclass of bacterial L-serine containing lipids is Lipid 654 or Lipid 430.

89. A method for detecting an autoimmune condition in a test subject, comprising:
performing an immunoassay to quantify levels of a subclass of bacterial L-serine containing lipids in a test sample obtained from the test subject, comprising contacting the sample with the antibody of claim 87; and,
comparing the quantified levels of the subclass of bacterial L-serine containing lipids in the test sample to levels of the subclass of bacterial L-serine containing lipids in a control sample;
wherein altered levels of the subclass of bacterial L-serine containing lipids in the test sample, in comparison to the levels in the control sample is indicative of a presence of the autoimmune condition in the test subject,
wherein the test sample and the control sample are corresponding samples of human or animal bodily fluids or tissues,
and wherein the control sample is obtained from a control human or animal subject not having the autoimmune condition or having low activity of the autoimmune condition.

90. The method of claim 89, wherein the subclass of bacterial L-serine containing lipids is Lipid 654 or Lipid 430.

91. The method of claim 89, wherein the altered levels is a decreased level of one or both of Lipid 654 and Lipid 430 in the test sample, in comparison to the levels in the control sample, wherein the decreased level is indicative of the presence of the autoimmune condition in the test subject, and wherein the autoimmune condition is multiple sclerosis.

92. The method of claim 89, wherein the test sample is a tooth sample, a sample of brain tissue, kidney tissue, synovial tissue, skin, blood, serum, plasma, cerebrospinal fluid (CSF) or synovial fluid.

93. The method of claim 89 wherein the autoimmune condition is multiple sclerosis, rheumatoid arthritis, systemic lupus erythematosus or type 1 diabetes.

94. The method of claim 89, wherein the immunoassay is ELISA.

95. A method of modulating an autoimmune condition in a subject, comprising administering to the subject a composition comprising the antibody of claim 88 in an amount effective to modulate the autoimmune condition in the subject, wherein the subject is a human or an animal.

96. An immunoassay kit comprising the antibody of claim 87.
